(19) United States
(12) Patent Application Publication
(10) Pub. No.: US 2026/0011097 A1
(43) Pub. Date: Jan. 8, 2026

(54) USE OF THREE-DIMENSIONAL MODELS OF SPECIMENS AND DEFECTS TO ENHANCE COMMUNICATION AND DOCUMENTATION OF FROZEN AND PERMANENT SECTIONS IN CANCER SURGERY

(71) Applicant: ICAHN SCHOOL OF MEDICINE AT MOUNT SINAI, New York, NY (US)

(72) Inventors: Mark Urken, New York, NY (US); Margaret Brandwein, New York, NY (US); Michael Karasick, New York, NY (US); Kayvon Sharif, New York, NY (US)

(21) Appl. No.: 18/873,287

(22) PCT Filed: Jun. 9, 2023
(86) PCT No.: PCT/US2023/068201

§ 371 Date: Dec. 9, 2024

Related U.S. Application Data
(60) Provisional application No. 63/351,292, filed on Jun. 10, 2022.

(51) Int. Cl. G06T 19/20; G06F 3/04815; G06F 3/04845; G06T 7/73; G16H 30/40

(52) U.S. Cl. CPC G06T 19/20; G06F 3/04815; G06F 3/04845; G06T 7/75; G16H 30/40; G06T 2200/24; G06T 2207/30024; G06T 2207/30096; G06T 2210/41; G06T 2219/004; G06T 2219/2004; G06T 2219/2016

(57) ABSTRACT

Systems and methods for localizing close or positive margins in a tumor specimen from a subject are provided. A three-dimensional representation of the specimen is obtained, where the representation comprises more than 1000 three-dimensional points that define a boundary of the specimen. A plurality of annotations to the representation is received, including a first subset of annotations indicating an orientation of the representation relative to the subject, a second subset of annotations indicating a plurality of tissue sections from the specimen, and a third subset of annotations providing a margin status for respective tissue sections in the plurality of tissue sections, where the margin status indicates close or positive margins based upon pathological analysis of the respective tissue sections. A composite three-dimensional representation of the tumor specimen is formed by modifying the first three-dimensional representation of the tumor specimen to include the plurality of annotations.

### CROSS REFERENCE TO RELATED APPLICATIONS

This application claims priority to U.S. Provisional Patent Application Ser. No. 63/351,292, filed Jun. 10, 2022, which is hereby incorporated by reference in its entirety.

### TECHNICAL FIELD

The disclosure relates generally to visualizing three-dimensional representations of tumor specimens and surgical defects to enhance communication and documentation of pathologic reporting, in particular a method of bidirectional communication between the operating room and the frozen section pathology laboratory.

### BACKGROUND

Adequate resection margins are cornerstone to the successful treatment of most head and neck cancers; the status of resection margins dictates postoperative treatment decisions. Intraoperative resection specimen hand-off from surgeon to pathologist requires mutual understanding of the anatomic landmarks and precise communication of the margin mapping results. Once the specimen is dissected and margins of concern are identified, the pathologist must communicate, accurately and efficiently, which regions require further surgery. Currently, surgeons need to scrub out, leave the operating room (OR) to review the actual dissected specimen in the frozen section lab, correlate margin results, then scrub again and return to the OR, thus adding at least 20 to 40 minutes of anesthesia time to the procedure. The above process may need to be repeated again if positive margins are identified and supplemental margins have to be taken and reviewed. In addition, the pathologist is usually unaware of the details of the surgical defect from which the specimen was resected. The precise location and breadth of supplemental margins has not been documented other than for the surgeon to state that a supplemental piece of tissue was taken to “clear” a close or positive margin identified on the specimen. The current standard of intraoperative margin mapping, pathologist/surgeon communication, and process documentation has not seen any major advancements since the early 20th century.

### SUMMARY

Given the above background, the present disclosure provides systems and methods for a disruptive model that enhances surgeon/pathologist communication, reduces the need for surgeons to leave the OR, and documents location of harvested supplemental margins. In some embodiments, this model provides transparency with reconciliation of margins of concern, and decreases potential for communication errors. In some implementations, the disclosed systems and methods are used to produce a report that can facilitate treatment planning for radiation oncologists. This mode of communication is especially important when discussing large, complex multi-planar resections, where words and two-dimensional diagrams can fall short. In some implementations, the present disclosure provides an interactive visual communication tool between surgeon and pathologist which enables various processes.

For instance, in an example embodiment, resection specimens are scanned with a 3-D camera in the frozen section (FS) lab prior to pathologist dissection, producing a 3-D specimen representation which can be rotated in space. Various software can be used for anatomic labeling and annotation of the 3-D representation. After specimen dissection by the pathologist, slides are examined, margin data is generated, and the results are added to the representation. These results are reported to the surgeon by videoconferencing between the OR and FS lab.

In another example embodiment, the patient's surgical defect is also scanned in the OR with a 3-D camera and a map of the surgical defect is produced. In some implementations, software can be used which allows for anatomic labeling of the defect and annotation of supplemental margins if needed. In some implementations, a dynamic relational anatomical model incorporating surgical defect and resection specimen can be developed using nonrigid registration between the two coordinate systems. The pathologist can point to a region on the specimen representation and the corresponding region in the defect scan is highlighted. Additionally, in some implementations, virtual representations of the supplemental margins are generated by the software and manipulated and “snapped” onto the specimen representation as well as the defect. This will be akin to fitting together puzzle pieces. This produces a representation of the final extent of surgery.

Advantageously, the systems and methods disclosed herein may demonstrate proof of concept of this new intraoperative margin mapping process and illustrate the utility of software used to support the process. Possible benefits include decreased anesthesia time due to enhanced surgeon/pathologist communication. Additionally, the systems and methods disclosed herein are likely to be highly adaptable to existing surgical methods; in particular, the generation of 3D representations does not change the manner in which surgeons determine the extent of the initial resection, or how pathologists detect cancer. Therefore, this communication tool can be readily deployed once developed.

Accordingly, one aspect of the present disclosure provides a system comprising, at a first electronic device, a display, an input device, one or more processors, memory, and one or more programs, where the one or more programs are stored in the memory and configured to be executed by the one or more processors, the one or more programs including instructions for a method for localizing close or positive margins in a tumor specimen from a subject. In some embodiments, the method comprises obtaining a first three-dimensional representation of the tumor specimen and a first plurality of annotations to the first three-dimensional representation of the tumor specimen; receiving, from a second electronic device, a first three-dimensional representation of a tumor resection defect in the subject that corresponds to the tumor specimen; displaying, on the display, a customizable user interface comprising, for each respective tissue section in a corresponding plurality of tissue sections from the tumor specimen, one or more user affordances for providing at least a corresponding margin status of the respective tissue section, where the displaying further comprises displaying the customizable user interface at the second electronic device, responsive to a first user interaction with respect to the customizable user interface at the first electronic device, the first user interaction is displayed on the customizable user interface at the second electronic device, and responsive to a second user interaction with respect to the customizable user interface at the second electronic device, the second user interaction is displayed on the customizable user interface at the first electronic device; receiving, from the second electronic device, a second plurality of annotations to the first three-dimensional representation of the tumor resection defect, where the second plurality of annotations comprises, for each respective tissue section in the corresponding plurality of tissue sections that satisfies a first margin status criterion based on the corresponding margin status, an indication of one or more supplemental specimens obtained from the tumor resection defect that map to the respective tissue section; and generating a report for the subject using, at least, the first three-dimensional representation of the tumor specimen, the first plurality of annotations, the first three-dimensional representation of the tumor resection defect, the second plurality of annotations, and the customizable user interface.

Another aspect of the present disclosure provides a method for localizing close or positive margins in a tumor specimen from a subject. In some embodiments, the method is performed at a first electronic device with a display and an input device. In some embodiments, the method includes obtaining a first three-dimensional representation of the tumor specimen. In some embodiments, the three-dimensional representation comprises more than 1000 three-dimensional points that define a boundary of the tumor specimen. A first plurality of annotations to the first three-dimensional representation of the tumor specimen is received. The first plurality of annotations comprises a first subset of annotations, each respective annotation in the first subset of annotations indicating an orientation of the first three-dimensional representation of the tumor specimen relative to the subject, a second subset of annotations, each respective annotation in the second subset of annotations indicating a respective tissue section in a corresponding plurality of tissue sections from the tumor specimen, and a third subset of annotations, each respective annotation in the third subset of annotations providing a corresponding margin status for a respective tissue section in the plurality of tissue sections, where the corresponding margin status indicates whether the respective tissue section has a close or positive margin based upon a pathological analysis of the respective tissue section. A composite three-dimensional representation of the tumor specimen is formed by modifying the first three-dimensional representation of the tumor specimen to include the first plurality of annotations.

In some embodiments, the tumor specimen is from a cancer selected from the group consisting of adrenal cancer, biliary tract cancer, bladder cancer, bone/bone marrow cancer, brain cancer, breast cancer, cervical cancer, colorectal cancer, cancer of the esophagus, gastric cancer, head/neck cancer, hepatobiliary cancer, kidney cancer, liver cancer, lung cancer, ovarian cancer, pancreatic cancer, pelvis cancer, pleura cancer, prostate cancer, renal cancer, skin cancer, stomach cancer, testis cancer, thymus cancer, thyroid cancer, uterine cancer, lymphoma, melanoma, multiple myeloma, and leukemia.

In some embodiments, the first three-dimensional representation of the tumor specimen is obtained by a procedure comprising receiving a first partial representation of the tumor specimen comprising at least a 330° scan of the tumor specimen at a corresponding first orientation; receiving a second partial representation of the tumor specimen comprising at least a 330° scan of the tumor specimen at a corresponding second orientation, where the second partial representation at the corresponding second orientation captures a region of the tumor specimen that is obscured at the first orientation; and determining a first plurality of landmarks, where each respective landmark in the first plurality of landmarks comprises a corresponding pair of reference positions including a first respective reference position for the first partial representation and a second respective reference position for the second partial representation, where the first and second reference positions are positioned at a common feature visible in the first and second partial representations. Thus, a first set of landmark coordinates for the first partial representation and a second set of landmark coordinates for the second partial representation are identified. The procedure further includes using the first set of landmark coordinates for the first partial representation and the second set of landmark coordinates for the second partial representation to obtain a first alignment of the first partial representation with the second partial representation.

In some embodiments, the method further includes, responsive to user input, turning the composite three-dimensional representation in three-dimensional space, where the receiving the first plurality of annotations to the first three-dimensional representation of the tumor specimen comprises performing a manual electronic annotation of the first three-dimensional representation, for each respective annotation in the first plurality of annotations, to produce the composite three-dimensional representation.

In some embodiments, the method further includes, responsive to a user interaction, performing an action on all or a portion of the composite three-dimensional representation selected from the group consisting of: changing a scale of the composite three-dimensional representation, panning the composite three-dimensional representation, rotating the composite three-dimensional representation, and highlighting a user selected portion of the composite three-dimensional representation.

In some embodiments, the method further includes determining a corresponding margin status for a respective tissue section in the plurality of tissue sections using frozen section analysis, where each respective tissue section in the plurality of tissue sections is a frozen tissue section.

In some embodiments, a first portion of the tumor specimen comprises cancerous tissue, a second portion of the tumor specimen comprises normal tissue, and the corresponding margin status for a respective tissue section indicates that the respective tissue section has a close margin when a distance between a leading edge of the cancerous tissue and an outer surface of the tumor specimen satisfies a first distance criterion. In some embodiments, the distance between a leading edge of the cancerous tissue and an outer surface of the tumor specimen satisfies the first distance criterion when the distance is between a first threshold distance and a second threshold distance that is greater than the first threshold distance, where the first threshold distance is at least about 1 mm, at least about 2 mm, or at least about 3 mm, and the second threshold distance is no more than about 6 mm, no more than about 5 mm, or no more than about 4 mm.

In some embodiments, a first portion of the tumor specimen comprises cancerous tissue, a second portion of the tumor specimen comprises normal tissue, and the corresponding margin status for a respective tissue section indicates that the respective tissue section has a positive margin when a distance between a leading edge of the cancerous tissue and an outer surface of the tumor specimen satisfies a second distance criterion. In some embodiments, the distance between a leading edge of the cancerous tissue and an outer surface of the tumor specimen satisfies the second distance criterion when the distance is less than a third threshold distance, where the third threshold distance is no more than about 3 mm, no more than about 2 mm, or no more than about 1 mm.

In some embodiments, the corresponding margin status further indicates whether the respective tissue section has a negative margin based upon the pathological analysis of the respective tissue section. In some embodiments, a first portion of the tumor specimen comprises cancerous tissue, a second portion of the tumor specimen comprises normal tissue, and the corresponding margin status for a respective tissue section indicates that the respective tissue section has a negative margin when a distance between a leading edge of the cancerous tissue and an outer surface of the tumor specimen satisfies a third distance criterion. In some embodiments, the distance between a leading edge of the cancerous tissue and an outer surface of the tumor specimen satisfies the third distance criterion when the distance is less than a fourth threshold distance, where the fourth threshold distance is at least about 4 mm, at least about 5 mm, or at least about 6 mm.

In some embodiments, each respective annotation in the first subset of annotations is selected from the group consisting of: anterior, posterior, lateral, medial, right, left, superior, and inferior.

In some embodiments, the method further includes communicating one or more composite three-dimensional representation instructions to a remote electronic device where the one or more composite three-dimensional representation instructions comprises (i) instructions for displaying an image overlay on the remote electronic device comprising the composite three-dimensional representation in the form of the first plurality of annotations and the first three-dimensional representation of the tumor specimen, thereby producing a second three-dimensional representation of the tumor specimen, and (ii) instructions that, responsive to a first user interaction with respect to the composite three-dimensional representation of the tumor specimen at the first electronic device, apply the first user interaction to the second three-dimensional representation of the tumor specimen at the remote electronic device.

In some embodiments, the first user interaction is selected from the group consisting of changing a scale of the composite three-dimensional representation, panning the composite three-dimensional representation, rotating composite three-dimensional representation, and highlighting a user selected portion of the composite three-dimensional representation.

In some embodiments, the first user interaction comprises adding one or more annotations to the first three-dimensional representation of the tumor specimen.

In some embodiments, the first electronic device and the remote electronic device are located at different facilities. In some embodiments, the first electronic device is located in a frozen section (FS) laboratory, and the remote electronic device is located in an operating room (OR).

In some embodiments, the first user interaction is performed using a mouse or a stylus.

In some embodiments, the method further includes obtaining a first three-dimensional representation of a tumor resection defect in the subject corresponding to the tumor specimen (e.g., where the first three-dimensional representation of the corresponding tumor resection defect comprises more than 1000 three-dimensional points that define a boundary of the tumor resection defect). In some embodiments, the obtaining the first three-dimensional representation of the corresponding tumor resection defect in the subject further comprises determining a completion status of the first three-dimensional representation of the corresponding tumor resection defect as complete or incomplete, and when the completion status of the first three-dimensional representation of the corresponding tumor resection defect is incomplete, receiving a reference representation of a defect, and mapping the first three-dimensional representation of the corresponding tumor resection defect to the reference representation of the defect, thereby correcting one or more incomplete portions of the first three-dimensional representation of the corresponding tumor resection defect.

In some embodiments, the method further includes, when a corresponding margin status of a respective tissue section satisfies a first margin status criterion, obtaining one or more supplemental specimens from the subject at a harvesting location in the tumor resection defect that is adjacent to an originating location of the respective tissue section in the tumor specimen, and receiving a second plurality of annotations to the first three-dimensional representation of the tumor resection defect, the second plurality of annotations comprising, for each supplemental specimen in the one or more supplemental specimens, a corresponding mapping of the respective supplemental specimen to the tumor resection defect.

In some embodiments, the first margin status criterion is satisfied when the respective tissue section has a close or positive margin based upon the pathological analysis of the respective tissue section.

In some embodiments, the first three-dimensional representation of the corresponding tumor resection defect is received from a remote electronic device, and the method further includes displaying, after the receiving, at the first electronic device, a second three-dimensional representation of the corresponding tumor resection defect, and applying, responsive to a first user interaction with respect to the first three-dimensional representation of the corresponding tumor resection defect at the remote electronic device, the first user interaction to the second three-dimensional representation of the corresponding tumor resection defect at the first electronic device.

In some embodiments, the second three-dimensional representation of the corresponding tumor resection defect is a composite three-dimensional representation of the tumor resection defect comprising the first three-dimensional representation of the tumor resection defect modified to include the second plurality of annotations.

In some embodiments, the method further includes displaying, on the display of the first electronic device, a customizable user interface comprising, for each respective tissue section in the corresponding plurality of tissue sections from the tumor specimen, a first affordance for providing a first annotation that indicates an identity of the tumor specimen, a second affordance for providing a second annotation that indicates an identity of the respective tissue section, a third affordance for providing a third annotation that provides a corresponding margin status for the respective tissue section, and a fourth affordance for updating a review status of the respective tissue section.

In some embodiments, the method further includes, upon user selection of the fourth affordance, updating the review status of the respective tissue section from a first review status to a second review status.

In some embodiments, the customizable user interface further comprises a fifth affordance for appending one or more supplemental specimens to the plurality of tissue sections. In some embodiments, the method further includes, when a corresponding margin status for a respective tissue section in the plurality of tissue sections satisfies a first margin status criterion, obtaining one or more supplemental specimens from the subject at a location in the tumor resection defect that is adjacent to an originating location of the respective tissue section, appending, upon user selection of the fifth affordance, the one or more supplemental specimens to the plurality of tissue sections, updating, upon user selection of the fourth affordance, the review status of the respective tissue section from a first review status to a second review status, and, for each respective supplemental specimen in the one or more supplemental specimens, indicating, upon user selection of the second affordance, (i) an identity of the respective supplemental specimen and (ii) an identity of the respective tissue section in the plurality of tissue sections.

In some embodiments, the method further includes communicating one or more user interface instructions to a remote electronic device, where the one or more user interface instructions comprises (i) instructions for displaying an image overlay on the remote electronic device comprising the customizable user interface, and (ii) instructions that, responsive to a first user interaction with respect to the customizable user interface at the first electronic device, apply the first user interaction to the customizable user interface at the remote electronic device.

In some embodiments, the method further includes generating a report for the subject using at least the first three-dimensional representation of the tumor specimen and the plurality of annotations. In some embodiments, the report is an electronic health record (EHR).

Another aspect of the present disclosure provides an electronic device, comprising a display, an input device, one or more processors, memory, and one or more programs, where the one or more programs are stored in the memory and configured to be executed by the one or more processors, the one or more programs including instructions for a method for localizing close or positive margins in a tumor specimen from a subject. In some embodiments, the method includes obtaining a first three-dimensional representation of the tumor specimen. In some embodiments, the three-dimensional representation comprises more than 1000 three-dimensional points that define a boundary of the tumor specimen. A first plurality of annotations to the first three-dimensional representation of the tumor specimen is received. The first plurality of annotations comprises a first subset of annotations, each respective annotation in the first subset of annotations indicating an orientation of the first three-dimensional representation of the tumor specimen relative to the subject, a second subset of annotations, each respective annotation in the second subset of annotations indicating a respective tissue section in a corresponding plurality of tissue sections from the tumor specimen, and a third subset of annotations, each respective annotation in the third subset of annotations providing a corresponding margin status for a respective tissue section in the plurality of tissue sections, where the corresponding margin status indicates whether the respective tissue section has a close or positive margin based upon a pathological analysis of the respective tissue section. A composite three-dimensional representation of the tumor specimen is formed by modifying the first three-dimensional representation of the tumor specimen to include the first plurality of annotations.

Another aspect of the present disclosure provides a non-transitory computer-readable storage medium having stored thereon program code instructions that, when executed by a processor, cause the processor to perform a method for localizing close or positive margins in a tumor specimen from a subject. In some embodiments, the method includes obtaining a first three-dimensional representation of the tumor specimen. In some embodiments, the three-dimensional representation comprises more than 1000 three-dimensional points that define a boundary of the tumor specimen. A first plurality of annotations to the first three-dimensional representation of the tumor specimen is received. The first plurality of annotations comprises a first subset of annotations, each respective annotation in the first subset of annotations indicating an orientation of the first three-dimensional representation of the tumor specimen relative to the subject, a second subset of annotations, each respective annotation in the second subset of annotations indicating a respective tissue section in a corresponding plurality of tissue sections from the tumor specimen, and a third subset of annotations, each respective annotation in the third subset of annotations providing a corresponding margin status for a respective tissue section in the plurality of tissue sections, where the corresponding margin status indicates whether the respective tissue section has a close or positive margin based upon a pathological analysis of the respective tissue section. A composite three-dimensional representation of the tumor specimen is formed by modifying the first three-dimensional representation of the tumor specimen to include the first plurality of annotations.

Still another aspect of the present disclosure provides a method for localizing close or positive margins in a tumor specimen from a subject. In some embodiments, the method is performed at a first electronic device with a display and an input device. In some embodiments, the method includes receiving, from a second electronic device (i) a first three-dimensional representation of the tumor specimen. In some such embodiments, the first three-dimensional representation comprises more than 1000 three-dimensional points that define a boundary of the tumor specimen. In some embodiments, there is further received (ii) a first plurality of annotations to the first three-dimensional representation of the tumor specimen, the first plurality of annotations comprising a first subset of annotations, each respective annotation in the first subset of annotations indicating an orientation of the first three-dimensional representation of the tumor specimen relative to the subject; a second subset of annotations, each respective annotation in the second subset of annotations indicating a respective tissue section in a corresponding plurality of tissue sections from the tumor specimen; and a third subset of annotations, each respective annotation in the third subset of annotations providing a corresponding margin status for a respective tissue section in the plurality of tissue sections, where the corresponding margin status indicates whether the respective tissue section has a close or positive margin based upon a pathological analysis of the respective tissue section. In some embodiments, there is further received (iii) one or more composite three-dimensional representation instructions. In some embodiments, the method further includes displaying, after the receiving (i) a composite three-dimensional representation of the tumor specimen in the form of the first plurality of annotations and the first three-dimensional representation of the tumor specimen, thereby producing a second three-dimensional representation of the tumor specimen, and (ii) responsive to a user interaction with respect to the composite three-dimensional representation of the tumor specimen at the second electronic device, the user interaction applied to the second three-dimensional representation of the tumor specimen.

In some embodiments, the method further includes, at the first electronic device, obtaining a first three-dimensional representation of a corresponding tumor resection defect. One or more tumor resection defect instructions are communicated to the second electronic device, where the one or more tumor resection defect instructions comprises (i) instructions for displaying an image overlay on the second electronic device comprising the first three-dimensional representation of the corresponding tumor resection defect, thereby producing a second three-dimensional representation of the corresponding tumor resection defect, and (ii) instructions that, responsive to a first user interaction with respect to the three-dimensional representation of the corresponding tumor resection defect at the first electronic device, apply the first user interaction to the second three-dimensional representation of the corresponding tumor resection defect at the second electronic device.

In some embodiments, the method further includes, when a corresponding margin status of a respective tissue section satisfies a first margin status criterion, obtaining one or more supplemental specimens from the subject at a harvesting location in the tumor resection defect that is adjacent to an originating location of the respective tissue section in the tumor specimen, and receiving a second plurality of annotations to the first three-dimensional representation of the tumor resection defect. In some embodiments, the second plurality of annotations comprises, for each supplemental specimen in the one or more supplemental specimens, a corresponding mapping of the respective supplemental specimen to the tumor resection defect.

In some embodiments, the second three-dimensional representation of the corresponding tumor resection defect on the second electronic device is a composite three-dimensional representation of the tumor resection defect comprising the first three-dimensional representation of the tumor resection defect modified to include the second plurality of annotations.

In some embodiments, the method further includes displaying, on the display of the first electronic device, a customizable user interface comprising, for each respective tissue section in the corresponding plurality of tissue sections from the tumor specimen, a first affordance for providing a first annotation that indicates an identity of the tumor specimen, a second affordance for providing a second annotation that indicates an identity of the respective tissue section, a third affordance for providing a third annotation that provides a corresponding margin status for the respective tissue section, and a fourth affordance for updating a review status of the respective tissue section.

In some embodiments, the method further includes communicating one or more user interface instructions to the second electronic device, where the one or more user interface instructions comprises (i) instructions for displaying an image overlay on the remote electronic device comprising the customizable user interface, and (ii) instructions that, responsive to a first user interaction with respect to the customizable user interface at the first electronic device, apply the first user interaction to the customizable user interface at the second electronic device.

Yet another aspect of the present disclosure provides a method for localizing a tissue section in a tumor specimen from a subject to a corresponding tumor resection defect in the subject. In some embodiments, the method is performed at an electronic device with a display and an input device. In some embodiments, the method includes obtaining a first three-dimensional representation of the tumor specimen. In some embodiments, the three-dimensional representation comprises more than 1000 three-dimensional points that define a boundary of the tumor specimen. A first plurality of annotations to the first three-dimensional representation of the tumor specimen is received, the first plurality of annotations comprising a first subset of annotations, each respective annotation in the first subset of annotations indicating an orientation of the first three-dimensional representation of the tumor specimen relative to the subject, a second subset of annotations, each respective annotation in the second subset of annotations indicating a respective tissue section in a corresponding plurality of tissue sections from the tumor specimen, and a third subset of annotations, each respective annotation in the third subset of annotations providing a corresponding margin status for a respective tissue section in the plurality of tissue sections, where the corresponding margin status indicates whether the respective tissue section has a close or positive margin based upon a pathological analysis of the respective tissue section. A first three-dimensional representation of a corresponding tumor resection defect in the subject is obtained. In some embodiments, the first three-dimensional representation of the corresponding tumor resection defect comprises more than 1000 three-dimensional points that define a boundary of the tumor resection defect. In some embodiments, the method further includes determining an orientation of the tumor specimen relative to the corresponding tumor resection defect, and applying, when a corresponding margin status of a respective tissue section in the plurality of tissue sections satisfies a first margin status criterion, a second plurality of annotations to the first three-dimensional representation of the tumor resection defect. In some embodiments, the second plurality of annotations comprises, for each respective supplemental specimen in one or more supplemental specimens associated with the respective tissue section, a corresponding mapping of the respective supplemental specimen to the tumor resection defect.

In some embodiments, the one or more supplemental specimens are obtained from the subject at a harvesting location in the tumor resection defect that is adjacent to an originating location of the respective tissue section in the tumor specimen.

Yet another aspect of the present disclosure provides an electronic device, comprising a display, an input device, one or more processors, memory, and one or more programs, where the one or more programs are stored in the memory and configured to be executed by the one or more processors, the one or more programs including instructions for performing any of the methods disclosed herein.

Still another aspect of the present disclosure provides a non-transitory computer-readable storage medium having stored thereon program code instructions that, when executed by a processor, cause the processor to perform any of the methods disclosed herein.

### INCORPORATION BY REFERENCE

All publications, patents, and patent applications herein are incorporated by reference in their entireties. In the event of a conflict between a term herein and a term in an incorporated reference, the term herein controls.

### BRIEF DESCRIPTION OF THE FIGURES

FIGS. 1A and 1B illustrate a schematic of an equipment setup, in accordance with some embodiments of the present disclosure. In FIG. 1A, a 3D scanner is depicted on the left with a turntable on the right. In FIG. 1B, the setup is depicted with a tumor specimen placed onto the turntable. In some embodiments, the equipment setup further includes a computer system from which associated 3D graphics software can be accessed.

FIG. 2 illustrates an example of a point annotation, in accordance with some embodiments of the present disclosure. The arrow indicates the location of the annotated point, while the text is “attached” to the point annotation.

FIG. 3 illustrates an example virtual three-dimensional (3D) specimen map of a composite resection including segmental mandibulectomy and total glossectomy, in accordance with an embodiment of the present disclosure. The site of each frozen section (lines 302) is drawn directly onto the specimen using virtual annotation tools, and positive margin results are indicated accordingly. The virtual 3D specimen map is displayed in both the pathology lab and operating room in real time. The left panel illustrates a right lateral view. The right panel illustrates a superior view.

FIG. 4 illustrates an example flowchart comparing the integration of an operational protocol in accordance with some embodiments of the present disclosure (white text boxes) alongside the traditional frozen section process (black text boxes). Gray text boxes indicates steps followed by both the presently disclosed approach and the traditional approach. OR indicates operating room; FS indicates frozen section.

FIGS. 5A, 5B, and 5C collectively illustrate a surgical specimen and 3D representation and annotations thereof, in accordance with an embodiment of the present disclosure. FIG. 5A depicts a gross anterior mandible specimen. FIG. 5B shows pre-rendered scans of the anterior and posterior sides. The virtual markers 502, 504, and 506 were selected by the operator to indicate common points shared between the two scans. FIG. 5C shows a single model in which the two scans were aligned using the EinScan program. The single model was then virtually annotated by the pathologist with a digital marker (shown as white annotations) to indicate sampled sections.

FIGS. 6A, 6B, 6C, and 6D collectively illustrate a surgical specimen and 3D representation and annotations thereof, in accordance with an embodiment of the present disclosure. FIG. 6A illustrates a gross specimen from a composite resection including a partial glossectomy and segmental mandibulectomy. This specimen greatly exceeds the minimum size threshold for an ideal scan. FIG. 6B depicts a crude, hand-drawn two-dimensional (2D) specimen map created by the pathologist on site. FIGS. 6C-D show various views of a 3D annotated scan of the mandibulectomy specimen with anatomical landmarks labeled (FIG. 6C) and numbered margin slices (FIG. 6D). The comparison of the 3D images (FIGS. 6C-D) and the traditional hand drawn images (FIG. 6B) created by the pathologist clearly demonstrates the superiority of the presently disclosed systems and methods.

FIG. 7 illustrates a composite resection of mandible and floor of mouth (right), in accordance with an embodiment of the present disclosure. Note the bone of the mandible has been highlighted using white shading (indicated by the black arrows) in Microsoft Paint 3D to serve as a visual landmark. Histopathological margin results (left) were later notated within the modeling application for permanent documentation.

FIGS. 8A and 8B collectively illustrate a surgical specimen and 3D representation and annotations thereof, in accordance with an embodiment of the present disclosure. FIG. 8A depicts a gross radical tonsillectomy specimen. This specimen exemplifies smaller specimens that approach the minimum size threshold. FIG. 8B shows a 3D annotated scan of the tonsillectomy specimen with anatomic orientation labeled and numbered serial sampling sections. White asterisks denote positive margins, whereas black asterisks denote margins equivalent to or greater than 5 mm. Darker shading (indicated by areas 802) was used to highlight areas requiring additional supplemental margins.

FIGS. 9A, 9B, 9C, and 9D collectively illustrate a surgical specimen and 3D representation and annotations thereof, in accordance with an embodiment of the present disclosure. FIGS. 9A-B illustrate a gross palatomaxillectomy specimen. This specimen serves as an ideal example of contrast between various tissue components of the specimen. FIGS. 9C-D depict various views of a 3D annotated scan of the palatal specimen with anatomical landmarks labeled and numbered margin slices.

FIGS. 10A, 10B, 10C, and 10D collectively illustrate a surgical specimen and 3D representation and annotations thereof, in accordance with an embodiment of the present disclosure. FIGS. 10A-B depict a gross laryngeal specimen with appreciable textural complexity. FIGS. 10C-D show a 3D annotated scan of the laryngeal specimen with anatomical landmarks labeled and margin slices numbered.

FIGS. 11A and 11B illustrate various views of a radical right oropharyngectomy specimen resected via transoral robotic surgery (TORS), in accordance with an embodiment of the present disclosure. FIG. 11C shows a 3D scan of the tonsil specimen, in accordance with an embodiment of the present disclosure. The pins marked as 1102, 1104, and 1106 serve as external fiduciaries, identifying critical anatomic landmarks within the patient (1102=superior, 1104=lateral, 1106=medial).

FIG. 12A illustrates a gross palatal specimen with teeth serving as natural fiducial landmarks for alignment and orientation, in accordance with an embodiment of the present disclosure. FIG. 12B shows a 3D annotated scan of the palatal specimen with numbered margin slices, in accordance with an embodiment of the present disclosure.

FIG. 13A shows a gross facial skin specimen with exophytic tumor, in accordance with an embodiment of the present disclosure. FIG. 13B illustrates a 3D scan of the facial skin specimen, in accordance with an embodiment of the present disclosure. Margins were not annotated, as an in-person consultation was ultimately required. The software generates a bright highlighted texture (indicated by the black arrow) to highlight regions of the model that could not be rendered accurately. This 3D scan demonstrates the challenges associated with scanning specimens that are exclusively soft tissue and oddly shaped.

FIG. 14 illustrates an example schematic of a tumor specimen removed from a subject (top left) and a corresponding tumor resection defect in the subject (top right), in accordance with some embodiments of the present disclosure. Surgical specimens removed from the patient can be annotated according to their gross anatomical orientation. Removal of a tumor specimen from a patient results in a change of the coordinate space (e.g., translation, rotation, and shearing) resulting in a “specimen-space” (bottom left) and “patient-space” (bottom right) which are out of alignment. The change in orientation from landmarks within the patient to landmarks within the resected specimen can be mathematically modeled to provide clear instructions to the surgeon regarding exact locations of margins of concern.

FIG. 15 illustrates an example of 3D models of a specimen (left) and hypothetical defect (right), in accordance with an embodiment of the present disclosure. Because they are scanned at different times and places, and because of deformations in both structures following surgery, elastic registration is used to map the contour of the specimen surface into the matching location of the defect.

FIG. 16 illustrates an example of the specimen (left) and simulated defect (right) mesh clouds, with point annotations (black arrows) dropped on corresponding locations, in accordance with an embodiment of the present disclosure. These annotations allow the physician to orient the annotations on the specimen in the context of the patient anatomy (represented by the surface of the right-hand structure).

FIGS. 17A, 17B, and 17C collectively illustrate an example of a customizable user interface, in accordance with some embodiments of the present disclosure.

FIGS. 18A and 18B collectively illustrate an example block diagram illustrating a computing device in accordance with some embodiments of the present disclosure.

FIGS. 19A, 19B, 19C, and 19D collectively illustrate an example flow chart of a method for localizing close or positive margins in a tumor specimen from a subject, where optional steps are indicated by dashed lines, in accordance with some embodiments of the present disclosure.

FIG. 20 illustrates an example workflow for the use of three-dimensional representations of a tumor specimen and a tumor resection defect to enhance communication and documentation of tissue sections from the tumor specimen, in accordance with an embodiment of the present disclosure.

### DETAILED DESCRIPTION

### Introduction

In some cases, patients with low-stage head and neck malignancies are treated with only primary surgery. The therapeutic goal in such scenarios is to reduce the risk of additive toxicity of adjuvant radiotherapy by ensuring resection margin adequacy (e.g., “clean margins”). Alternatively, patients with high-stage head and neck neoplasia can be treated with primary surgery plus planned postoperative chemoradiation. However, adjuvant radiotherapy is not effective in “sterilizing” inadequate resection margins. Thus, adequate margins are critical to the successful treatment of most head and neck malignancies.

The literature is replete with data supporting the importance of margin adequacy [1]-[4]. In some example implementations, resection margin status is determined during surgery and dictates the extent of resection. While the patient is under anesthesia, the resected tumor is brought from the operating room (OR) to the pathology suite, where it is dissected, annotated, and/or histologically examined for resection margin status. During this time, the surgeon scrubs out of the OR, physically walks to the pathology lab, and orients the specimen with the surgical pathologist. If margins are inadequate (e.g., <5 mm), the pathologist communicates to the surgeon the exact location of region(s) of concern. This can be simple for small tumors (e.g., “the floor of mouth region”), but communicating the locations of regions of concern is more challenging for larger or more complex specimens. The surgeon may scrub out again during surgery and return to the pathology suite. At this point, the annotated (e.g., painted) and dissected resection specimen bears no resemblance to the original specimen, and both surgeon and pathologist are deeply frustrated at the communication gap. Then the surgeon must scrub back into the OR, and translate what the pathologist attempts to convey into the action of harvesting supplemental resection margins.

At this point in the process, new opportunities for miscommunications arise, such as whether the supplemental margins harvested and sent to frozen section actually correspond to the margin planes of concern. For instance, in some cases, the labels identifying supplemental margins (e.g., supplemental tongue base) cannot truly document their anatomic extent. While a surgeon might include a drawing as reference, potential issues remain in that, for instance, at a multidisciplinary tumor board and/or at any downstream intervention, the documentation allows only for matching up the names from supplemental margins with the names of resection margin planes from the initial margin assessment. If a subsequent medical provider, such as a treating radiation oncologist, is at another institution, he or she must piece together the operating room report with the pathology report in an attempt to gain understanding of regions requiring greater dosing. Accurate and efficient pathologist/surgeon communication can minimize the waste of precious surgical suite resources.

Importantly, surgeons scrubbing out one or more times during surgery increases anesthesia duration. Published data support the idea that increased anesthesia duration increases the rates of complications, and thus minimizing excess anesthesia time is a worthy goal. In 2018, Cheng and colleagues published a systematic review and meta-analysis based on 59 retrospective and 7 prospective studies encompassing all surgeries with a total patient cohort of 105,648 [5]. Pooled analyses demonstrated significantly increased likelihood of major postoperative complications with increased anesthesia duration. Every 30 minutes anesthesia duration, after the first two hours of surgery, is associated with a 14% increase in major complication likelihood (e.g., Grade III-V surgical complications, surgical site infections, sepsis). Specifically regarding head and neck cancer surgeries, the development of microvascular reconstructive techniques over the last 50 years has greatly improved the oncologic quality of resections and patient quality of life. Brady and colleagues demonstrated a direct relationship between complications and anesthesia duration in a retrospective study of 630 patients requiring free-flap reconstruction [6]. Multivariate analysis was performed, adjusting for age, sex, race, and obesity; the first quintile group served as reference (mean operative time of 400 minutes+/−123.8). They demonstrated that the fifth quintile group (mean operative time 817.0 minutes+/−118.7) had increased risk of overall complications (odds ratio (OR) 1.98, 95% CI: 1.10-3.58, p=0.02), surgical complications (OR 2.46, 95% CI: 1.35-4.46, p=0.003), and postoperative transfusions (OR 2.31, 95% CI: 1.27-4.20, p=0.006).

Thus, in some embodiments, communication gaps waste operating room (OR) time, increase anesthesia duration increasing the risk to patients, and/or leave holes in medical documentation.

Cryostat sections stained with hematoxylin and eosin remain the gold standard for intraoperative cancer confirmation and margin assessment since the early 20^{th} century [7]. Fluorophores are FDA-approved molecules with emission peaks in the near infrared range widely used in medical imaging either alone or conjugated to monoclonal antibodies [8]. Early clinical trials with fluorophore-tagged EGFR antibodies (Cetuximab-IRDye800, NCT019873750 and Pantimumab-IRDye800, NCT02415881) demonstrate that fluorescence intensity correlates with resection margin distance for squamous carcinoma head and neck resections with high sensitivity and specificity [9]. This approach introduces a cancer detection step with the potential to guide surgeons regarding the extent of surgery, and guide pathologists as to where to commence margin mapping. But this approach is still in early development and larger multicenter studies are needed. Intraoperative ultrasound analysis is another approach which could potentially guide surgeons regarding the extent of surgery [10], but does not address the communication and documentation gaps identified above.

With the advent of low-cost, high-resolution 3D surface scanners, it is possible to enhance this communication pipeline to reduce OR and anesthesia time, improve communication clarity, and achieve better precision when describing margin status in the context of patient anatomy. Unfortunately, the existing software for viewing and annotating model obtained from 3D scanners is insufficient. For instance, current tools do not allow for labels in space to follow the specimen correctly as it rotates. This necessitates a more pain-staking approach of writing directly on the specimen. Annotations cannot be exported to an easily transportable format, like plain-text JavaScript Object Notation (JSON) files, which can be imported to reporting software. In some instances, the laterality of the specimen is also not supported, which can result in the misidentification of orientation annotations (e.g., right and left in the anterior view).

Conventional methods do not provide a formal system for acquiring, annotating, or describing this data. Moreover, there is no established protocol for using this data in communication of surgical margins or gross anatomical positioning for tumor resections. Accordingly, the systems and methods described in the present disclosure can beneficially lead to widespread adoption of these inexpensive tools and methods, improving the quality of surgeon/pathologist communication and documentation of the final resection specimen margin status, and decreasing anesthesia duration.

Unlike the developing approaches described above, which help identify margin status and guide surgical resection, the presently disclosed systems and methods are based on facilitating communication between physicians using inexpensive hardware and custom-developed software. The scanning tools are already commercially available, but require specially-designed software for annotation and analysis of 3D models and annotations. Because of these advantages, the system proposed here can be deployed immediately following development, as it does not rely on new chemical or physical processes. Further, beyond the purchase of 3D scanners and software, the protocol described in the present disclosure does not require additional reagents beyond what is required of typical surgical intervention. Patients with higher T stage cancers, or specimens from more anatomically complex regions are most likely to benefit from this protocol. This is even more crucial in salvage surgery for recurrent disease; inadequate resection margins are even less acceptable if adjuvant radiotherapy is not an option.

Accordingly, the present disclosure provides systems and methods for localizing close or positive margins in a tumor specimen from a subject. A three-dimensional representation of the specimen is obtained (e.g., where the representation comprises more than 1000 three-dimensional points that define a boundary of the specimen). A plurality of annotations to the representation is received, including a first subset of annotations indicating an orientation of the representation relative to the subject, a second subset of annotations indicating a plurality of tissue sections from the specimen, and a third subset of annotations providing a margin status for respective tissue sections in the plurality of tissue sections, where the margin status indicates close or positive margins based upon pathological analysis of the respective tissue sections. A composite three-dimensional representation of the tumor specimen is formed by modifying the first three-dimensional representation of the tumor specimen to include the plurality of annotations.

### REFERENCES

[1] T. Y. Chen, L. J. Emrich, and D. L. Driscoll, “The clinical significance of pathological findings in surgically resected margins of the primary tumor in head and neck carcinoma,” Int. J. Radiat. Oncol. Biol. Phys., vol. 13, no. 6, pp. 833-837, June 1987, doi: 10.1016/0360-3016 (87) 90095-2.
[2] T. R. Loree and E. W. Strong, “Significance of positive margins in oral cavity squamous carcinoma,” Am. J. Surg., vol. 160, no. 4, pp. 410-414, October 1990, doi: 10.1016/s0002-9610 (05) 80555-0.
[3] G. El-Husseiny et al., “Squamous cell carcinoma of the oral tongue: an analysis of prognostic factors,” Br. J. Oral Maxillofac. Surg., vol. 38, no. 3, pp. 193-199, June 2000, doi: 10.1054/bjom. 1999.0235.
[4] P. Garzino-Demo et al., “Clinicopathological parameters and outcome of 245 patients operated for oral squamous cell carcinoma,” J. Cranio-Maxillo-fac. Surg. Off. Publ. Eur. Assoc. Cranio-Maxillo-fac. Surg., vol. 34, no. 6, pp. 344-350, September 2006, doi: 10.1016/j.jcms.2006.04.004.
[5] H. Cheng et al., “Prolonged operative duration is associated with complications: a systematic review and meta-analysis,” J. Surg. Res., vol. 229, pp. 134-144, September 2018, doi: 10.1016/j.jss.2018.03.022.
[6] J. S. Brady et al., “Association of Anesthesia Duration with Complications After Microvascular Reconstruction of the Head and Neck,” JAMA Facial Plast. Surg., vol. 20, no. 3, pp. 188-195, May 2018, doi: 10.1001/jamafacial.2017.1607.
[7] A. A. Gal and P. T. Cagle, “The 100-year anniversary of the description of the frozen section procedure,” JAMA, vol. 294, no. 24, pp. 3135-3137 Dec. 2005, doi: 10.1001/jama.294.24.3135.
[8] Y.-J. Lee et al., “Intraoperative Fluorescence-Guided Surgery in Head and Neck Squamous Cell Carcinoma,” The Laryngoscope, vol. 131, no. 3, pp. 529-534, 2021, doi: 10.1002/lary.28822.
[9] E. L. Rosenthal et al., “Safety and Tumor Specificity of Cetuximab-IRDye800 for Surgical Navigation in Head and Neck Cancer,” Clin. Cancer Res. Off. J. Am. Assoc. Cancer Res., vol. 21, no. 16, pp. 3658-3666 Aug. 2015, doi: 10.1158/1078-0432.CCR-14-3284.
[10] O. Tarabichi, V. Kanumuri, A. F. Juliano, W. C. Faquin, M. E. Cunnane, and M. A. Varvares, “Intraoperative Ultrasound in Oral Tongue Cancer Resection: Feasibility Study and Early Outcomes,” Otolaryngol.—Head Neck Surg. Off. J. Am. Acad. Otolaryngol.—Head Neck Surg., vol. 158, no. 4, pp. 645-648, April 2018, doi: 10.1177/0194599817742856.

Unless defined otherwise, all technical and scientific terms used herein have the same meaning as is commonly understood by one of skill in the art to which this disclosure belongs. All patents and publications referred to herein are incorporated by reference in their entireties.

### Definitions

As used interchangeably herein, the term “specimen” or “sample” refers to a sample obtained or derived from a source of interest, as described herein. In some embodiments, a specimen or sample is a biological specimen or a biological sample. In certain embodiments, a source of interest comprises an organism, such as an animal or human. In certain embodiments, a sample is a biological tissue or fluid. In some embodiments, a specimen or sample is a liquid biopsy or a solid tissue biopsy. Non-limiting examples of samples include bone marrow, blood, blood cells, ascites, (tissue or fine needle) biopsy samples, cell-containing body fluids, free floating nucleic acids, sputum, saliva, urine, cerebrospinal fluid, peritoneal fluid, pleural fluid, feces, lymph, gynecological fluids, swabs (e.g., skin swabs, vaginal swabs, oral swabs, and nasal swabs), washings or lavages such as a ductal lavages or broncheoalveolar lavages, aspirates, scrapings, specimens (e.g., bone marrow specimens, tissue biopsy specimens, and surgical specimens), feces, other body fluids, secretions, and/or excretions, and cells therefrom, etc.

As used herein, the term “subject” refers to any animal (e.g., a mammal), including, but not limited to, humans, and non-human animals (including, but not limited to, non-human primates, dogs, cats, rodents, horses, cows, pigs, mice, rats, hamsters, rabbits, and the like (e.g., which is to be the recipient of a particular treatment, or from whom cells are harvested). In some embodiments, the subject is a human.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration, or palliation of the disease condition, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein the term “cancer,” “cancerous tissue,” or “tumor” refers to an abnormal mass of tissue in which the growth of the mass surpasses and is not coordinated with the growth of normal tissue. In the case of hematological cancers, this includes a volume of blood or other bodily fluid containing cancerous cells. A cancer or tumor can be defined as “benign” or “malignant” depending on the following characteristics: degree of cellular differentiation including morphology and functionality, rate of growth, local invasion, and metastasis. A “benign” tumor can be well differentiated, have characteristically slower growth than a malignant tumor and remain localized to the site of origin. In addition, in some cases a benign tumor does not have the capacity to infiltrate, invade, or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites. Accordingly, a cancer cell is a cell found within the abnormal mass of tissue whose growth is not coordinated with the growth of normal tissue. Accordingly, a “tumor sample,” “tumor specimen,” or “somatic biopsy” refers to a biological sample obtained or derived from a tumor of a subject, as described herein.

In some embodiments, a tumor specimen or tumor sample is obtained from a site of biopsy in a subject, including but not limited to: lip, base of tongue, tongue (excluding base of tongue), gum, floor of mouth, & other mouth, salivary gland, oropharynx, nasopharynx (excluding posterior wall), posterior wall of nasopharynx, hypopharynx, pharynx, esophagus, stomach, small intestine, large intestine, (excluding appendix), appendix, rectum, anal canal & anus, liver, intrahepatic bile ducts, gallbladder & extrahepatic bile ducts, pancreas, unspecified digestive organs, nasal cavity (including nasal cartilage), middle ear, sinuses, accessory sinus, nose, larynx, trachea, lung & bronchus, thymus, heart, mediastinum, pleura, respiratory, bones & joints (excluding skull and face, mandible), bones of skull and face, mandible, blood, bone marrow, & hematopoietic sys, spleen, reticulo-endothelial, skin, peripheral nerves, retroperitoneum & peritoneum, connective & soft tissue, breast, vagina & labia, vulva, cervix uteri, corpus uteri, uterus, ovary, fallopian tube, other female genital (excluding fallopian tube), placenta, penis, prostate gland, testis, epididymis, spermatic cord, male genital, scrotum, kidney, renal pelvis, ureter, urinary bladder, other urinary organs, orbit & lacrimal gland, (excluding retina, eye, nose), retina, eyeball, eye, nose, meninges (e.g., cerebral and spinal), brain, & cranial nerves, & spinal cord, (excluding ventricle, cerebellum), ventricle, cerebellum, other nervous system, thyroid gland, adrenal glands, parathyroid gland, pituitary gland, craniopharyngeal duct, pineal gland, other endocrine glands, ill-defined, lymph nodes, and unknown.

In some embodiments, the cancer refers to Acute Lymphoblastic Leukemia (ALL), Acute Myeloid Leukemia (AML), Adolescents, Cancer in, Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Childhood (Brain Cancer), Atypical Teratoid/Rhabdoid Tumor, Childhood, Central Nervous System (Brain Cancer), Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors (Lung Cancer), Burkitt Lymphoma, Carcinoid Tumor (Gastrointestinal), Carcinoma of Unknown Primary, Cardiac (Heart) Tumors, Childhood, Central Nervous System, Atypical Teratoid/Rhabdoid Tumor, Childhood (Brain Cancer), Medulloblastoma and Other CNS Embryonal Tumors, Childhood (Brain Cancer), Germ Cell Tumor, Childhood (Brain Cancer), Primary CNS Lymphoma, Cervical Cancer, Childhood Cancers, Cancers of Childhood, Unusual, Cholangiocarcinoma, Chordoma, Childhood (Bone Cancer), Chronic Lymphocytic Leukemia (CLL), Chronic Myelogenous Leukemia (CML), Chronic Myeloproliferative Neoplasms, Colorectal Cancer, Craniopharyngioma, Childhood (Brain Cancer), Cutaneous T-Cell Lymphoma, Ductal Carcinoma In Situ (DCIS), Childhood (Brain Cancer), Endometrial Cancer (Uterine Cancer), Ependymoma, Childhood (Brain Cancer), Esophageal Cancer, Esthesioneuroblastoma (Head and Neck Cancer), Ewing Sarcoma (Bone Cancer), Extracranial Germ Cell Tumor, Childhood, Extragonadal Germ Cell Tumor, Eye Cancer, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST) (Soft Tissue Sarcoma), Germ Cell Tumors, Childhood Central Nervous System Germ Cell Tumors (Brain Cancer), Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Childhood, Hepatocellular (Liver) Cancer, Histiocytosis, Langerhans Cell, Hodgkin Lymphoma, Hypopharyngeal Cancer (Head and Neck Cancer), Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Leukemia, Lip and Oral Cavity Cancer (Head and Neck Cancer), Liver Cancer, Lung Cancer (Non-Small Cell, Small Cell, Pleuropulmonary Blastoma, and Tracheobronchial Tumor), Lymphoma, Male Breast Cancer, Malignant Fibrous Histiocytoma of Bone and Osteosarcoma, Melanoma, Melanoma, Intraocular (Eye), Merkel Cell Carcinoma (Skin Cancer), Mesothelioma, Malignant, Metastatic Cancer, Metastatic Squamous Neck Cancer with Occult Primary (Head and Neck Cancer), Midline Tract Carcinoma With NUT Gene Changes, Mouth Cancer (Head and Neck Cancer), Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, Chronic (CML), Myeloid Leukemia, Acute (AML), Myeloproliferative Neoplasms, Chronic, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer, Lip and Oral Cavity Cancer and Oropharyngeal Cancer (Head and Neck Cancer), Osteosarcoma and Malignant Fibrous Histiocytoma of Bone, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Pituitary Tumor, Plasma Cell Neoplasm/Multiple Myeloma, Pleuropulmonary Blastoma (Lung Cancer), Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Childhood (Soft Tissue Sarcoma), Salivary Gland Cancer (Head and Neck Cancer), Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Soft Tissue Sarcoma, Uterine Sarcoma, Sézary Syndrome (Lymphoma), Skin Cancer, Small Cell Lung Cancer, Small Intestine Cancer, Soft Tissue Sarcoma, Squamous Cell Carcinoma of the Skin, Squamous Neck Cancer with Occult Primary, Metastatic (Head and Neck Cancer), Stomach (Gastric) Cancer, T-Cell Lymphoma, Lymphoma (Mycosis Fungoides and Sezary Syndrome), Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Tracheobronchial Tumors (Lung Cancer), Transitional Cell Cancer of the Renal Pelvis and Ureter (Kidney (Renal Cell) Cancer), Ureter and Renal Pelvis, Transitional Cell Cancer (Kidney (Renal Cell) Cancer, Urethral Cancer, Uterine Cancer, Endometrial, Uterine Sarcoma, Vaginal Cancer, Vascular Tumors (Soft Tissue Sarcoma), and/or Vulvar Cancer.

In some embodiments, a cancer can be categorized to a cohort class. Exemplary cohort classes may include Blood Cancer, Bone Cancer, Brain Cancer, Bladder Cancer, Breast Cancer, Colon and Rectal Cancer, Endometrial Cancer, Kidney Cancer, Leukemia, Liver Cancer, Lung Cancer, Melanoma, Non-Hodgkin Lymphoma, Pancreatic Cancer, Prostate Cancer, Thyroid Cancer, or other tissue/organ-based classifications.

For the avoidance of doubt, it is intended herein that particular features (for example integers, characteristics, values, uses, diseases, formulae, compounds, or groups) described in conjunction with a particular aspect, embodiment or example of the disclosure are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The disclosure is not restricted to any details of any disclosed embodiments. The disclosure extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

Furthermore, the transitional terms “comprising,” “consisting essentially of” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step, or material. The term “consisting of” excludes any element, step, or material other than those specified in the claim and, in the latter instance, impurities ordinary associated with the specified material(s). The term “consisting essentially of” limits the scope of a claim to the specified elements, steps, or material(s) and those that do not materially affect the basic and novel characteristic(s) of the claimed invention. All embodiments of the invention can, in the alternative, be more specifically defined by any of the transitional terms “comprising,” “consisting essentially of,” and “consisting of.”

### Exemplary System Embodiments

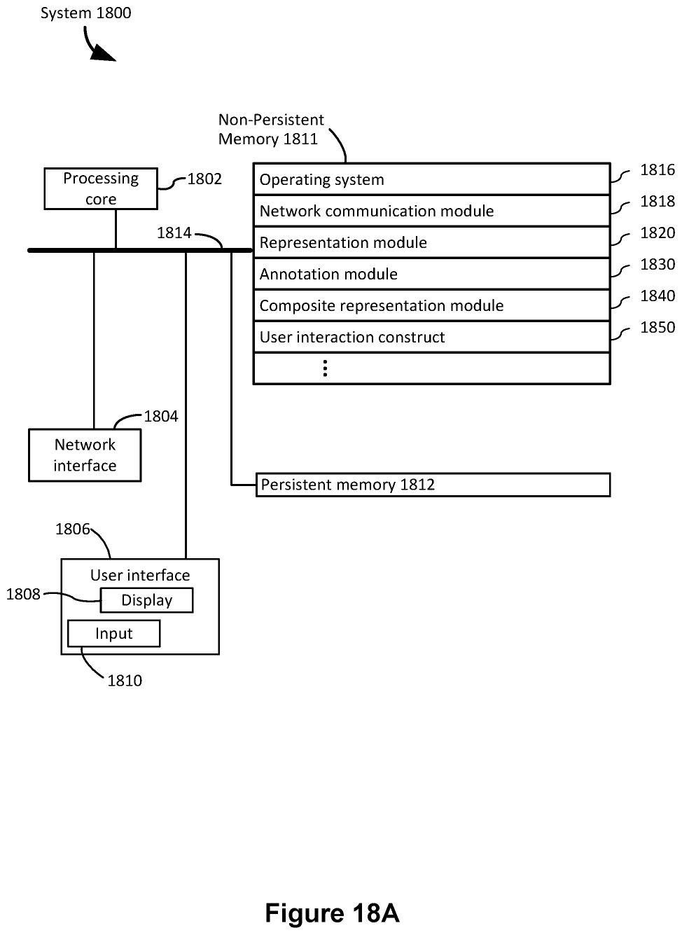

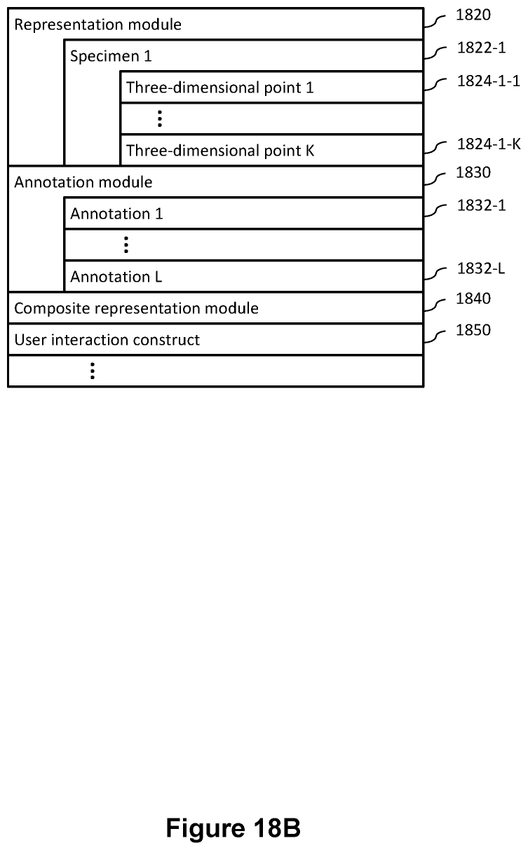

Details of an exemplary system are now described in conjunction with FIGS. 18A-B. FIGS. 18A-B are a block diagram illustrating system 1800 for localizing close or positive margins in a tumor specimen from a subject, in accordance with some implementations. System 1800 in some implementations includes one or more processing units CPU(s) 1802 (also referred to as processors or processing core), one or more network interfaces 1804, user interface 1806, display 1808, input 1810, non-persistent memory 1811, persistent memory 1812, and one or more communication buses 1814 for interconnecting these components. One or more communication buses 1814 optionally include circuitry (sometimes called a chipset) that interconnects and controls communications between system components. Non-persistent memory 1811 typically includes high-speed random access memory, such as DRAM, SRAM, DDR RAM, ROM, EEPROM, flash memory, whereas persistent memory 1812 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid-state storage devices. Persistent memory 1812 optionally includes one or more storage devices remotely located from the CPU(s) 1802. Persistent memory 1812, and the non-volatile memory device(s) within non-persistent memory 1812, comprise non-transitory computer-readable storage medium. In some implementations, non-persistent memory 1811 or alternatively non-transitory computer-readable storage medium stores the following programs, modules and data structures, or a subset thereof, sometimes in conjunction with persistent memory 1812:
instructions, programs, data, or information associated with an optional operating system 1816, which includes procedures for handling various basic system services and for performing hardware dependent tasks;
instructions, programs, data, or information associated with an optional network communication module 1818 for connecting the system 1800 with other devices, or a communication network;
instructions, programs, data, or information associated with representation module 1820 optionally including:
   a first three-dimensional representation 1822 (e.g., 1822-1) of the tumor specimen, where the three-dimensional representation comprises more than 1000 three-dimensional points 1824 (e.g., 1824-1-1, . . . 1824-1-K) that define a boundary of the tumor specimen;
instructions, programs, data, or information associated with annotation module 1830 optionally including:
   a first plurality of annotations 1832 (e.g., 1832-1, . . . 1832-L) to the first three-dimensional representation of the tumor specimen;
instructions, programs, data, or information associated with composite representation module 1840 optionally including a composite three-dimensional representation of the tumor specimen formed by modifying the first three-dimensional representation 1822 of the tumor specimen to include the first plurality of annotations 1832; and
an optional user interaction construct 1850 for receiving user input.

In some implementations, one or more of the above-identified elements are stored in one or more of the previously mentioned memory devices and correspond to a set of instructions for performing a function described above. The above-identified modules, data, or programs (e.g., sets of instructions) may not be implemented as separate software programs, procedures, datasets, or modules, and thus various subsets of these modules and data may be combined or otherwise re-arranged in various implementations. In some implementations, the non-persistent memory 1811 optionally stores a subset of the modules and data structures identified above. Furthermore, in some embodiments, the memory stores additional modules and data structures not described above. In some embodiments, one or more of the above-identified elements is stored in a computer system, other than that of system 1800, that is addressable by system 1800 so that system 1800 may retrieve all or a portion of such data.

Although FIGS. 18A-B collectively depict a “system 1800,” the figure is intended more as functional description of the various features which may be present in computer systems than as a structural schematic of the implementations described herein. In practice, items shown separately could be combined and some items can be separated. Moreover, although FIGS. 18A-B collectively depict certain data and modules in non-persistent memory 1811, some or all of these data and modules may be in persistent memory 1812.

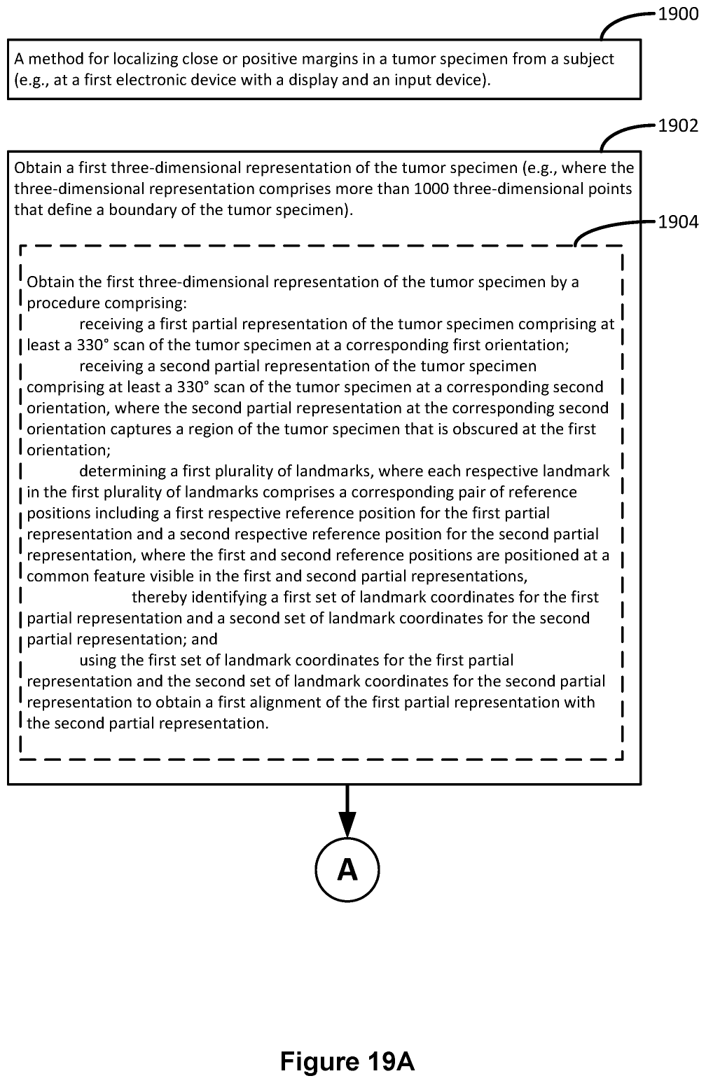

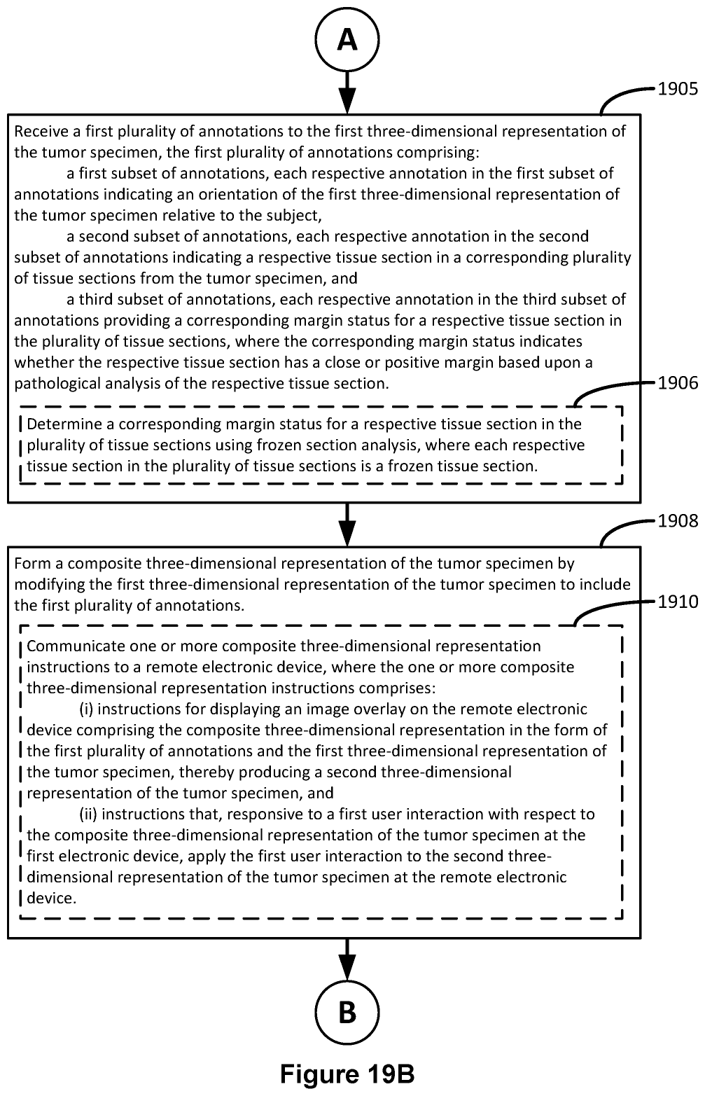

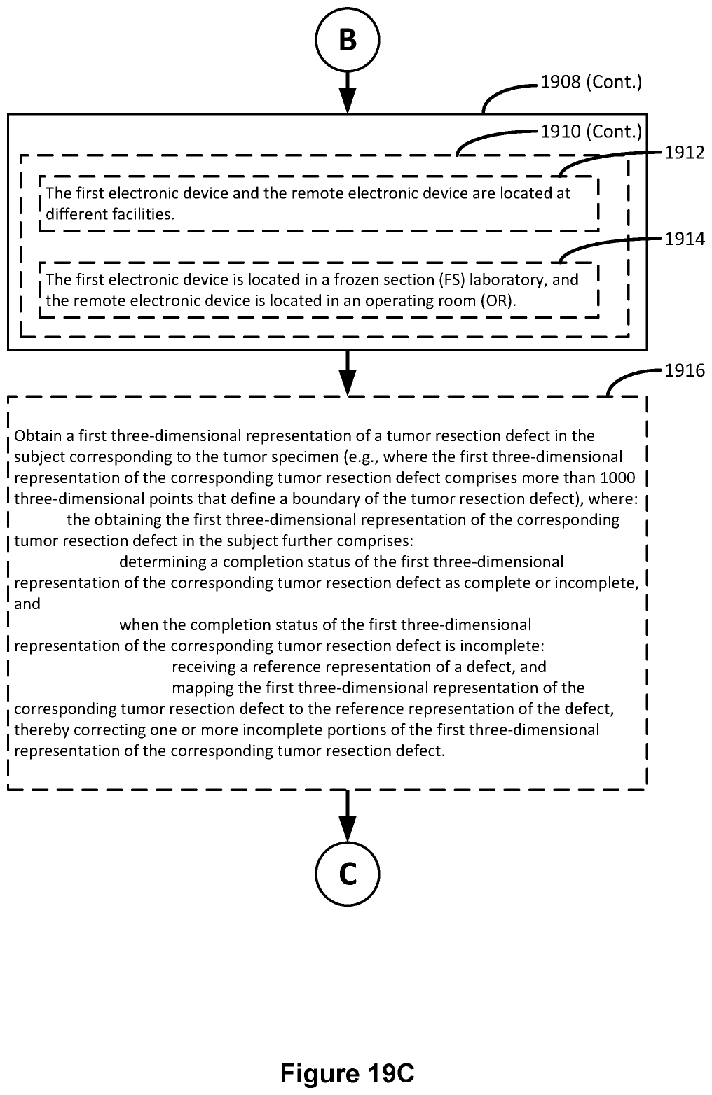

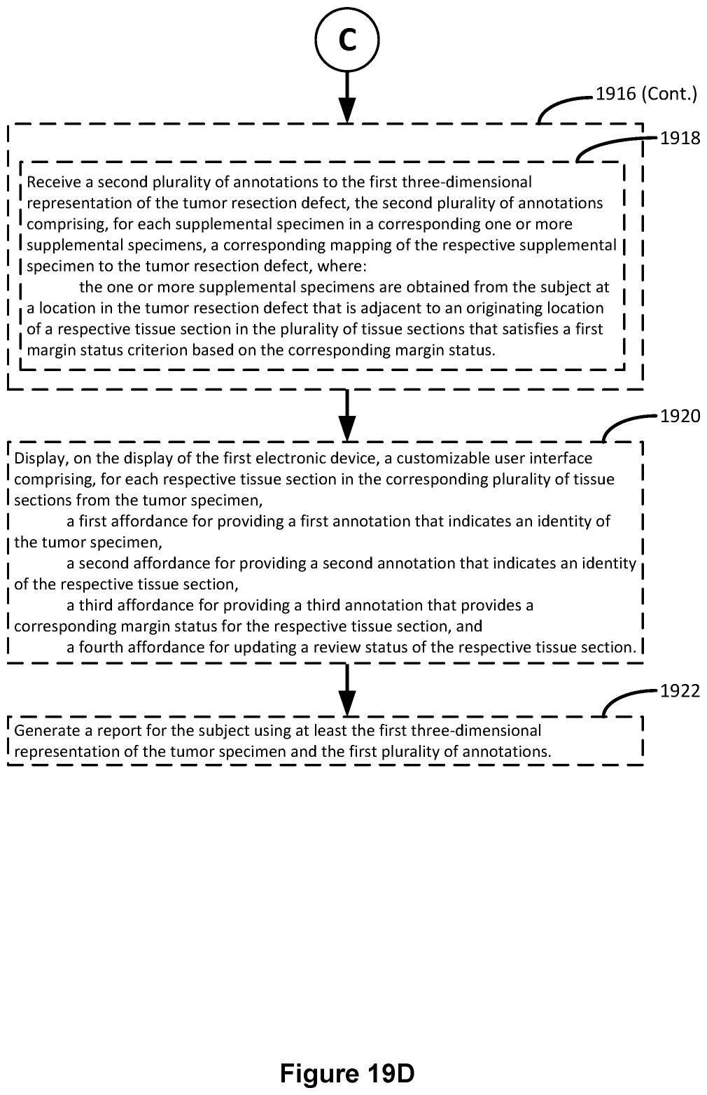

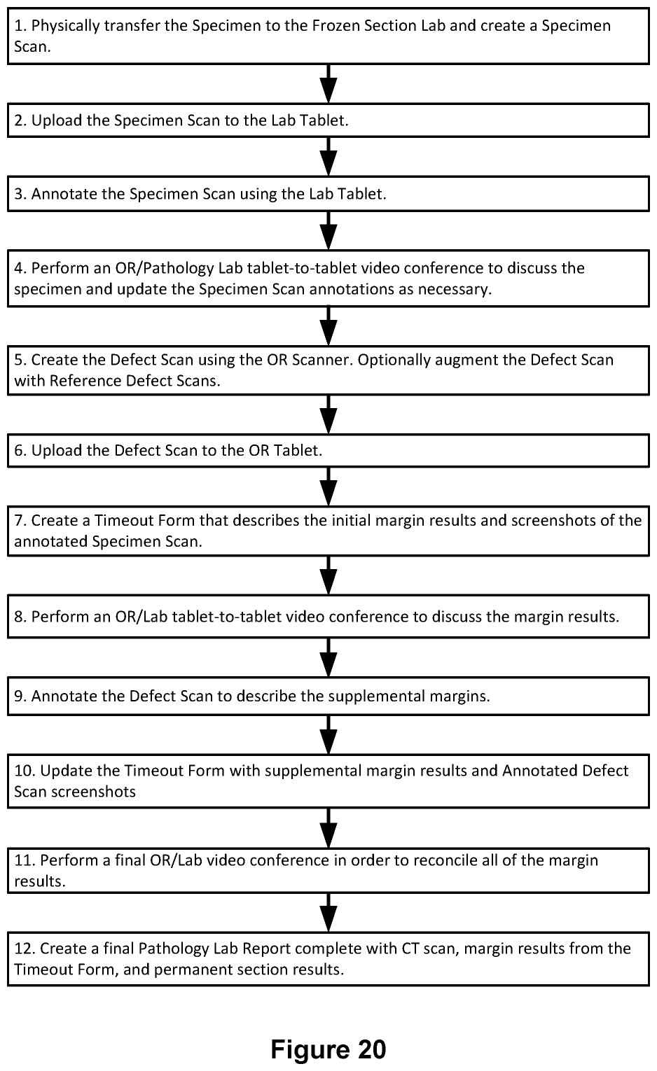

While a system in accordance with the present disclosure has been disclosed with reference to FIGS. 18A-B, methods in accordance with the present disclosure are now detailed with reference to FIGS. 19A-D and FIG. 20.

Example Embodiments for Localizing Close or Positive Margins in a Tumor Specimen from a Subject.

Accordingly, one aspect of the present disclosure provides a system 1800 comprising, at a first electronic device, a display, an input device, one or more processors, memory, and one or more programs, where the one or more programs are stored in the memory and configured to be executed by the one or more processors, the one or more programs including instructions for a method for localizing close or positive margins in a tumor specimen from a subject. Referring to FIGS. 19A-D, the one or more programs include instructions for a method 1900 for localizing close or positive margins in a tumor specimen from a subject.

In some embodiments, the method 1900 is performed at the first electronic device.

### Tumor Specimen Scan.

In some embodiments, the tumor specimen is from a cancer selected from the group consisting of adrenal cancer, biliary tract cancer, bladder cancer, bone/bone marrow cancer, brain cancer, breast cancer, cervical cancer, colorectal cancer, cancer of the esophagus, gastric cancer, head/neck cancer, hepatobiliary cancer, kidney cancer, liver cancer, lung cancer, ovarian cancer, pancreatic cancer, pelvis cancer, pleura cancer, prostate cancer, renal cancer, skin cancer, stomach cancer, testis cancer, thymus cancer, thyroid cancer, uterine cancer, lymphoma, melanoma, multiple myeloma, and leukemia.

Referring to Block 1902, in some embodiments, the method includes obtaining a first three-dimensional representation of the tumor specimen (e.g., where the three-dimensional representation comprises more than 1000 three-dimensional points that define a boundary of the tumor specimen).

In some embodiments, a respective three-dimensional point is any entity that defines all or a portion of a three-dimensional image, scan, and/or representation, such as a pixel and/or a voxel. In some embodiments, each respective three-dimensional point has a corresponding three-dimensional point value (e.g., a grayscale or RGB value).

In some embodiments, the three-dimensional representation of the tumor specimen comprises a plurality of three-dimensional points. In some embodiments, the plurality of three-dimensional points comprises at least 2000, at least 5000, at least 10,000, at least 20,000, at least 30,000, at least 40,000, at least 50,000, at least 100,000, at least 250,000, at least 500,000, at least 1 million, at least 1.5 million, at least 2 million, at least 3 million, or at least 5 million points. In some embodiments, the plurality of three-dimensional points comprises no more than 10 million, no more than 5 million, no more than 1 million, no more than 500,000, no more than 100,000, no more than 50,000, or no more than 10,000 points. In some embodiments, the plurality of three-dimensional points consists of from 5000 to 50,000, from 10,000 to 500,000, from 50,000 to 1 million, or from 1 million to 10 million points. In some embodiments, the plurality of three-dimensional points falls within another range starting no lower than 2000 points and ending no higher than 10 million points.

Referring to Block 1904, in some embodiments, the method further includes obtaining the first three-dimensional representation of the tumor specimen by a procedure (e.g., a scanning procedure). In some embodiments, the procedure includes receiving a first partial representation of the tumor specimen comprising at least a 330° scan of the tumor specimen at a corresponding first orientation and receiving a second partial representation of the tumor specimen comprising at least a 330° scan of the tumor specimen at a corresponding second orientation, where the second partial representation at the corresponding second orientation captures a region of the tumor specimen that is obscured at the first orientation. In some embodiments, a first plurality of landmarks is determined. Each respective landmark in the first plurality of landmarks comprises a corresponding pair of reference positions including a first respective reference position for the first partial representation and a second respective reference position for the second partial representation, where the first and second reference positions are positioned at a common feature visible in the first and second partial representations. Thus, a first set of landmark coordinates for the first partial representation and a second set of landmark coordinates for the second partial representation are identified. The procedure further includes using the first set of landmark coordinates for the first partial representation and the second set of landmark coordinates for the second partial representation to obtain a first alignment of the first partial representation with the second partial representation.

In some embodiments, the procedure is a scanning procedure. In some embodiments, the scanning procedure includes receiving one or more scans of the tumor specimen, where each respective scan is of the tumor specimen at a corresponding orientation in one or more orientations. In some embodiments, each respective orientation in the one or more orientations is created by manually repositioning the tumor specimen (e.g., by flipping, rotating, and/or translating). In some embodiments, the one or more scans of the tumor specimen comprises at least 1, at least 2, at least 3, at least 4, at least 5, at least 6, at least 7, at least 8, at least 9, or at least 10 scans. In some embodiments, the one or more scans of the tumor specimen comprises no more than 20, no more than 10, no more than 8, no more than 5, no more than 3, no more than 2, or no more than 1 scan. In some embodiments, the one or more scans of the tumor specimen consists of from 1 to 3, from 2 to 5, from 3 to 10, or from 8 to 20 scans. In some embodiments, the one or more scans of the tumor specimen falls within another range starting no lower than 1 scan and ending no higher than 20 scans. In some embodiments, the procedure includes receiving a single scan of the tumor specimen.

In some embodiments, a respective scan in the one or more scans is a full or partial representation of the tumor specimen. In some embodiments, a respective scan comprises at least a 120° scan, at least a 150° scan, at least a 180° scan, at least a 210° scan, at least a 240° scan, at least a 270° scan, at least a 300° scan, at least a 330° scan, at least a 340° scan, at least a 350° scan, or at least a 355° scan of the tumor specimen. In some embodiments, a respective scan comprises no more than a 360° scan, no more than a 330° scan, no more than a 300° scan, no more than a 270° scan, no more than a 240° scan, no more than a 210° scan, no more than a 180° scan, or no more than a 150° scan. In some embodiments, a respective scan consists of from 120° to 180°, from 180° to 240°, from 240° to 300°, or from 300° to 360°. In some embodiments, a respective scan falls within another range starting no lower than 120° and ending no higher than 360°.

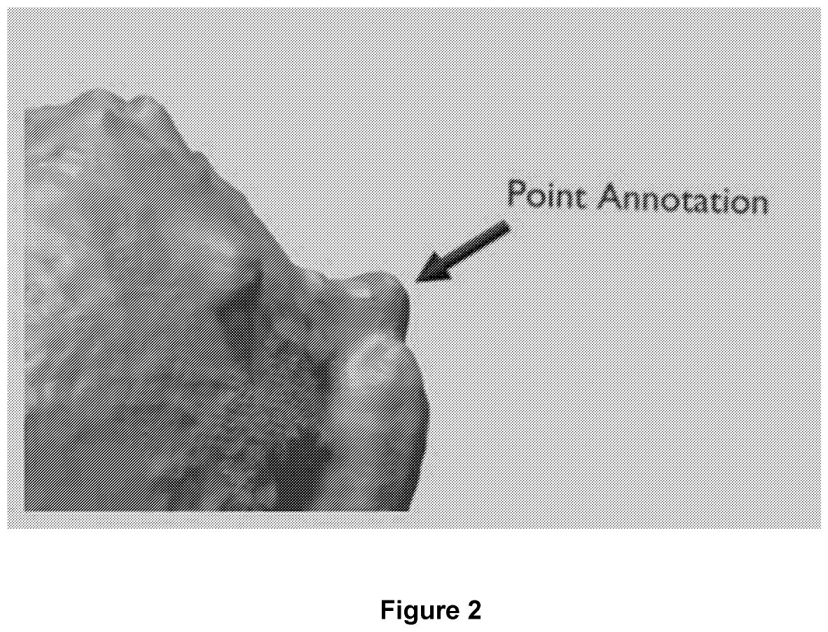

In some embodiments, the first plurality of landmarks comprises at least 2, at least 5, at least 10, at least 20, or at least 50 landmarks. In some embodiments, the first plurality of landmarks comprises no more than 100, no more than 50, no more than 20, no more than 10, or no more than 5 landmarks. In some embodiments, the first plurality of landmarks consists of from 2 to 10, from 5 to 20, from 15 to 50, or from 20 to 100 landmarks. In some embodiments, the first plurality of landmarks falls within another range starting no lower than 2 landmarks and ending no higher than 100 landmarks. In some embodiments, a respective landmark in the first plurality of landmarks is a physical characteristic of the tumor specimen (e.g., a distinguishing feature in the tumor specimen). In some embodiments, a respective landmark in the first plurality of landmarks is a point annotation, such as illustrated in FIG. 2. In some embodiments, a respective landmark in the first plurality of landmarks is a fiducial marker that is manually added to the tumor specimen or adjacent to the tumor specimen, such as a pin, a paint marker, and/or a tag. In some embodiments, a respective landmark in the first plurality of landmarks is a colored pin.

In some embodiments, the one or more scans is a plurality of partial scans, and the procedure further includes combining the plurality of partial scans into the first three-dimensional representation of the tumor specimen. In some embodiments, the combining does not comprise the use of landmarks.

In some embodiments, a respective scan of the tumor specimen is obtained using a first three-dimensional scanner. Any suitable three-dimensional scanner that is commercially available and/or known in the art is contemplated for use in the present disclosure. In some embodiments, the first three-dimensional scanner is used to obtain one or more three-dimensional scans of the tumor specimen, and a second three-dimensional scanner is used to obtain one or more three-dimensional scans of a corresponding tumor resection defect, as detailed further elsewhere (see, e.g., the section entitled “Tumor resection defect scan,” below). In some embodiments, the first three-dimensional scanner is the same or different from the second three-dimensional scanner.

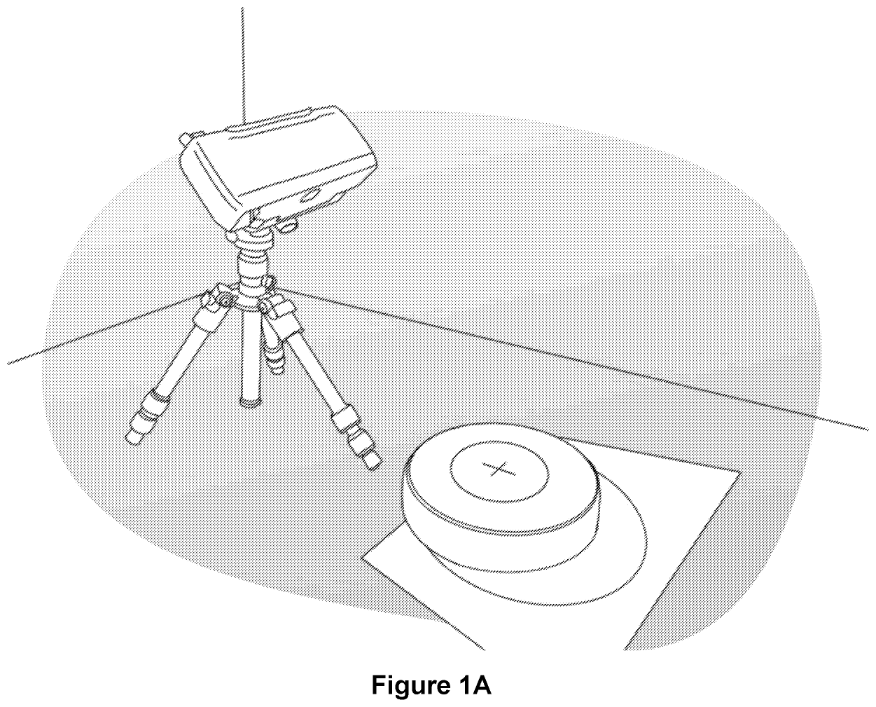

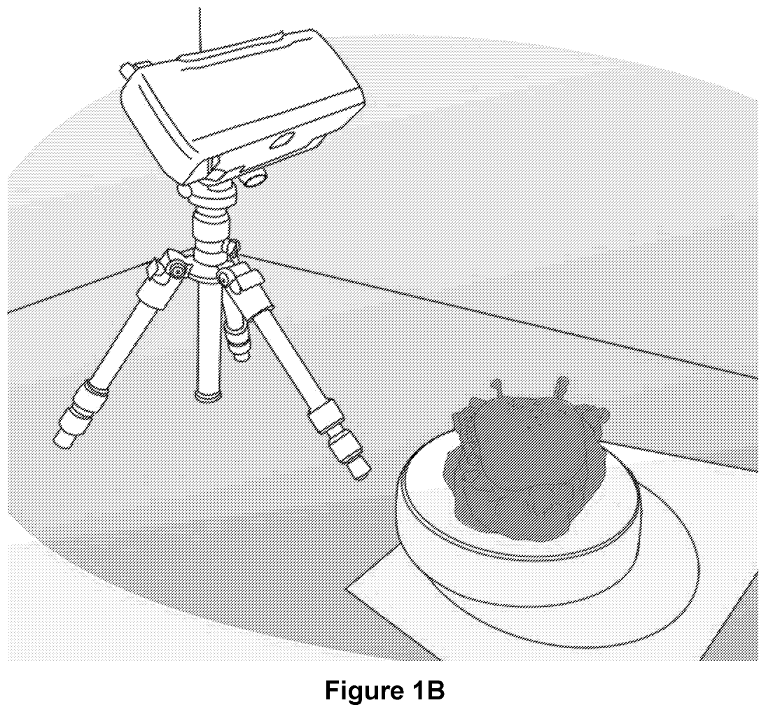

In some embodiments, as illustrated in FIGS. 1A-B, the first three-dimensional scanner is affixed to a solid support (e.g., a platform), and the tumor specimen is moved in space in order to obtain the one or more scans. In some embodiments, the first three-dimensional scanner is freely movable in space and the tumor specimen is stationary. In some embodiments, both the first three-dimensional scanner and the tumor specimen are freely movable or stationary. In some embodiments, the first three-dimensional scanner is connected (e.g., via a network communication module 1818 and/or communication buses 1814) to the first electronic device.

As an example, in some embodiments, the first three-dimensional scanner is a specimen scanner. In some implementations, the specimen scanner is located in a first facility (e.g., a frozen section room). In some embodiments, the specimen scanner is fixed in space (e.g., affixed to a solid support), has a turntable for the specimen, and is capable of scanning the entire surface of the specimen into one or more scans. In some embodiments, the specimen scanner is capable of millimeter resolution, texture capture, and/or color capture. In some implementations where more than one scan is obtained, the method further includes combining the multiple scans together into a single representation in order to create a three-dimensional computer representation of the specimen, optionally referred to herein as a “3D Specimen Scan.”

In some embodiments, the specimen scanner is attached (e.g., via cable, communication bus 1814, and/or network communication module 1818) to a PC and/or a tablet with an application that controls the scanner. In some embodiments, the first electronic device comprises a computer (e.g., a PC) and/or a tablet. In some embodiments, the first electronic device is capable of direct manipulation (e.g., using a mouse, a stylus, and/or a touch screen interface), comprises software that supports uploading and storing multiple three-dimensional scans, comprises software that supports annotating a 3D scan using direct manipulation; and/or comprises video conferencing software that can integrate with the annotation software. Any suitable PC or tablet that is commercially available and/or known in the art is contemplated for use in the present disclosure.

In some embodiments, tumor specimen is obtained in a first facility.

In some embodiments, the first facility is an operating room (OR), and the tumor specimen is obtained via surgery.

In some embodiments, the method further includes obtaining one or more supplemental specimens from the subject (e.g., in the first facility) (see, for example, the section entitled “Tumor resection defect scan,” below).

In some embodiments, the method further includes scanning and/or annotating a tumor resection defect corresponding to the tumor specimen (e.g., in the first facility) (see, for example, the section entitled “Tumor resection defect scan,” below). For example, in some embodiments, the defect is scanned in the operating room (OR) after the tumor specimen is removed from the subject, and the defect scan is annotated, in the operating room (OR), at each location from which a supplemental specimen is obtained.

In some embodiments, the tumor specimen is transported to a second facility. In some embodiments, the second facility is the same or different from the first facility. In some embodiments, the second facility is a pathology laboratory. In some embodiments, the second facility is a frozen section (FS) laboratory.

In some embodiments, the one or more supplemental specimens are transported to the second facility.

In some embodiments, the obtaining the first three-dimensional representation of the tumor specimen is performed at the second facility. For example, in some embodiments, the tumor specimen and/or the one or more supplemental specimens are scanned at the second facility after relocation or transportation from the first facility.

In some embodiments, the method further includes obtaining a corresponding plurality of tissue sections from the tumor specimen (e.g., at the second facility).

In some embodiments, the plurality of tissue sections comprises at least 2, at least 3, at least 4, at least 5, at least 6, at least 7, at least 8, at least 9, or at least 10 tissue sections. In some embodiments, the plurality of tissue sections comprises no more than 20, no more than 10, no more than 8, no more than 5, no more than 3 tissue sections. In some embodiments, the plurality of tissue sections consists of from 2 to 5, from 3 to 8, from 5 to 10, or from 8 to 20 tissue sections. In some embodiments, the plurality of tissue sections falls within another range starting no lower than 2 tissue sections and ending no higher than 20 tissue sections.

In some embodiments, the method further includes obtaining a first plurality of annotations for the tumor specimen and/or the first three-dimensional representation thereof (e.g., at the second facility) (see, for example, the section entitled “Annotations,” below).

In some embodiments, the method further includes obtaining a corresponding margin status for a respective tissue section in the plurality of tissue sections (e.g., performing margin analysis) at the second facility. In some embodiments, the margin analysis is performed by processing frozen sections from the specimens. Alternatively or additionally, in some embodiments, the corresponding margin status for a respective tissue section in the plurality of tissue sections is performed using permanent section analysis. Typically, frozen section analysis is performed more rapidly (e.g., between about 10 minutes and 1 hour) than permanent section analysis (e.g., between about 1 day and 3 days), and can advantageously be used intraoperatively for feedback and supplemental margin harvesting.

An example workflow for the use of three-dimensional representations of a tumor specimen and a tumor resection defect to enhance communication and documentation of tissue sections from the tumor specimen is illustrated in FIG. 20 and described in further detail below, in accordance with an embodiment of the present disclosure.

Referring to Step 1, the example workflow includes physically transferring a tumor specimen to a frozen section lab and creating a specimen scan. Generally, commercial scanners are either packaged with a PC, or include specifications for a PC that is to be connected to the scanner. The specifications typically describe a powerful processor with a significant amount of RAM, and usually a graphics card. In the example workflow, the frozen section lab is set up prior to scanning. Three main components are set up and configured: the specimen scanner, the PC, and the physical specimen. A turntable is used as a fixture whose rotation supports scanning the entire surface of the specimen using two scans (e.g., a top scan and a bottom scan). In some embodiments, other fixtures and scanners are used, such as a fixture that positions the specimen in space so that a single scan can be used to reach all points of the specimen surface. An example protocol that utilizes a turntable is as follows: (1) the 3D specimen scanner is rigidly mounted on a tripod, and the tripod is positioned so that the scanner can point down to the top of the turntable; (2) the positioning of the turntable is adjusted so that the scanner has an unobstructed view and is pointed down at a steep angle to the center of the turntable; (3) the turntable is further covered with a protective piece of plastic that does not interfere with turntable rotation; (4) using gloves, the specimen is centered on the turntable such that it lies flat and is positioned so that it can be flipped over; and (5) colored pins (e.g., landmarks) of at least two different colors are inserted deeply into 2 to 4 points on the specimen to aid with scan alignment later. Advantageously, the use of at least two different colors allows for distinguishing between points (e.g., pairs of reference positions). Further, in the example protocol: (6) the lab room lights are adjusted so as not to interfere with the light source from the scanner; and (7) after confirming that the PC is connected to the network, the scan is initialized using a companion application that runs on the PC. Specimen scanning with a turntable typically requires two separate rounds of scanning, one for the top and one for the bottom of the specimen. Typically, additional processing is performed in the companion PC application in order to ensure that the scan is valid and complete. This processing is usually an algorithm that is part of the companion PC application. Accordingly, in the example protocol: (8) using gloves, the specimen is inverted (e.g., flipped) so that the bottom is exposed; (9) the scanning step is repeated in order to scan the bottom half of the specimen; and (10) the companion PC application is then used to align the top and bottom scans and combine them into a single three-dimensional representation.

Referring to Step 2 in FIG. 20, the example workflow further includes uploading the specimen scan to a lab tablet. For instance, once the scan is complete and validated, it is transferred to the lab tablet. This is facilitated using a local computer network with the PC, the tablet, and an ability to store files into and retrieve them from a network folder. There are three steps to this process: (1) on the lab PC, the underlying computer representation of the specimen scan is transformed to one that the software on the lab tablet is capable of processing; (2) on the lab PC, the resulting scan is saved to a network folder that is accessible from the lab tablet; and (3) on the lab tablet, the specimen scan is uploaded from the network folder.

### Annotations.

Referring to Block 1905, in some embodiments, the method 1900 further includes receiving a first plurality of annotations to the first three-dimensional representation of the tumor specimen. In some embodiments, the first plurality of annotations comprises a first subset of annotations, each respective annotation in the first subset of annotations indicating an orientation of the first three-dimensional representation of the tumor specimen relative to the subject. In some embodiments, the first plurality of annotations further includes a second subset of annotations, each respective annotation in the second subset of annotations indicating a respective tissue section in a corresponding plurality of tissue sections from the tumor specimen. In some embodiments, the first plurality of annotations further includes a third subset of annotations, each respective annotation in the third subset of annotations providing a corresponding margin status for a respective tissue section in the plurality of tissue sections, where the corresponding margin status indicates whether the respective tissue section has a close or positive margin based upon a pathological analysis of the respective tissue section.

In some embodiments, the first plurality of annotations comprises at least 2, at least 3, at least 4, at least 5, at least 6, at least 7, at least 8, at least 9, at least 10, at least 20, or at least 50 annotations. In some embodiments, the first plurality of annotations comprises no more than 100, no more than 30, no more than 20, no more than 10, no more than 8, no more than 5, no more than 3 annotations. In some embodiments, the first plurality of annotations consists of from 2 to 10, from 5 to 20, from 10 to 30, or from 25 to 100 annotations. In some embodiments, the first plurality of annotations falls within another range starting no lower than 2 annotations and ending no higher than 100 annotations.

In some embodiments, one or more annotations are added manually (e.g., using a mouse or a stylus on a device, such as a computer or a tablet). In some embodiments, one or more annotations are performed using a touchscreen device. In some embodiments, one or more annotations are added to the 3D representation based on an indication of a region of the 3D representation with which a respective annotation is associated (e.g., a text label assigned to a coordinate position in the 3D representation). In some embodiments, one or more annotations are added using automation.

In some embodiments, each respective annotation in the first subset of annotations indicates an orientation of the first three-dimensional representation of the tumor specimen relative to the subject, relative to a respective region or portion of the subject's body (e.g., a tumor resection defect), and/or relative to any suitable reference point associated with the obtaining of the tumor specimen (e.g., a predetermined reference point in the operating room). In some embodiments, each respective annotation in the first subset of annotations is selected from the group consisting of: anterior, posterior, lateral, medial, right, left, superior, and inferior.

In some embodiments, a respective annotation in the second subset of annotations that indicates a respective tissue section in a corresponding plurality of tissue sections from the tumor specimen comprises a corresponding label for the respective tissue section. In some embodiments, the label is a number (e.g., 1, 2, 3, 4, etc.), an alphanumeric string, an asterisk, an arrow, a geometric shape, and/or any other suitable symbol or identifier. In some embodiments, a respective annotation in the second subset of annotations comprises a delineator between a first tissue section and a second tissue section (e.g., a line showing where sections are cut within the tumor specimen). In some embodiments, a respective annotation in the second subset of annotations identifies a position in the tumor specimen, or a three-dimensional representation thereof, from which the respective tissue section was obtained.

Referring to Block 1906, in some embodiments, the method further includes determining a corresponding margin status for a respective tissue section in the plurality of tissue sections using frozen section analysis, where each respective tissue section in the plurality of tissue sections is a frozen tissue section. In some embodiments, each respective tissue section in the plurality of tissue sections is subjected to frozen tissue analysis. Alternatively or additionally, in some embodiments, each respective tissue section in the plurality of tissue sections is subjected to permanent section analysis.

In some embodiments, a first portion of the tumor specimen comprises cancerous tissue, a second portion of the tumor specimen comprises normal tissue, and the corresponding margin status for a respective tissue section indicates that the respective tissue section has a close margin when a distance between a leading edge of the cancerous tissue and an outer surface of the tumor specimen satisfies a first distance criterion. In some embodiments, the distance between a leading edge of the cancerous tissue and an outer surface of the tumor specimen satisfies the first distance criterion when the distance is between a first threshold distance and a second threshold distance that is greater than the first threshold distance, where the first threshold distance is at least about 1 mm, at least about 2 mm, or at least about 3 mm, and the second threshold distance is no more than about 6 mm, no more than about 5 mm, or no more than about 4 mm.

Typically, a margin refers to an edge or border of a tumor specimen or a tissue section obtained therefrom, and margin analysis refers to a procedure where a margin is evaluated for the presence of cancerous tissue (e.g., the presence or absence of cancerous tissue at an edge or border of the tumor specimen). Generally, where cancerous tissue is determined to be at or close to an outer edge of the tumor specimen, the margin is determined to be close or positive (e.g., “at risk”), and where cancerous tissue is not found within a margin of the tumor specimen, the margin is determined to be negative or clean.

In some embodiments, the first threshold distance is at least about 1 mm, at least about 2 mm, at least about 3 mm, at least about 4 mm, or at least about 5 mm. In some embodiments, the first threshold distance is no more than 8 mm, no more than 5 mm, no more than 3 mm, or no more than 2 mm. In some embodiments, the first threshold distance is from 1 to 5 mm, from 2 to 7 mm, or from 3 to 8 mm. In some embodiments, the first threshold distance falls within another range starting no lower than 1 mm and ending no higher than 8 mm. In some embodiments, the second threshold distance is at least about 3 mm, at least about 4 mm, at least about 5 mm, at least about 6 mm, or at least about 7 mm. In some embodiments, the second threshold distance is no more than 10 mm, no more than 8 mm, no more than 5 mm, or no more than 4 mm. In some embodiments, the second threshold distance is from 3 to 7 mm, from 4 to 6 mm, or from 5 to 10 mm. In some embodiments, the second threshold distance falls within another range starting no lower than 3 mm and ending no higher than 10 mm.

In some embodiments, a respective tissue section for the tumor specimen is determined to have a “close” margin when the distance between a leading edge of the cancerous tissue and an outer surface of the tumor specimen is between 2 mm and 5 mm.

Alternatively or additionally, in some embodiments, a first portion of the tumor specimen comprises cancerous tissue, a second portion of the tumor specimen comprises normal tissue, and the corresponding margin status for a respective tissue section indicates that the respective tissue section has a positive margin when a distance between a leading edge of the cancerous tissue and an outer surface of the tumor specimen satisfies a second distance criterion. In some embodiments, the distance between a leading edge of the cancerous tissue and an outer surface of the tumor specimen satisfies the second distance criterion when the distance is less than a third threshold distance, where the third threshold distance is no more than about 3 mm, no more than about 2 mm, or no more than about 1 mm.

In some embodiments, the third threshold distance is at least about 1 mm, at least about 2 mm, at least about 3 mm, at least about 4 mm, or at least about 5 mm. In some embodiments, the third threshold distance is no more than 8 mm, no more than 5 mm, no more than 3 mm, or no more than 2 mm. In some embodiments, the third threshold distance is from 1 to 5 mm, from 2 to 7 mm, or from 3 to 8 mm. In some embodiments, the third threshold distance falls within another range starting no lower than 1 mm and ending no higher than 8 mm.

In some embodiments, a respective tissue section for the tumor specimen is determined to have a “positive” margin when the distance between a leading edge of the cancerous tissue and an outer surface of the tumor specimen is less than 2 mm.

In some embodiments, the corresponding margin status further indicates whether the respective tissue section has a negative margin based upon the pathological analysis of the respective tissue section. In some such embodiments, a first portion of the tumor specimen comprises cancerous tissue, a second portion of the tumor specimen comprises normal tissue, and the corresponding margin status for a respective tissue section indicates that the respective tissue section has a negative margin when a distance between a leading edge of the cancerous tissue and an outer surface of the tumor specimen satisfies a third distance criterion. In some embodiments, the distance between a leading edge of the cancerous tissue and an outer surface of the tumor specimen satisfies the third distance criterion when the distance is less than a fourth threshold distance, where the fourth threshold distance is at least about 4 mm, at least about 5 mm, or at least about 6 mm.

In some embodiments, the fourth threshold distance is at least about 3 mm, at least about 4 mm, at least about 5 mm, at least about 6 mm, or at least about 7 mm. In some embodiments, the fourth threshold distance is no more than 10 mm, no more than 8 mm, no more than 5 mm, or no more than 4 mm. In some embodiments, the fourth threshold distance is from 3 to 7 mm, from 4 to 6 mm, or from 5 to 10 mm. In some embodiments, the fourth threshold distance falls within another range starting no lower than 3 mm and ending no higher than 10 mm.

In some embodiments, a respective tissue section for the tumor specimen is determined to have a “negative” margin when the distance between a leading edge of the cancerous tissue and an outer surface of the tumor specimen is at least 5 mm.

In some embodiments, the first and the third threshold distance are the same or different. In some embodiments, the second and the fourth threshold distance are the same or different.

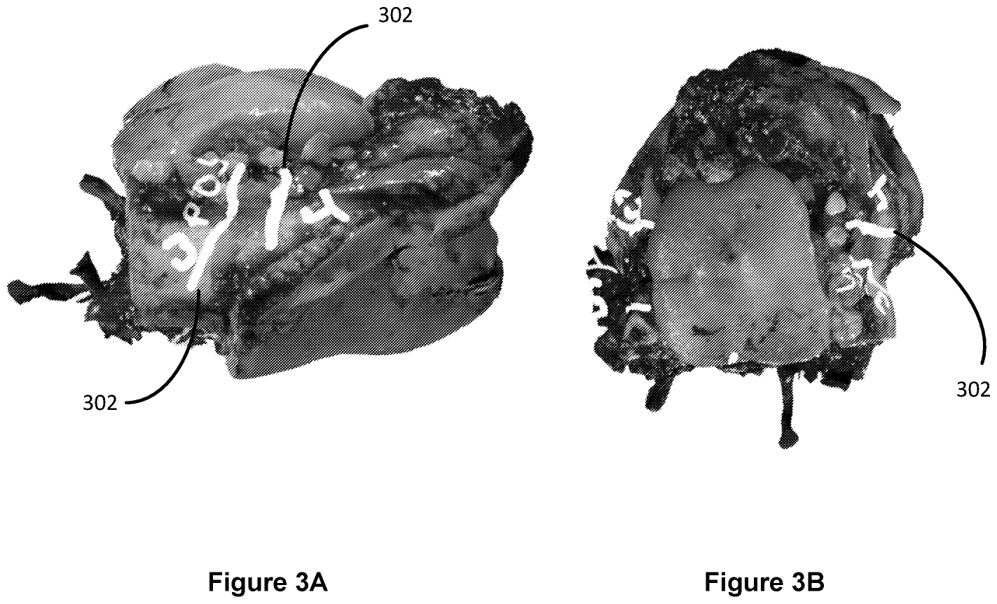

In some embodiments, a respective annotation in the first plurality of annotations is received in electronic form. In some embodiments, a respective annotation in the first plurality of annotation is received via direct manipulation to the first electronic device and/or to a remote electronic device (e.g., using a mouse, a stylus, and/or a touch screen interface). For example, in some implementations, the first plurality of annotations is obtained by marking up the first three-dimensional representation of the tumor specimen at a first electronic device in a first facility (e.g., a tablet at the FS laboratory) with, e.g., one or more labels indicating the orientation of the tumor specimen, one or more delineators indicating a location where a respective tissue section was cut, one or more labels indicating an identity of the respective tissue section, and/or one or more labels indicating a margin status of the respective tissue section. An example illustration of a three-dimensional representation of a tumor specimen comprising a first plurality of annotations is illustrated, for instance, in FIGS. 3A-B, 5C, 6D, 7, and 8B.

### Composite Representations.

Referring to Block 1908, in some embodiments, the method 1900 further includes forming a composite three-dimensional representation of the tumor specimen by modifying the first three-dimensional representation of the tumor specimen to include the first plurality of annotations.

In some embodiments, the method further includes, responsive to user input, turning the composite three-dimensional representation in three-dimensional space, where the receiving the first plurality of annotations to the first three-dimensional representation of the tumor specimen comprises performing a manual electronic annotation of the first three-dimensional representation, for each respective annotation in the first plurality of annotations, to produce the composite three-dimensional representation.

For example, in some implementations, one or more annotations in the first plurality of annotations are added by a procedure including (i) performing a user action to turn the three-dimensional representation displayed on the first electronic device and (ii) performing a user action to manually add the one or more annotations to the three-dimensional representation displayed on the first electronic device. In some embodiments the (i) turning and/or the (ii) adding includes the use of a mouse or a stylus to turn a 3D representation of the tumor specimen displayed on a device while marking where tissue sections were obtained from the tumor specimen, labeling the tissue sections with an identifier, and/or labeling the tissue sections with a margin status.

In some embodiments, the method further includes, responsive to a user interaction, performing an action on all or a portion of the composite three-dimensional representation selected from the group consisting of: changing a scale of the composite three-dimensional representation, panning the composite three-dimensional representation, rotating the composite three-dimensional representation, and highlighting a user selected portion of the composite three-dimensional representation. In this way, in some implementations, the first three-dimensional representation of the tumor specimen, the first plurality of annotations, and/or the composite three-dimensional representation thereof are interactive (e.g., can be generated or manipulated via user interaction).

### Communication of Three-Dimensional Representations.

In some embodiments, a respective three-dimensional representation (e.g., a scan), a corresponding plurality of annotations, and/or a composite three-dimensional representation thereof are communicated from a first electronic device (e.g., a PC or tablet located in a frozen section laboratory) to a second electronic device (e.g., a remote electronic device, such as a PC or tablet located in an operating room).

Referring to Block 1910, in some embodiments, the method further includes communicating one or more composite three-dimensional representation instructions to a remote electronic device where the one or more composite three-dimensional representation instructions comprises (i) instructions for displaying an image overlay on the remote electronic device comprising the composite three-dimensional representation in the form of the first plurality of annotations and the first three-dimensional representation of the tumor specimen, thereby producing a second three-dimensional representation of the tumor specimen, and (ii) instructions that, responsive to a first user interaction with respect to the composite three-dimensional representation of the tumor specimen at the first electronic device, apply the first user interaction to the second three-dimensional representation of the tumor specimen at the remote electronic device.

In some embodiments, the first user interaction is selected from the group consisting of changing a scale of the composite three-dimensional representation, panning the composite three-dimensional representation, rotating composite three-dimensional representation, and highlighting a user selected portion of the composite three-dimensional representation. In some embodiments, the first user interaction comprises adding one or more annotations to the first three-dimensional representation of the tumor specimen. In some embodiments, the first user interaction comprises selecting one or more portions of the composite three-dimensional representation. In some embodiments, the first user interaction comprises selecting an annotation that is overlaid onto the composite three-dimensional representation of the tumor specimen. In some implementations, the selection highlights the annotation on the composite three-dimensional representation of the tumor specimen (e.g., at the first electronic device and/or at the remote electronic device).

Referring to Block 1912, in some embodiments, the first electronic device and the remote electronic device are located at different facilities. In some embodiments, the first electronic device is located in a pathology laboratory, and the remote electronic device is located in an operating room (OR). Referring to Block 1914, in some embodiments, the first electronic device is located in a frozen section (FS) laboratory, and the remote electronic device is located in an operating room (OR). In this way, in some implementations, the first three-dimensional representation of the tumor specimen, the first plurality of annotations, and/or the composite three-dimensional representation thereof are interactively communicated (e.g., can be generated or manipulated via user interaction, which is transmitted in real-time to a remote device).

In some embodiments, the method further includes performing the first user interaction using a mouse or a stylus. In some embodiments, the method further includes formatting at least the first three-dimensional representation of the tumor specimen and/or the first plurality of annotations prior to the communicating.

In some embodiments, the communicating is bidirectional. In some embodiments, as described below, the method further includes performing, at the remote electronic device, a second user interaction with respect to the second three-dimensional representation of the tumor specimen at the remote electronic device. In some embodiments, the method further includes applying the second user interaction to the composite three-dimensional representation of the tumor specimen at the first electronic device (see, e.g., the section entitled “Additional embodiments.”). In other words, in some embodiments, the method includes displaying an interactive three-dimensional representation of the tumor specimen on each of a first and a second (e.g., remote) electronic device, such as a first PC or tablet and a second PC or tablet that are linked by a network communication module 1818. In some embodiments, the method allows for one or more user interactions at one or both of the first and the second electronic device, where a respective user interaction performed at one of the electronic devices is displayed concurrently on the other of the electronic devices. In this way, in some embodiments, the method facilitates interactive and, optionally, bidirectional communication. In some implementations, the second user interaction comprises any of the embodiments for the first user interaction disclosed elsewhere herein. Suitable embodiments for user interactions, include, but are not limited to, changing a scale of an image and/or scan, panning an image and/or scan, rotating an image and/or scan, highlighting a user selected portion of an image and/or scan, adding one or more annotations to an image and/or scan, selecting a portion of an image and/or scan, and/or selecting an annotation that is overlaid onto an image and/or scan, where the selection highlights the portion and/or annotation on the respective image and/or scan.

In some embodiments, communication (e.g., the bidirectional communicating) occurs between a first facility and a second facility. In some embodiments, the first facility is one of a frozen section (FS) laboratory and an operating room (OR), and the second facility is the other of the frozen section (FS) laboratory and the operating room (OR).

In some embodiments, the communicating further includes communicating a video feed of a first user to the remote electronic device, such as a video feed of a pathologist from a frozen section laboratory to an operating room. In some embodiments, the communicating is bidirectional, and the communicating further includes communicating a video feed of a second user to the first electronic device, such as a video feed of a surgeon from an operating room to a frozen section laboratory.

In some embodiments, the communicating further includes communicating one or more additional images of the tumor specimen, or a tissue section obtained therefrom, to the remote electronic device. In some embodiments, additional images include, but are not limited to, a histopathological image, a CT scan, and/or a fluorescence image. In some embodiments, the communicating further includes communicating one or more datasets to the remote electronic device. In some embodiments, a respective dataset includes, but is not limited to, data obtained from analysis of one or more features in the tumor specimen and/or one or more tissue sections obtained therefrom (e.g., biomarkers, genes, proteins, lipids, metabolites, and/or cytokines). In some embodiments, a respective additional image in the one or more images further comprises one or more annotations.

In some embodiments, the communicating further includes communicating one or more analytic results. In some embodiments, analytic results include, but are not limited to, an indication that the tumor specimen and/or one or more tissue sections obtained therefrom have been addressed and/or discussed, an indication of a presence or absence of a supplemental tissue section (e.g., whether a close or positive margin has been addressed by obtaining a supplemental tissue section), an indication of a location of a supplemental tissue section, an indication of a size (e.g., breadth, length, and/or width) of a supplemental tissue section, and/or a type of analysis performed for a tissue section and/or a supplemental tissue section (e.g., frozen section analysis, permanent section analysis, etc.).

In some embodiments, the communicating further includes communicating one or more aspects of the tumor specimen and/or one or more tissue sections obtained therefrom as a customizable user interface (see, e.g., the section entitled “Timeout form,” below). In some embodiments, the customizable user interface includes, but is not limited to, one or more annotations, one or more images of the tumor specimen, one or more video feeds, a three-dimensional representation of the tumor specimen, a three-dimensional representation of a tumor resection defect, one or more datasets, and/or one or more analytic results.

In some embodiments, where the communicating further includes bidirectional communicating, any one or more aspects of the tumor specimen are further communicated from the remote electronic device to the first electronic device. In some embodiments, the one or more aspects include, but are not limited to, one or more annotations, one or more images of the tumor specimen, one or more video feeds, a three-dimensional representation of the tumor specimen, a three-dimensional representation of a tumor resection defect, one or more datasets, one or more indicators of analysis (e.g., frozen tissue analysis, permanent tissue analysis, biomarkers, etc.), and/or a customizable user interface.

As described above, any suitable PC or tablet that is commercially available and/or known in the art is contemplated for use as a first, second, and/or remote electronic device in the present disclosure. In some embodiments, any suitable embodiment for a first electronic device is similarly contemplated for use for a second or remote electronic device, or any substitutions or modifications thereof, as will be apparent to one skilled in the art.

Referring again to FIG. 20, in Step 3 of the example workflow for the use of three-dimensional representations of a tumor specimen and a tumor resection defect to enhance communication and documentation of tissue sections from the tumor specimen, the workflow further includes annotating the specimen scan using the lab tablet. In some embodiments, this step in the workflow marks points of interest on the surface of the specimen scan. In some implementations, this is done using commercially available “3D drawing software” on the tablet. In some embodiments, the use of a tablet advantageously provides the convenience of a direct manipulation user interface for marking points on the surface of the scan that correspond to regions of interest on the surface of the physical specimen. In some embodiments, the annotating does not include the use of a tablet.

Referring to Step 4 in FIG. 20, the example workflow further includes performing an OR/pathology lab tablet-to-tablet video conference to discuss the specimen and update the specimen scan annotations as necessary. In some embodiments, this step in the workflow comprises a discussion between the pathologist and the surgeon, to discuss the specimen, to confirm the specimen's orientation relative to the defect created in the body, and to update the locations of the points of interest, if necessary. In order that the surgeon need not break scrub and meet physically with the pathologist, in some implementations, video conferencing software advantageously connects the OR and the pathology lab. In some embodiments, the video conference is carried out from the OR using the tablet directly, or by mirroring the display of the tablet on a large video screen in the OR. An example process for Step 4 is as follows: (1) the video conference is initiated from the lab tablet; (2) the OR tablet is connected to the video conference; and (3) using the 3D annotation software on the lab tablet, the pathologist communicates the annotations made on the specimen via the video conference, and the annotations are modified in real-time as necessary until the pathologist and surgeon agree on the points of interest.

### Tumor Resection Defect Scan.

In some embodiments, method further includes obtaining a scan of the tumor resection defect that corresponds to the tumor specimen. In some embodiments, this method allows for a mapping of the tumor specimen to the tumor resection defect from which it was obtained. Generally, mapping comprises any method of associating the tumor specimen with the tumor resection defect from which it originated, and/or associating one or more tissue sections from the tumor specimen with an adjacent portion of the tumor resection defect from which it originated. In some embodiments, the mapping includes using one or more landmarks in a second plurality of landmarks. In some embodiments, the mapping does not comprise the use of landmarks.

Referring to Block 1916, in some embodiments, the method further includes obtaining a first three-dimensional representation of a tumor resection defect in the subject corresponding to the tumor specimen. In some embodiments, the first three-dimensional representation of the corresponding tumor resection defect comprises more than 1000 three-dimensional points that define a boundary of the tumor resection defect.

In some embodiments, the three-dimensional representation of the tumor resection defect comprises a plurality of three-dimensional points. In some embodiments, the plurality of three-dimensional points comprises at least 2000, at least 5000, at least 10,000, at least 20,000, at least 30,000, at least 40,000, at least 50,000, at least 100,000, at least 250,000, at least 500,000, at least 1 million, at least 1.5 million, at least 2 million, at least 3 million, or at least 5 million points. In some embodiments, the plurality of three-dimensional points comprises no more than 10 million, no more than 5 million, no more than 1 million, no more than 500,000, no more than 100,000, no more than 50,000, or no more than 10,000 points. In some embodiments, the plurality of three-dimensional points consists of from 5000 to 50,000, from 10,000 to 500,000, from 50,000 to 1 million, or from 1 million to 10 million points. In some embodiments, the plurality of three-dimensional points falls within another range starting no lower than 2000 points and ending no higher than 10 million points.

In some embodiments, the first three-dimensional representation of a tumor resection defect is obtained using one or more scans of the tumor resection defect. In some embodiments, the one or more scans comprises at least 1, at least 2, at least 3, or at least 5 scans. In some embodiments, the one or more scans comprises no more than 10, no more than 5, or no more than 2 scans. In some embodiments, the one or more scans consists of from 1 to 3, from 2 to 5, or from 5 to 10 scans. In some embodiments, the one or more scans falls within another range starting no lower than 1 scan and ending no higher than 10 scans. In some embodiments, the first three-dimensional representation of a tumor resection defect includes receiving a single scan of the tumor resection defect. In some embodiments, the one or more scans is a plurality of partial scans, and the procedure further includes combining the plurality of partial scans into the first three-dimensional representation of the tumor resection defect.

In some embodiments, the first three-dimensional representation of a tumor resection defect is obtained using a three-dimensional scanner. As an example, in some embodiments, the three-dimensional scanner used for obtaining the scan of the tumor resection defect is a defect scanner that scans the tumor resection defect and creates a computer representation of the defect, optionally referred to herein as a “3D Defect Scan.” In some embodiments, the defect scanner is located in a second facility (e.g., an operating room (OR)) that is different from the first facility. In some embodiments, the defect scanner is handheld and movable. In some embodiments, the defect scanner is capable of millimeter resolution, texture capture, and/or color capture. In some embodiments, the defect scanner is attached (e.g., via cable, communication bus 1814, and/or network communication module 1818) to a second electronic device, such as a PC and/or a tablet. In some embodiments, the second electronic device stores the 3D defect scan.

In some embodiments, the first three-dimensional representation of the corresponding tumor resection defect comprises one or more incomplete portions. Referring again to block 1916, in some implementations, the obtaining the first three-dimensional representation of the corresponding tumor resection defect in the subject further comprises determining a completion status of the first three-dimensional representation of the corresponding tumor resection defect as complete or incomplete. In some embodiments, when the completion status of the first three-dimensional representation of the corresponding tumor resection defect is incomplete, the method further includes receiving a reference representation of a defect, and mapping the first three-dimensional representation of the corresponding tumor resection defect to the reference representation of the defect, thereby correcting one or more incomplete portions of the first three-dimensional representation of the corresponding tumor resection defect.

In some embodiments, the method further includes, when a corresponding margin status of a respective tissue section satisfies a first margin status criterion, obtaining one or more supplemental specimens from the subject at a harvesting location in the tumor resection defect that is adjacent to an originating location of the respective tissue section in the tumor specimen.

In particular, referring to Block 1918, in some embodiments, the method further includes receiving a second plurality of annotations to the first three-dimensional representation of the tumor resection defect, the second plurality of annotations comprising, for each supplemental specimen in a corresponding one or more supplemental specimens, a corresponding mapping of the respective supplemental specimen to the tumor resection defect, where the one or more supplemental specimens are obtained from the subject at a location in the tumor resection defect that is adjacent to an originating location of a respective tissue section in the plurality of tissue sections that satisfies a first margin status criterion based on the corresponding margin status.

In some embodiments, the one or more supplemental specimens comprises at least 1, at least 2, at least 3, at least 4, at least 5, at least 6, at least 7, at least 8, at least 9, or at least 10 supplemental specimens. In some embodiments, the one or more supplemental specimens comprises no more than 20, no more than 10, no more than 8, no more than 5, no more than 3, no more than 2, or no more than 1 supplemental specimen. In some embodiments, the one or more supplemental specimens consists of from 1 to 3, from 2 to 5, from 3 to 10, or from 8 to 20 supplemental specimens. In some embodiments, the one or more supplemental specimens falls within another range starting no lower than 1 supplemental specimen and ending no higher than 20 supplemental specimens.

In some embodiments, the second plurality of annotations comprises at least 2, at least 3, at least 4, at least 5, at least 6, at least 7, at least 8, at least 9, at least 10, at least 20, or at least 50 annotations. In some embodiments, the second plurality of annotations comprises no more than 100, no more than 30, no more than 20, no more than 10, no more than 8, no more than 5, no more than 3 annotations. In some embodiments, the second plurality of annotations consists of from 2 to 10, from 5 to 20, from 10 to 30, or from 25 to 100 annotations. In some embodiments, the second plurality of annotations falls within another range starting no lower than 2 annotations and ending no higher than 100 annotations.

In some embodiments, the first margin status criterion is satisfied when the respective tissue section has a close or positive margin based upon a pathological analysis of the respective tissue section. In this way, in some implementations, the method includes harvesting additional samples from the tumor resection defect that correspond to regions where the margins of the tumor specimen are deemed to be at risk (e.g., close or positive margins).

In some embodiments, the mapping of the respective supplemental specimen to the tumor resection defect indicates a position, a breadth, a length, and/or an orientation of the respective supplemental specimen relative to the tumor resection defect (e.g., from which the supplemental specimen was harvested).

In some embodiments, a respective annotation in the second plurality of annotations comprises a corresponding label for the respective supplemental specimen. In some embodiments, the label is a number (e.g., 1, 2, 3, 4, etc.), an alphanumeric string, an asterisk, an arrow, a geometric shape, and/or any other suitable symbol or identifier. In some embodiments, a respective annotation in the second plurality of annotations comprises a delineator between a first supplemental specimen and a second supplemental specimen (e.g., a line showing where supplemental specimen are harvested from the tumor resection defect). In some embodiments, one or more annotations in the second plurality of annotations are added manually (e.g., using a mouse or a stylus on a device, such as a computer or a tablet). In some embodiments, one or more annotations in the second plurality of annotations are performed using a touchscreen device. In some embodiments, one or more annotations in the second plurality of annotations are added to the 3D representation based on an indication of a region of the 3D representation with which a respective annotation is associated (e.g., a text label assigned to a coordinate position in the 3D representation). In some embodiments, one or more annotations are added using automation. Any suitable embodiment disclosed herein for a first plurality of annotations is further contemplated for use for a second plurality of annotations, as well as any substitutions, combinations, or modifications thereof, as will be apparent to one skilled in the art (see, for instance, the section entitled “Tumor specimen scan,” above).

In some embodiments, the method further includes, prior to the harvesting, determining an orientation of the tumor specimen relative to a corresponding tumor resection defect in the subject. In some embodiments, the orientation is determined by a procedure comprising determining a second plurality of landmarks. In some such embodiments, each respective landmark in the second plurality of landmarks comprises a corresponding pair of reference positions including a first respective reference position for the first three-dimensional representation of the tumor specimen and a second respective reference position for the first three-dimensional representation of the corresponding tumor resection defect, where the first and second reference positions are positioned at corresponding features in the tumor specimen and the tumor resection defect. Thus, a third set of landmark coordinates for the first three-dimensional representation of the tumor specimen and a fourth set of landmark coordinates for the first three-dimensional representation of the corresponding tumor resection defect are identified. In some embodiments, the third set of landmark coordinates and the fourth set of landmark coordinates are used to obtain an alignment of the first three-dimensional representation of the tumor specimen with the first three-dimensional representation of the corresponding tumor resection defect. Any suitable embodiment disclosed herein for a first plurality of landmarks is further contemplated for use for a second plurality of landmarks, as will be apparent to one skilled in the art (see, for instance, the section entitled “Tumor specimen scan,” above).

In some embodiments, the first three-dimensional representation of the corresponding tumor resection defect is received from a remote electronic device. For example, in some implementations, the 3D defect scan is received at the first electronic device (e.g., in a pathology or FS laboratory) from a remote electronic device (e.g., in an operating room). In some embodiments, the method further includes displaying, after the receiving, at the first electronic device, a second three-dimensional representation of the corresponding tumor resection defect, and applying, responsive to a first user interaction with respect to the first three-dimensional representation of the corresponding tumor resection defect at the remote electronic device, the first user interaction to the second three-dimensional representation of the corresponding tumor resection defect at the first electronic device. In some embodiments, the second three-dimensional representation of the corresponding tumor resection defect is a composite three-dimensional representation of the tumor resection defect comprising the first three-dimensional representation of the tumor resection defect modified to include the second plurality of annotations.

In some embodiments, the first user interaction is selected from the group consisting of changing a scale of the composite three-dimensional representation, panning the composite three-dimensional representation, rotating composite three-dimensional representation, and highlighting a user selected portion of the composite three-dimensional representation. In some embodiments, the first user interaction comprises adding one or more annotations to the first three-dimensional representation of the tumor specimen.

Thus, in some implementations, the first three-dimensional representation of the tumor resection defect, the second plurality of annotations, and/or the composite three-dimensional representation thereof are interactively communicated (e.g., can be generated or manipulated via user interaction, which is transmitted in real-time to a first electronic device from a remote device). In some embodiments, the communicating is bidirectional. In some such embodiments, one or more user interactions with the second three-dimensional representation of the tumor resection defect at the first electronic device are further communicated and/or displayed at the remote electronic device. Bidirectional communication contemplated for use in the present disclosure is further discussed elsewhere herein (see, for instance, the section entitled “Communication of three-dimensional representations,” above).

Referring again to FIG. 20, in Step 5 of the example workflow for the use of three-dimensional representations of a tumor specimen and a tumor resection defect to enhance communication and documentation of tissue sections from the tumor specimen, the workflow further includes creating a defect scan using an operating room (OR) scanner. In the example workflow, the operating room is set up prior to scanning. In some embodiments, the scanning hardware used in the OR is different than that used in the laboratory because the defect is scanned in situ. In some embodiments, the defect is scanned using a handheld 3D scanner that can be moved in space around the defect until the entire surface of the defect has been scanned. In some embodiments, the defect scanner is further connected (e.g., via cable, communication bus 1814, and/or network communication 1818) to a companion PC that is located on a wheeled cart so that the scanner can be positioned as necessary. In some embodiments, neither the scanner nor the PC are sterilized, and the scanning is done by a technician that has not been scrubbed into the OR and remains nonsterile throughout the scanning process. In some embodiments, the scanner and the PC rest on a moveable surface (e.g., a surgical cart) that can be moved near the defect without interfering with the surgical setup. In some implementations, the scanner and laptop are positioned so that the entire defect can be scanned with an appropriate range of motion without contacting sterile instrument tables. In some embodiments, a footstool is used while scanning to achieve a better angle. In some implementations, the OR overhead lights are adjusted in order not to interfere with the scanning process. In some implementations, any surgical towel or drapes are also repositioned so that they do not obscure the edges of the defect where margins will be assessed. In some embodiments, the angle of the table can be adjusted in order to achieve a direct view of the defect by the scanner without the need to pass the scanner over the sterile field. An example protocol for obtaining a defect scan is as follows: (1) the scanner is held in the dominant hand and positioned perpendicularly to the defect surface, while the scanner cord is held in the other hand, making sure it does not contaminate the sterile field or get entangled; (2) the field brightness is adjusted using a companion PC application and the OR lights are repositioned as necessary; (3) the scan is initiated, and the progress of the scan is monitored using the companion application on the PC; and (4) the scan is continued until the entire surface of the defect has been scanned, taking care to maintain an appropriate distance of the scanner from the defect. In some embodiments, the display of the connected PC is mirrored on a portable electronic device such as a smartphone or tablet, allowing the individual performing the scan to see the progress of the scan without having to divert attention away from the scanning process itself.

Referring to Step 6 in FIG. 20, the example workflow further includes uploading the defect scan to an OR tablet. For instance, once the defect scan is completed, it is transferred to the tablet, allowing for subsequent annotation by a surgeon in the OR. In some implementations, annotations include marking locations on the boundary of the defect that denote the precise location and breadth of each supplemental specimen to be processed in the frozen section room. An example protocol for uploading the defect scan is as follows: (1) on the OR PC, the underlying computer representation of the defect scan is transformed to one that the software on the OR Tablet is capable of processing; (2) on the OR PC, the resulting scan is saved to a network folder that is accessible from the OR tablet; and (3) on the OR tablet, the specimen scan is uploaded from the network folder.

### Timeout Form.

In some embodiments, a customizable user interface is used for single- or bidirectional communication and documentation of the tumor specimen scan, the defect scan, and/or surgical margin analysis thereof.

Referring to Block 1920, in some embodiments, the method further includes displaying, on the display of the first electronic device, a customizable user interface comprising, for each respective tissue section in the corresponding plurality of tissue sections from the tumor specimen, one or more affordances. In some embodiments, the one or more affordances includes a first affordance for providing a first annotation that indicates an identity of the tumor specimen, a second affordance for providing a second annotation that indicates an identity of the respective tissue section, a third affordance for providing a third annotation that provides a corresponding margin status for the respective tissue section, and/or a fourth affordance for updating a review status of the respective tissue section. In some embodiments, the customizable user interface comprises a line-item table.

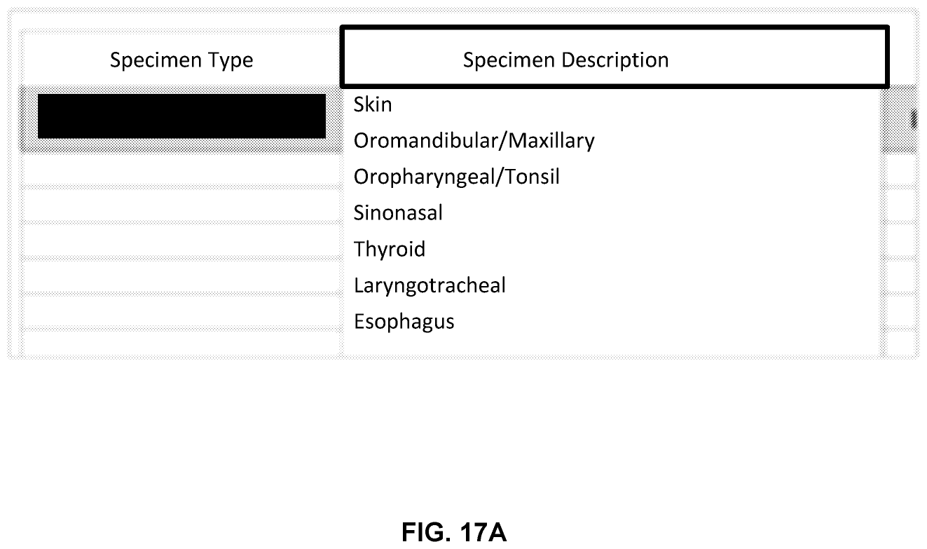

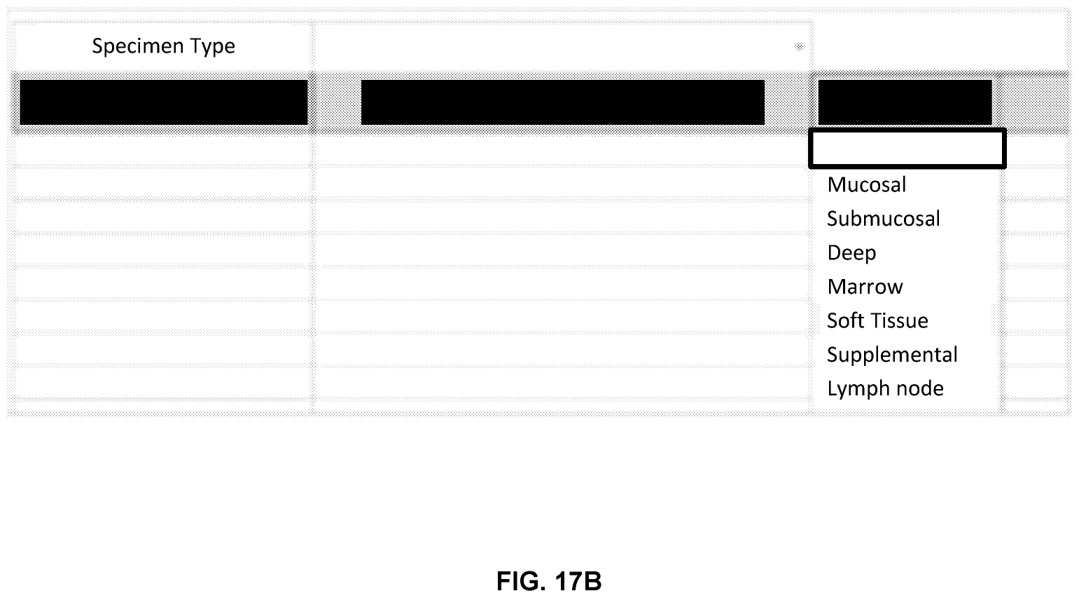

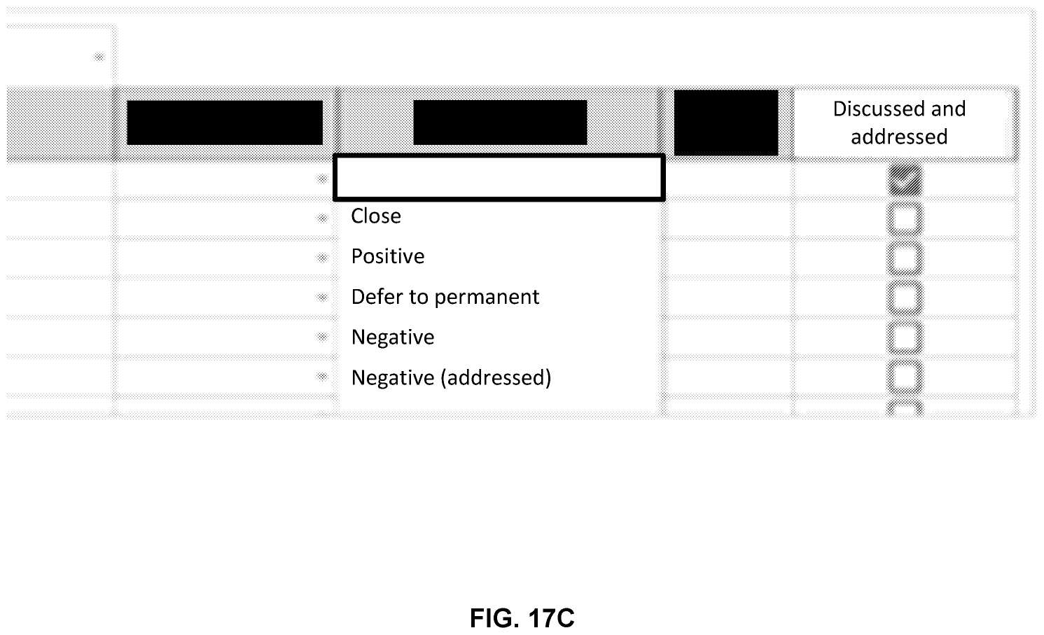

FIGS. 17A-C collectively illustrate an example of a customizable user interface in accordance with some embodiments of the present disclosure. FIG. 17A illustrates at least a first affordance for providing a first annotation that indicates an identity of a tumor specimen. In some implementations, the first affordance is selected from a specimen type and/or a specimen description (e.g., skin, oromandibular, maxillary, oropharyngeal, tonsil, sinonasal, thyroid, laryngotracheal, and/or esophagus). FIG. 17B illustrates at least a second affordance for providing a second annotation that indicates an identity of a respective tissue section. In some implementations, the second affordance is selected from a specimen margin ID and/or a margin type (e.g., mucosal, submucosal, deep, marrow, soft tissue, supplemental, and/or lymph node). FIG. 17C illustrates at least a third affordance for providing a third annotation that provides a corresponding margin status for a respective tissue section. In some implementations, the third affordance comprises a margin status (e.g., close, positive, defer to permanent, negative, and/or negative (addressed)) and/or a margin breadth (e.g., in millimeters). FIG. 17C further illustrates a fourth affordance for updating a review status of a respective tissue section (e.g., discussed and/or addressed).

In some embodiments, upon user selection of the fourth affordance, the review status of the respective tissue section is updated from a first review status to a second review status (e.g., “not discussed or addressed” to “discussed and/or addressed”).

In some embodiments, the customizable user interface further comprises a fifth affordance for appending one or more supplemental specimens to the plurality of tissue sections. In some embodiments, when a corresponding margin status for a respective tissue section in the plurality of tissue sections satisfies a first margin status criterion, the method further includes obtaining one or more supplemental specimens from the subject at a location in the tumor resection defect that is adjacent to an originating location of the respective tissue section; appending, upon user selection of the fifth affordance, the one or more supplemental specimens to the plurality of tissue sections; updating, upon user selection of the fourth affordance, the review status of the respective tissue section from a first review status to a second review status; and, for each respective supplemental specimen in the one or more supplemental specimens, indicating, upon user selection of the second affordance, (i) an identity of the respective supplemental specimen and (ii) an identity of the respective tissue section in the plurality of tissue sections.

In some embodiments, the first margin status criterion is satisfied when the respective tissue section has a close or positive margin based upon a pathological analysis of the respective tissue section. As described above, in some implementations, additional samples are harvested from the tumor resection defect that correspond to regions where the margins of the tumor specimen are deemed to be at risk (e.g., close or positive margins). The customizable user interface is then updated to indicate that the respective tissue section has been discussed and/or addressed (e.g., by harvesting the supplemental specimen, performing a frozen section analysis, and/or discussing the results of the frozen section analysis). Moreover, the customizable user interface is updated to add the supplemental specimen to the list of tissue sections, indicate the corresponding at-risk tissue section with which it is associated, and/or indicate a margin status and/or further analysis of the supplemental specimen.

In some embodiments, the customizable user interface further includes one or more images, scans, and/or three-dimensional representations of the tumor specimen and/or the tumor resection defect. In some embodiments, the customizable user interface is displayed concurrently (e.g., on a first, second, and/or remote electronic device) with one or more images, scans, and/or three-dimensional representations. Examples of images, scans, and/or three-dimensional representations that can be included in the customizable user interface include, but are not limited to, 3D scans, CT scans, histopathological sections, and/or fluorescent images. Alternatively or additionally, in some embodiments, the customizable user interface further includes a video feed (e.g., of a first user at the first electronic device to a remote electronic device, and/or of a second user at a remote electronic device to the first electronic device). In some embodiments, the customizable user interface is displayed concurrently with a video feed (e.g., of a first user at the first electronic device to a remote electronic device, and/or of a second user at a remote electronic device to the first electronic device). Alternatively or additionally, in some embodiments, the customizable user interface further includes one or more datasets and/or one or more analytic results, as described elsewhere herein (e.g., frozen tissue analysis, permanent tissue analysis, biomarkers, etc.; see, for example, the section entitled “Communication of three-dimensional representations,” above). In some embodiments, the customizable user interface is displayed concurrently with one or more datasets and/or one or more indicators of analysis.

In some embodiments, the customizable user interface is interactive. For instance, in some embodiments, a user manipulates the customizable user interface via a user interaction. In some embodiments, the user interaction modifies the customizable user interface, and the user interaction is displayed on the display (e.g., of the first electronic device). As described above, suitable embodiments for user interactions, include, but are not limited to, changing a scale of an image and/or scan, panning an image and/or scan, rotating an image and/or scan, highlighting a user selected portion of an image and/or scan, adding one or more annotations to an image and/or scan, selecting or isolating a portion of an image and/or scan, and/or selecting or isolating an annotation that is overlaid onto an image and/or scan, where the selection or isolation highlights the portion and/or annotation on the respective image and/or scan.

In some embodiments, one or more components of the customizable user interface are linked (e.g., a line-item table, an image, a scan, a three-dimensional representation, an annotation, a video feed, a dataset, and/or an analytic result). In some embodiments, a respective user interaction with one of a first respective component and a second respective component of the customizable user interface further modifies the other of the first respective component and the second respective component, where the first component and the second component are linked. In some implementations, the first component is a respective annotation (e.g., in the first and/or second plurality of annotations) that is overlaid on a respective three-dimensional representation and the second component is an entry in a line-item table of the customizable user interface. For example, in some embodiments, selection of an annotation that is overlaid on a 3D specimen scan highlights both the annotation overlay on the 3D specimen scan and the corresponding entry in the line-item table of the customizable user interface that states the annotation in text form. In some such embodiments, the annotation is a manually inputted (e.g., hand-drawn) annotation indicating a particular tissue section in a tumor specimen scan, and the entry in the line-item table is a corresponding row in the line-item table, or a portion thereof, that corresponds to the particular tissue section. Alternatively or additionally, in some embodiments, selection of an entry in a line-item table of a customizable user interface highlights both the entry and a corresponding annotation that is overlaid on the specimen scan. In this way, various corresponding portions of the customizable user interface and/or associated images or datasets can be viewed or referenced concurrently by user interaction. In some embodiments, two or more, three or more, four or more, or five or more components of the customizable user interface are linked. For example, in some embodiments, user interaction with a first component of the customizable user interface concurrently performs selects and/or modifies at least a second and a third component of the customizable user interface (e.g., where an entry for a supplemental specimen in the line-item table is selected, a corresponding tissue section in the specimen scan and a corresponding annotation in the defect scan are similarly selected).

In some embodiments, the method further includes communicating one or more user interface instructions to a remote electronic device, where the one or more user interface instructions comprises (i) instructions for displaying an image overlay on the remote electronic device comprising the customizable user interface, and (ii) instructions that, responsive to a first user interaction with respect to the customizable user interface at the first electronic device, apply the first user interaction to the customizable user interface at the remote electronic device. In some such embodiments, the customizable user interface and/or the one or more images, scans, and/or three-dimensional representations are displayed and/or are user-interactive on one or both of a first electronic device and a second (e.g., remote) electronic device. Accordingly, in some implementations, any user interaction (e.g., as described above) made to the customizable user interface from the first electronic device is communicated to the second electronic device, and/or any user interaction made to the customizable user interface from the second electronic device is communicated to the first electronic device.

Referring again to FIG. 20, in Step 7 of the example workflow for the use of three-dimensional representations of a tumor specimen and a tumor resection defect to enhance communication and documentation of tissue sections from the tumor specimen, the workflow further includes creating a customizable user interface, optionally referred to herein as a “timeout form,” that describes one or more initial margin results and one or more views (e.g., screenshots) of the annotated specimen scan. In some embodiments, the timeout form is a spreadsheet that can be edited using a standalone tool and/or a collaborative spreadsheet editor. In some implementations, a collaborative editor is used to allow multiple collaborators to work on it at the same time and to allow edits to be seen in real time. In some embodiments, at the top of the spreadsheet are spaces designated to record the date of procedure, patient name, patient's date of birth, diagnosis, procedure being performed, and/or the surgeons involved. In some embodiments, each row of the spreadsheet corresponds to a margin analysis and specimen, and the specific annotation on the defect scan associates the location of the specimen (on the defect), and the specific margin analyses performed on that specimen. In some embodiments, once the specimen is positioned in the exact orientation that is desired for a still image, pictures (e.g., JPEG or PNG) are exported from the tablet annotation software and saved into a cell that identifies a row of the timeout form.

In some embodiments, frozen section results are displayed as a line-item table. For example, as frozen section margins are analyzed by a pathologist, they are transcribed into the frozen section timeout form for documentation. In some implementations, referring again to FIGS. 17A-C, the pathologist uses dropdown cells and menu options to select various preset options for the pathology results of each tissue section (e.g., cassette sample). In some embodiments, the spreadsheet is customized so that the specific “margin type” options change depending on which “specimen type” is selected, adapting the form to the specific procedure being conducted. In some implementations, the status of each margin is recorded as “close,” “positive,” “negative.” In some embodiments, when “close” or “positive” are selected, the form automatically formats the cell by highlighting it in red to draw attention to a margin at risk. In some embodiments, when a margin is deemed “close,” a column is available to record the tumor's distance from the closest margin.

Referring to Step 8 in FIG. 20, the example workflow further includes performing an OR/lab tablet-to-tablet video conference to discuss the margin results. In some embodiments, once pathology gross analysis is complete, the surgeon and pathologist use a video conference to discuss margin results. In some embodiments, the pathologist communicates margin results by sharing the timeout form, including at least a line-item table and/or annotated specimen scans. In some implementations, the surgeon uses the communicated timeout form and/or annotated specimen scans to evaluate whether, based on margin results, additional supplemental margins will be harvested. In some implementations, when additional supplemental specimens are harvested, additional rows are added to the timeout form. In some implementations where additional supplemental tissue is harvested from the defect to address a margin at risk, an option reading “negative (addressed)” is used. In some implementations, the surgeon annotates the precise location and size of the supplemental tissue on the defect scan where the additional specimen is harvested, and creates an additional row in the timeout form. In some implementations, a visual cue is used (e.g., changing a color of the at-risk margin, such as from red or pink to green) to document that the close margin has been addressed. In some implementations, the timeout form uses a nested notation to indicate that the supplemental tissue addresses a previously indicated margin at risk (e.g., “Specimen ID of Tissue (Previous Specimen ID of Tissue Being Addressed)”).

Referring to Step 9 in FIG. 20, the example workflow further includes annotating the defect scan to describe the supplemental margins. In some implementations, after the defect scan is imported into the OR tablet, the surgeon utilizes an overglove to intraoperatively annotate the defect. Annotations usually demarcate the location and breadth of supplemental margins harvested.

Referring to Step 10 in FIG. 20, the example workflow further includes updating the timeout form with supplemental margin results and/or annotated defect scan views (e.g., screenshots). In some implementations, the defect scan is augmented with reference or “textbook” defect scans to compensate for any defect scan quality issues, if necessary. Such reference defect scans are advantageously used to accommodate hardware limitations. For instance, depending on the surgical procedure, the defect scanner may not be able to scan the entire surface of the defect. Thus, in some implementations, when a defect contains a cavity that is too small or inaccessible for the scanner to capture, a gap or incomplete portion is present in the 3D scan of the defect surface. In order to compensate for this, in some implementations, reference or “textbook” images of the defect and/or various organs are used to complete the incomplete portion. In some embodiments, the reference image is further added as a row of the timeout form, thus allowing the specific harvesting location to be properly documented, even if the defect scan is incomplete.

Referring to Step 11 in FIG. 20, the example workflow further includes performing a final OR/lab video conference in order to reconcile all of the margin results. At this point in the surgery the margins have all been analyzed, and a final video conference is taken between the OR and frozen section room, optionally referred to herein as the “pathology timeout.” In some embodiments, the pathology timeout is performed to ensure that nothing has been missed and that all member of the healthcare team receive the same description of the results. An example process for the pathology timeout is as follows: (1) the surgeon and pathologist lead a final video conference to reconcile margin statuses and ensure all members of the healthcare team are on the same page; (2) the pathologist shares the completed timeout form and summarizes margin results, documenting in the form that each margin is marked as “Discussed and Addressed;” (3) the surgeon uses the annotated defect scan to explain where and how supplemental margins were taken; and (4) following the pathology timeout, the pathologist will note on the form either that all margins have been successfully addressed or that a suspicious margin remains unaddressed.

### Reporting.

Referring to Block 1922, in some embodiments, the method further includes generating a report for the subject using at least the first three-dimensional representation of the tumor specimen and the first plurality of annotations.

In some embodiments, the report is an electronic health record (EHR).

In some embodiments, the report further includes the first three-dimensional representation of the tumor resection defect, the second plurality of annotations, the customizable user interface (e.g., the timeout form), and/or any component, aspect, or combination thereof.

In some implementations, the report further includes one or more additional images of the tumor specimen and/or the tumor resection defect, including, but not limited to, a histopathological image, a CT scan, and/or a fluorescence image.

In some implementations, the report further includes one or more datasets including, but not limited to, data obtained from analysis of one or more features in the tumor specimen and/or one or more tissue sections obtained therefrom (e.g., biomarkers, genes, proteins, lipids, metabolites, and/or cytokines).

In some implementations, the report further includes one or more analytic results. Alternatively or additionally, in some embodiments, the report further includes permanent section results. In some implementations, the report further includes one or more notes or comments appended to any of the components of the report disclosed herein. In some implementations, the report further includes medical information (e.g., date of surgery, date of consultation, subject information, surgeon and/or pathologist information, procedural information, and/or medical practitioner signature).

In some implementations, the report further includes one or more video clips, such as a clip of a video feed between a frozen section laboratory and an operating room.

In some embodiments, the report is interactive. In some embodiments, the report is in multimedia format. Suitable embodiments for user interactions, include, but are not limited to, changing a scale of one or more components of the report, panning one or more components of the report, rotating one or more components of the report, highlighting a user selected portion of the report, adding one or more annotations to the report, selecting a portion of the report, and/or selecting an annotation that is included or overlaid onto one or more components of the report, where the selection highlights the respective portion and/or the respective annotation in the report.

Any of the embodiments disclosed herein for user interactions (e.g., for a customizable user interface, a three-dimensional representation, an image, and/or a scan) are similarly contemplated for use with the report, or any components or embodiments thereof. For instance, in some embodiments, the report comprises linked components. In some implementations, one or more components of the report (e.g., a line-item table, an image, a scan, a three-dimensional representation, an annotation, a video feed, a dataset, and/or an analytic result) are linked with one or more other components of the report. In some embodiments, a respective user interaction with one of a first respective component and a second respective component of the report further modifies the other of the first respective component and the second respective component, where the first component and the second component are linked.

Advantageously, in some implementations, the report creates a permanent record in the form of a novel final pathology report of the surgery, for use by downstream practitioners, such as radiation oncologists, medical oncologists, and/or radiologists. In some implementations, the report is further used or referenced at a later timepoint in the subject's medical treatment, such as in the event of subsequent surgeries.

Referring again to FIG. 20, in Step 12 of the example workflow for the use of three-dimensional representations of a tumor specimen and a tumor resection defect to enhance communication and documentation of tissue sections from the tumor specimen, the workflow further includes creating a final pathology lab report, optionally with CT scan, margin results from the timeout form, and/or permanent section results. In particular, in some embodiments, after the procedure has finished, the timeout form is converted into a final pathology report that further includes pertinent medical information and permanent pathology results. In some implementations, this report is used to communicate results with patients and other healthcare professionals. An example process for generating a report is as follows: (1) the patient's CT scans are obtained from the medical record database and relevant scans are inserted into the timeout form, where radiographic scans are optionally annotated either in axial, coronal or sagittal orientation as to where supplemental margins were harvested so that all care providers can more readily pinpoint on follow up scans where problem areas were located; (2) permanent section pathology results are searched in the medical record or pathology database a few days after the procedure such that, once they have been uploaded, the results are included in the report; and (3) the final pathology report is uploaded into the database or printed out for reference.

Advantageously, in some embodiments, the presently disclosed systems and methods reduce anesthesia duration during surgery by reducing the need for surgeons to scrub out during surgery (e.g., for intraoperative consultation). See, for example, the section entitled “Introduction,” above. Increased anesthesia duration has been associated with increased rates of complications in some cases. Accordingly, in some embodiments, the presently disclosed systems and methods advantageously reduce rates of postoperative complications.

Additional advantages include, but are not limited to, more precise resection of supplemental margins (e.g., supplemental specimens) from the tumor resection defect due to enhanced mapping and more comprehensive cancer resection resulting from that precise mapping process. In addition, in some implementations, this precise mapping and three-dimensional documentation enhances postoperative adjuvant radiation targeting of close or positive margins. In some implementations, the presently disclosed systems and methods are utilized for more accurate interpretation of post-treatment surveillance scans with possible earlier detection of disease recurrence.

Another aspect of the present disclosure provides a method for localizing close or positive margins in a tumor specimen from a subject. In some embodiments, the method comprises, at a first electronic device with a display and an input device. In some embodiments, the method includes obtaining a first three-dimensional representation of the tumor specimen. In some embodiments, the three-dimensional representation comprises more than 1000 three-dimensional points that define a boundary of the tumor specimen. A first plurality of annotations to the first three-dimensional representation of the tumor specimen is received. The first plurality of annotations comprises a first subset of annotations, each respective annotation in the first subset of annotations indicating an orientation of the first three-dimensional representation of the tumor specimen relative to the subject, a second subset of annotations, each respective annotation in the second subset of annotations indicating a respective tissue section in a corresponding plurality of tissue sections from the tumor specimen, and a third subset of annotations, each respective annotation in the third subset of annotations providing a corresponding margin status for a respective tissue section in the plurality of tissue sections, where the corresponding margin status indicates whether the respective tissue section has a close or positive margin based upon a pathological analysis of the respective tissue section. A composite three-dimensional representation of the tumor specimen is formed by modifying the first three-dimensional representation of the tumor specimen to include the first plurality of annotations.

Another aspect of the present disclosure provides a non-transitory computer-readable storage medium having stored thereon program code instructions that, when executed by a processor, cause the processor to perform a method for localizing close or positive margins in a tumor specimen from a subject. In some embodiments, the method includes obtaining a first three-dimensional representation of the tumor specimen. In some embodiments, the three-dimensional representation comprises more than 1000 three-dimensional points that define a boundary of the tumor specimen. A first plurality of annotations to the first three-dimensional representation of the tumor specimen is received. The first plurality of annotations comprises a first subset of annotations, each respective annotation in the first subset of annotations indicating an orientation of the first three-dimensional representation of the tumor specimen relative to the subject, a second subset of annotations, each respective annotation in the second subset of annotations indicating a respective tissue section in a corresponding plurality of tissue sections from the tumor specimen, and a third subset of annotations, each respective annotation in the third subset of annotations providing a corresponding margin status for a respective tissue section in the plurality of tissue sections, where the corresponding margin status indicates whether the respective tissue section has a close or positive margin based upon a pathological analysis of the respective tissue section. A composite three-dimensional representation of the tumor specimen is formed by modifying the first three-dimensional representation of the tumor specimen to include the first plurality of annotations.

### ADDITIONAL EMBODIMENTS

Still another aspect of the present disclosure provides a method for localizing close or positive margins in a tumor specimen from a subject. In some embodiments, the method is performed at a first electronic device with a display and an input device (e.g., in an operating room (OR)). In some embodiments, the method includes receiving, from a second electronic device (e.g., in a frozen section (FS) laboratory) (i) a first three-dimensional representation of the tumor specimen. In some such embodiments, the first three-dimensional representation comprises more than 1000 three-dimensional points that define a boundary of the tumor specimen. In some embodiments, there is further received (ii) a first plurality of annotations to the first three-dimensional representation of the tumor specimen, the first plurality of annotations comprising a first subset of annotations, each respective annotation in the first subset of annotations indicating an orientation of the first three-dimensional representation of the tumor specimen relative to the subject; a second subset of annotations, each respective annotation in the second subset of annotations indicating a respective tissue section in a corresponding plurality of tissue sections from the tumor specimen; and a third subset of annotations, each respective annotation in the third subset of annotations providing a corresponding margin status for a respective tissue section in the plurality of tissue sections, where the corresponding margin status indicates whether the respective tissue section has a close or positive margin based upon a pathological analysis of the respective tissue section. In some embodiments, there is further received (iii) one or more composite three-dimensional representation instructions. In some embodiments, the method further includes displaying, after the receiving (i) a composite three-dimensional representation of the tumor specimen in the form of the first plurality of annotations and the first three-dimensional representation of the tumor specimen, thereby producing a second three-dimensional representation of the tumor specimen, and (ii) responsive to a user interaction with respect to the composite three-dimensional representation of the tumor specimen at the second electronic device, the user interaction applied to the second three-dimensional representation of the tumor specimen.

In some embodiments, the method further includes, at the first electronic device, obtaining a first three-dimensional representation of a corresponding tumor resection defect. One or more tumor resection defect instructions are communicated to the second electronic device (e.g., from an OR to an FS lab), where the one or more tumor resection defect instructions comprises (i) instructions for displaying an image overlay on the second electronic device comprising the first three-dimensional representation of the corresponding tumor resection defect, thereby producing a second three-dimensional representation of the corresponding tumor resection defect, and (ii) instructions that, responsive to a first user interaction with respect to the three-dimensional representation of the corresponding tumor resection defect at the first electronic device, apply the first user interaction to the second three-dimensional representation of the corresponding tumor resection defect at the second electronic device.

In some embodiments, the method further includes, when a corresponding margin status of a respective tissue section satisfies a first margin status criterion, obtaining one or more supplemental specimens from the subject at a harvesting location in the tumor resection defect that is adjacent to an originating location of the respective tissue section in the tumor specimen, and receiving a second plurality of annotations to the first three-dimensional representation of the tumor resection defect. In some embodiments, the second plurality of annotations comprises, for each supplemental specimen in the one or more supplemental specimens, a corresponding mapping of the respective supplemental specimen to the tumor resection defect.

In some embodiments, the second three-dimensional representation of the corresponding tumor resection defect on the second electronic device is a composite three-dimensional representation of the tumor resection defect comprising the first three-dimensional representation of the tumor resection defect modified to include the second plurality of annotations.

In some embodiments, the method further includes displaying, on the display of the first electronic device, a customizable user interface comprising, for each respective tissue section in the corresponding plurality of tissue sections from the tumor specimen, a first affordance for providing a first annotation that indicates an identity of the tumor specimen, a second affordance for providing a second annotation that indicates an identity of the respective tissue section, a third affordance for providing a third annotation that provides a corresponding margin status for the respective tissue section, and a fourth affordance for updating a review status of the respective tissue section.

In some embodiments, the method further includes communicating one or more user interface instructions to the second electronic device, where the one or more user interface instructions comprises (i) instructions for displaying an image overlay on the remote electronic device comprising the customizable user interface, and (ii) instructions that, responsive to a first user interaction with respect to the customizable user interface at the first electronic device, apply the first user interaction to the customizable user interface at the second electronic device.

Another aspect of the present disclosure provides an electronic device, comprising a display; an input device; one or more processors; memory; and one or more programs, where the one or more programs are stored in the memory and configured to be executed by the one or more processors, the one or more programs including instructions for a method for localizing close or positive margins in a tumor specimen from a subject.

In some embodiments, the method includes receiving, from a second electronic device (i) a first three-dimensional representation of the tumor specimen. In some such embodiments, the first three-dimensional representation comprises more than 1000 three-dimensional points that define a boundary of the tumor specimen. In some embodiments, there is further received (ii) a first plurality of annotations to the first three-dimensional representation of the tumor specimen, the first plurality of annotations comprising a first subset of annotations, each respective annotation in the first subset of annotations indicating an orientation of the first three-dimensional representation of the tumor specimen relative to the subject; a second subset of annotations, each respective annotation in the second subset of annotations indicating a respective tissue section in a corresponding plurality of tissue sections from the tumor specimen; and a third subset of annotations, each respective annotation in the third subset of annotations providing a corresponding margin status for a respective tissue section in the plurality of tissue sections, where the corresponding margin status indicates whether the respective tissue section has a close or positive margin based upon a pathological analysis of the respective tissue section. In some embodiments, there is further received (iii) one or more composite three-dimensional representation instructions. In some embodiments, the method further includes displaying, after the receiving (i) a composite three-dimensional representation of the tumor specimen in the form of the first plurality of annotations and the first three-dimensional representation of the tumor specimen, thereby producing a second three-dimensional representation of the tumor specimen, and (ii) responsive to a user interaction with respect to the composite three-dimensional representation of the tumor specimen at the second electronic device, the user interaction applied to the second three-dimensional representation of the tumor specimen.

Another aspect of the present disclosure provides a non-transitory computer-readable storage medium having stored thereon program code instructions that, when executed by a processor, cause the processor to perform a method for localizing close or positive margins in a tumor specimen from a subject.

In some embodiments, the method includes receiving, from a second electronic device (i) a first three-dimensional representation of the tumor specimen. In some such embodiments, the first three-dimensional representation comprises more than 1000 three-dimensional points that define a boundary of the tumor specimen. In some embodiments, there is further received (ii) a first plurality of annotations to the first three-dimensional representation of the tumor specimen, the first plurality of annotations comprising a first subset of annotations, each respective annotation in the first subset of annotations indicating an orientation of the first three-dimensional representation of the tumor specimen relative to the subject; a second subset of annotations, each respective annotation in the second subset of annotations indicating a respective tissue section in a corresponding plurality of tissue sections from the tumor specimen; and a third subset of annotations, each respective annotation in the third subset of annotations providing a corresponding margin status for a respective tissue section in the plurality of tissue sections, where the corresponding margin status indicates whether the respective tissue section has a close or positive margin based upon a pathological analysis of the respective tissue section. In some embodiments, there is further received (iii) one or more composite three-dimensional representation instructions. In some embodiments, the method further includes displaying, after the receiving (i) a composite three-dimensional representation of the tumor specimen in the form of the first plurality of annotations and the first three-dimensional representation of the tumor specimen, thereby producing a second three-dimensional representation of the tumor specimen, and (ii) responsive to a user interaction with respect to the composite three-dimensional representation of the tumor specimen at the second electronic device, the user interaction applied to the second three-dimensional representation of the tumor specimen.

Yet another aspect of the present disclosure provides a method for localizing a tissue section in a tumor specimen from a subject to a corresponding tumor resection defect in the subject. In some embodiments, the method is performed at an electronic device with a display and an input device. In some embodiments, the method includes obtaining a first three-dimensional representation of the tumor specimen. In some embodiments, the three-dimensional representation comprises more than 1000 three-dimensional points that define a boundary of the tumor specimen. A first plurality of annotations to the first three-dimensional representation of the tumor specimen is received, the first plurality of annotations comprising a first subset of annotations, each respective annotation in the first subset of annotations indicating an orientation of the first three-dimensional representation of the tumor specimen relative to the subject, a second subset of annotations, each respective annotation in the second subset of annotations indicating a respective tissue section in a corresponding plurality of tissue sections from the tumor specimen, and a third subset of annotations, each respective annotation in the third subset of annotations providing a corresponding margin status for a respective tissue section in the plurality of tissue sections, where the corresponding margin status indicates whether the respective tissue section has a close or positive margin based upon a pathological analysis of the respective tissue section. A first three-dimensional representation of a corresponding tumor resection defect in the subject is obtained. In some embodiments, the first three-dimensional representation of the corresponding tumor resection defect comprises more than 1000 three-dimensional points that define a boundary of the tumor resection defect. In some embodiments, the method further includes determining an orientation of the tumor specimen relative to the corresponding tumor resection defect, and applying, when a corresponding margin status of a respective tissue section in the plurality of tissue sections satisfies a first margin status criterion, a second plurality of annotations to the first three-dimensional representation of the tumor resection defect. In some embodiments, the second plurality of annotations comprises, for each respective supplemental specimen in one or more supplemental specimens associated with the respective tissue section, a corresponding mapping of the respective supplemental specimen to the tumor resection defect.

In some embodiments, the one or more supplemental specimens are obtained from the subject at a harvesting location in the tumor resection defect that is adjacent to an originating location of the respective tissue section in the tumor specimen.

Yet another aspect of the present disclosure provides an electronic device, comprising a display, an input device, one or more processors, memory, and one or more programs, where the one or more programs are stored in the memory and configured to be executed by the one or more processors, the one or more programs including instructions for performing any of the methods disclosed herein.

Still another aspect of the present disclosure provides a non-transitory computer-readable storage medium having stored thereon program code instructions that, when executed by a processor, cause the processor to perform any of the methods disclosed herein.

Yet another aspect of the present disclosure provides a system comprising, at a first electronic device, a display, an input device, one or more processors, memory, and one or more programs, where the one or more programs are stored in the memory and configured to be executed by the one or more processors, the one or more programs including instructions for a method for localizing close or positive margins in a tumor specimen from a subject. In some embodiments, the method comprises obtaining a first three-dimensional representation of the tumor specimen and a first plurality of annotations to the first three-dimensional representation of the tumor specimen; receiving, from a second electronic device, a first three-dimensional representation of a tumor resection defect in the subject that corresponds to the tumor specimen; displaying, on the display, a customizable user interface comprising, for each respective tissue section in a corresponding plurality of tissue sections from the tumor specimen, one or more user affordances for providing at least a corresponding margin status of the respective tissue section, where the displaying further comprises displaying the customizable user interface at the second electronic device, responsive to a first user interaction with respect to the customizable user interface at the first electronic device, the first user interaction is displayed on the customizable user interface at the second electronic device, and responsive to a second user interaction with respect to the customizable user interface at the second electronic device, the second user interaction is displayed on the customizable user interface at the first electronic device; receiving, from the second electronic device, a second plurality of annotations to the first three-dimensional representation of the tumor resection defect, where the second plurality of annotations comprises, for each respective tissue section in the corresponding plurality of tissue sections that satisfies a first margin status criterion based on the corresponding margin status, an indication of one or more supplemental specimens obtained from the tumor resection defect that map to the respective tissue section; and generating a report for the subject using, at least, the first three-dimensional representation of the tumor specimen, the first plurality of annotations, the first three-dimensional representation of the tumor resection defect, the second plurality of annotations, and the customizable user interface.

In some embodiments, the one or more programs further comprises instructions for forming a composite three-dimensional representation of the tumor specimen by modifying the first three-dimensional representation of the tumor specimen to include the first plurality of annotations, and communicating, to the second electronic device, one or more composite three-dimensional representation instructions for displaying an image overlay on the second electronic device comprising the composite three-dimensional representation in the form of the first plurality of annotations and the first three-dimensional representation of the tumor specimen, thereby producing a second three-dimensional representation of the tumor specimen.

In some embodiments, the receiving the first three-dimensional representation of a tumor resection defect further comprises displaying, on the display of the first electronic device, the first three-dimensional representation of the corresponding tumor resection defect.

In some embodiments, the first plurality of annotations comprises a first subset of annotations, each respective annotation in the first subset of annotations indicating an orientation of the first three-dimensional representation of the tumor specimen relative to the subject, a second subset of annotations, each respective annotation in the second subset of annotations indicating a respective tissue section in the corresponding plurality of tissue sections from the tumor specimen, and a third subset of annotations, each respective annotation in the third subset of annotations indicating the corresponding margin status for a respective tissue section in the plurality of tissue sections, wherein the corresponding margin status indicates whether the respective tissue section has a close or positive margin based upon a pathological analysis of the respective tissue section.

In some embodiments, the indication of one or more supplemental specimens obtained from the tumor resection defect that map to the respective tissue section comprises, for each respective supplemental specimen in the one or more supplemental specimens, a location and a breadth of the respective supplemental specimen relative to the first three-dimensional representation of the tumor resection defect.

In some embodiments, the one or more programs further include instructions for forming a composite three-dimensional representation of the tumor resection defect by modifying the first three-dimensional representation of the tumor resection defect to include the second plurality of annotations, and displaying an image overlay on the first electronic device comprising the composite three-dimensional representation in the form of the second plurality of annotations and the first three-dimensional representation of the tumor resection defect, thereby producing a second three-dimensional representation of the tumor resection defect.

In some embodiments, the first margin status criterion is satisfied when the respective tissue section has a close or positive margin based upon a pathological analysis of the respective tissue section.

In some embodiments, the one or more user affordances of the customizable user interface comprises, for each respective tissue section in the plurality of tissue sections, a first affordance for inputting a first annotation that indicates an identity of the tumor specimen, a second affordance for inputting a second annotation that indicates an identity of the respective tissue section, a third affordance for inputting a third annotation that provides the corresponding margin status for the respective tissue section, a fourth affordance for updating a review status of the respective tissue section from a first review status to a second review status, and a fifth affordance for appending the one or more supplemental specimens to the plurality of tissue sections.

Additional Example Embodiments for Localizing Close or Positive Margins in a Tumor Specimen from a Subject.

The present disclosure provides imaging tools that can be used to implement and test this new paradigm. In some embodiments, the systems and methods of the present disclosure advantageously provide a communication protocol utilizing 3D scans of tumor resection specimens and patient defects. In some implementations, the systems and methods of the present disclosure advantageously: (1) increase clarity and efficiency of communication between pathologist and surgeon; (2) reduce anesthesia duration; and (3) provide a more complete and transparent record of surgical treatment, including 3D modeling of surgical extent in the patient.

For example, in some implementations, the final output report will follow the patient to provide precise documentation of the surgery for radiation oncologists who may need to provide follow-up therapy. The presently disclosed pipeline comprises the hardware, software, tools, and standards that will be used for this communication between the OR and pathology lab. Head and neck cancer resection specimens are indeed the most anatomically complex, with multiple non-orthogonal margin planes. Advantageously, development of a protocol in this area can be translated to other types of resections. Patients with higher T stage cancers, or specimens from more anatomically complex regions are most likely to benefit from this protocol. Because the presently disclosed systems and methods do not change the manner in which specimens are sampled, or how cancer is detected, the protocol can be readily deployed.

The advent of rapid, low-cost 3D scanning technology can address the communication impasse of attempting to communicate complex, crucial three-dimensional information with mere words and 2-dimensional diagrams. 3D surface scanners can generate high-resolution scans (e.g., at least 0.1 mm³ 3D spatial resolution and/or at least 1.3 megapixel surface texture resolution) of resected tissue prior to pathologic dissection. In an example embodiment, a specimen scan is obtained as part of a preliminary investigation. A surgeon can be easily oriented to the specimen thanks to any maintained landmarks (e.g., mucosa, bone, and/or teeth). Each margin plane in the specimen can be extensively dissected and surveyed. The specimen scan can be further annotated with markings that correspond to the identity of histological sections that will be reported, where markings on the sample are placed digitally by the pathologist on the scanned image after dissection and generating results. This 3D model is then used in communication with the surgeon to establish gross anatomical positioning of the margins for further excision.

Even with the availability of 3D models, the process of surgeons scrubbing out of the OR, traveling to the pathology lab, then traveling back and scrubbing into the OR to resume surgery results in a burden that leads to additional confusion and frustration. The added travel time provides an opportunity for confusion or miscommunication to arise. Because the specimen scans are digitally acquired and annotated, they can be viewed remotely using telecommunications software. Thus, OR surgeons can discuss margin status with a pathologist in another room. Using this setup, the surgeons do not need to leave the OR, saving time and easing the communication burden.

### Additional Benefits

The present disclosure provides, in some embodiments: (1) the creation of high-resolution 3D models of tumor surgical resection specimens which can be easily labeled and annotated to provide a common communication tool between surgeons and pathologists; (2) creation of 3D models of the patient defect, which-if positive surgical margins are found-will be annotated with the desired additional resection locations; and (3) development of a coordinate-mapping system between 3D scanned resection specimen and patient surgical defect, allowing for visualization of the final surgical extent within the body. The primary impact of this work is to address the known communication gap between surgeons and pathologists regarding resection margin status using readily-available hardware tools, reduce anesthesia duration, improve outcomes, and provide a wealth of data for future study and reporting. As mentioned previously, because the actual manner in which specimens are sampled, or cancer is detected, is unchanged, the systems and methods disclosed herein can be readily deployed. Merging 3D resection specimen modeling with 3D tumor histology modeling has been reported by a group in Finland in the context of a postoperative instructional aid [11], and other groups have invested the use of augmented or virtual reality for image-guided surgery [12], but these technologies are in early developmental stages and are technically cumbersome. In contrast, the systems and methods of the present disclosure investigate 3D surface scanning for remote, precise, enhanced pathologist and surgeon communication that can be deployed immediately.

### A Scanning, Annotation, and Reporting Protocol for Tumor Resection Specimens and Patient Defects.

One aspect of the present disclosure provides a standardized protocol for specimen mapping, annotation, and reporting that may advantageously reduce anesthesia duration and increase OR utilization efficiency.

Generally, operating room times are significantly extended by the consultation between surgeon and pathologist. This consultation involves scrubbing out of the OR, travel time to the frozen lab, time spent discussing the specimen's margins, travelling back to and scrubbing back into the OR, and resumption of the surgery. The discussion itself is often made more difficult due to miscommunication between the pathologist and surgeon, and the dissociation of the surgical specimen from the patient's anatomy. A 3D scanning, annotation, and reporting protocol can overcome these communication gaps by: (1) simplifying the pathologist's reporting on anatomic margin planes, (2) obviating the need for the surgeon to leave the OR and discuss with the pathologist in person, thus eliminating the need to scrub out and back in, travel time, etc.; (3) clarifying locations of supplemental margins; and (4) producing a final document which visualizes extent of surgery and increases transparency to downstream healthcare providers.

An illustrative example of an experimental approach demonstrating this aspect of the present disclosure follows. During surgery, tumor resections can be scanned in the frozen section (FS) suite, for instance, by placing the tumor specimen onto a platform for scanning. In some embodiments, the platform rotates, thereby allowing the specimen to be scanned from a first angle to a second angle. See, for example, FIGS. 1A-B. Any number of scans can be taken, from any number of orientations. In some embodiments, the scanner is an Artec Space Spider 3D handheld scanner (Artec3D, Santa Clara, CA, USA) in the frozen section suite. This device is capable of scanning at 0.1 mm³ 3D spatial resolution, along with a surface texture resolution of 1.3 megapixels. In some embodiments, both the resected specimen and the patient defect in the OR suite can be scanned. The resulting specimen file scan can be preprocessed (e.g., automatically) to remove surface holes, remove self-intersecting faces, and/or compute the normal vectors for each triangular face. This can make the scan amenable to surface analysis and computation, as well as simplify the structure for annotation. Following preprocessing, any additional cleanup can be performed by the technician prior to saving the resulting mesh. All operations performed on the scan can be recorded and saved in a pipeline file for reproducibility.

In some implementations, the processed scan is loaded into a rendering and annotation application in accordance with some embodiments of the present disclosure, allowing the pathologist to view, manipulate, and annotate the sample. This software can enable technicians to load, annotate, save, and export their annotations and views. In contrast, currently available 3D annotating software is inadequate for even for these tasks. The annotations themselves can be formatted into a standardized coordinate system and export file format that is semantically and ontologically labeled.

In some embodiments, annotations comprise the following structures: (1) points, including a three-dimensional coordinate on the surface of the scan; (2) polygons, including a set of points that delineate a closed shape along the mesh surface, as well as a normal vector pointing in the direction of the outward face to define the orientation; (3) lines, including a set of points that are connected in sequence but do not necessarily create a closed shape (and do not have a normal vector to indicate orientation); and/or (4) text labels, including a set of text. Text labels can further be classed into “detached” labels with no including location, or they can be associated with another annotation (point, polygon, or line) describing a physical aspect of the scan. The text itself can be displayed separately from the mesh, with visual indicators to locations on the mesh surface. Other annotation structures are possible, as will be apparent to one skilled in the art. Alternatively or additionally, in some implementations, annotations comprise the set of structures to describe most aspects of margin status.

Shown in FIG. 2 is a close-up of a preliminary patient scan mesh (dark gray structure), with an example of a point annotation indicator (black arrow) and an associated text annotation (“Point Annotation”). The mesh is zoomed in to closely show the point annotation; the annotation software can be designed to rotate and move as the mesh is turned, so the objects are always indicating areas in relation to one another.

In preliminary testing, resection specimen scanning prior to margin assessment adds 10 to 15 minutes of delay to the time point where the margin results are generated. This does not disrupt current operating room workflows from the point of view of the surgeon. For instance, in some implementations, scan time is dependent on the model of the hardware, which is expected to improve with more powerful scanners. Additionally, annotations can be added to the 3D representation during production of H&E slides, such that it does not further interfere with pathology workflow. Moreover, communication between the pathology lab and operating room can take place over internal computer networks using existing teleconferencing software, in accordance with hospital IT protocols. In some embodiments, the length of time required to generate patient defect scans varies according to several factors (e.g., defect size, type of scanner, user technique, etc.), as will be apparent to one skilled in the art.

For surgeries using the scanned annotation and communication protocol, anesthesia duration and time spent communicating between surgeon and pathologist can be measured (e.g., from time of arrival of the tissue sample in the pathology lab to conclusion of surgery). These times can be compared with equivalent measurements taken during surgeries where the scanning, annotation, and virtual communication was not done. In general, average communication times are expected to be significantly reduced. Further, depending on the length of the overall surgery, overall OR occupation time is expected to be lower. Due to the lack of changes to the core workflow of cancer detection and margin assessment, no change in cancer recurrence rates are expected.

Nevertheless, because of the length and variability of typical head and neck surgical cases, statistically significant decreases in anesthesia duration may be difficult to observe. Additionally, “difficult” cases may take longer regardless of the use of the scanning protocol. In cases where this is observed, cases can be stratified based on surgical and pathological criteria such as anatomic sites and tumor stage to ensure that “average” times are being accurately compared.

a Point-to-Point Mapping Protocol Between the Resection and Defect Scans to Localize Regions of Concern within the Defect Scans and Provide a Full Surgical Extent Model.

Another aspect of the present disclosure provides digital point-to-point mapping between surgical specimen and patient defect that may advantageously lead to improved communication by precisely communicating margin locations on the patient's anatomy.

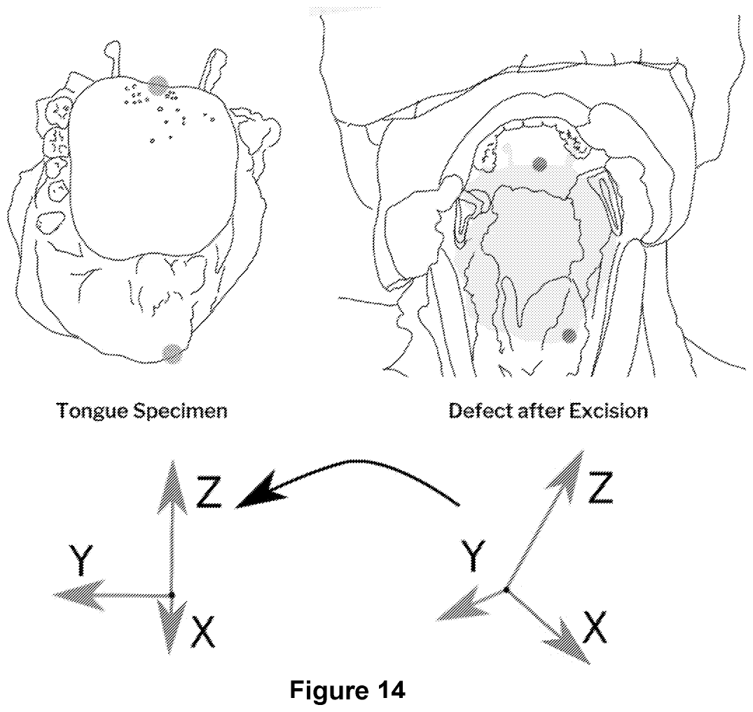

Generally, removal of the specimen from the patient results in a change of the coordinate space (e.g., translation, rotation, and shearing) resulting in a “specimen-space” and “patient-space” which are out of alignment (see, e.g., FIG. 14). Because of the dissociation between the two, communication of anatomical orientation is typically done manually by marking the specimen and describing the location relative to the patient. This change in space can be computationally compensated through a nonrigid transformation of the 3D specimen model to the “void” created by the patient defect.

As described above, a first aspect of the present disclosure focuses on the 3D scanning and annotation of the resection specimens and surgical defects. The present aspect connects these two anatomic landscapes by performing, in some implementations, a nonrigid registration between the two coordinate systems. Connecting the coordinate systems allows the pathologist to point to a margin plane, which can then also be highlighted in the defect scan. This demonstrates the exact anatomic locations within the patient and precisely directs the surgeon as to where supplemental margin harvesting is required. The success of this method can be measured in consultation between the surgeon and pathologist in describing margin locations.

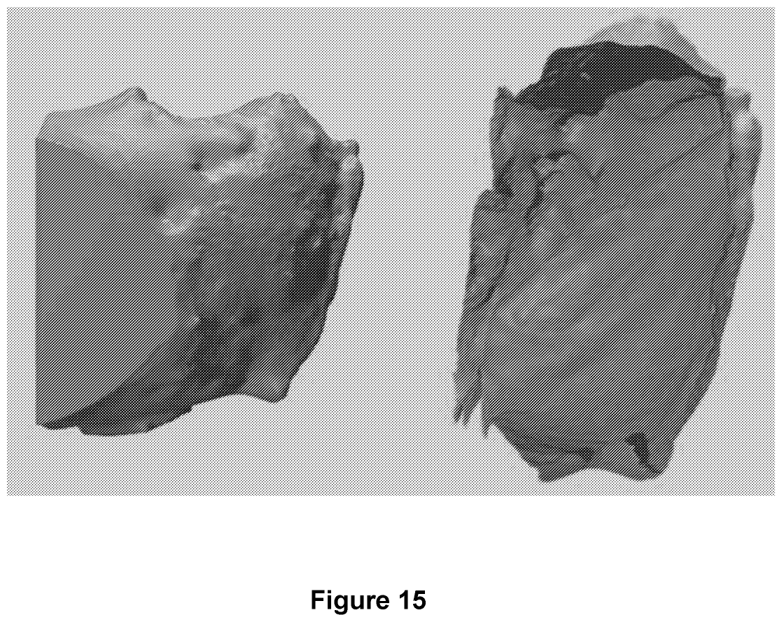

An illustrative example of an experimental approach demonstrating this aspect follows. Following surgical excision of the specimen, the patient defect is scanned in the OR suite using the same scanner hardware as the tumor specimen. The defect scan can include the exposed surgical site and the extent of the wound created from the excision. This file can be saved in the native format to the scanner and loaded into the scanning software outlined above. FIG. 15 shows an example of a specimen scan (left structure) and corresponding hypothetical patient defect scan (right structure); this defect scan was generated synthetically for demonstration by copying the specimen scan and distorting it. In practice, the differences between specimen and defect scans may differ (e.g., be larger), but with similar landmarks for registration.

In some embodiments, because of the deformations between specimen and defect, registration can be performed between the defect 3D model and the patient defect to bring the two coordinate spaces into alignment. This registration process can involve the following steps. In some implementations, the defect scan is preprocessed (e.g., automatically preprocessed) in a manner similar to the specimen scan above; specifically, all holes are removed, self-intersecting planes are resolved, and/or the surface normal vectors are computed. Next, in some embodiments, the surface normal vectors are inverted; this step can be used to produce a complementary surface on the defect that can be easily registered to the surface of the specimen that was removed. The mesh can be re-processed as before to ensure that the inversion has not introduced defects. Automated landmark detection can then identify surface features on the defect and the corresponding specimen scan to generate source and target meshes for registration. Rigid registration with iterative closest points (ICP) [13], [14] can be performed to align the two models to one another.

Following registration, a combined scene containing the specimen and the defect can be created that allows the pathologist to move, rotate, and annotate both the specimen model and the registered defect model. In some implementations, this scene has the ability to “snap” the specimen back to the defect, to show how the annotations are positioned relative to the patient anatomy.

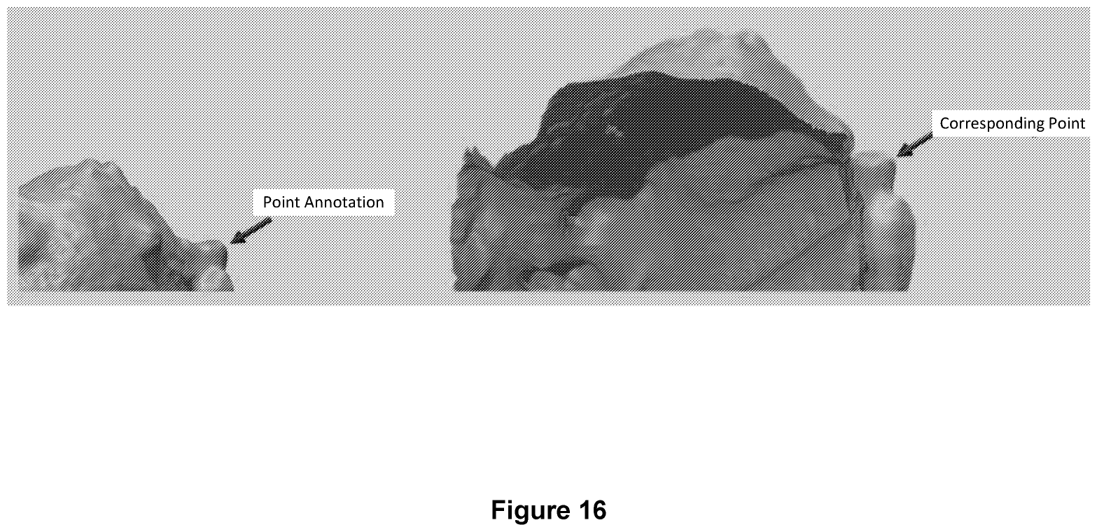

During videoconferencing, the pathologist can “drop a pin” or “paint a margin plane” on the specimen, and the corresponding region on the defect scan can be similarly highlighted and viewed by the surgeon in the OR. This representation can be updated in real time during videoconferencing. Shown in FIG. 16 is an illustrative demonstration of this correspondence mapping; shown are the two mesh models from FIG. 15 (left structure: specimen, right structure: defect), with a point annotation indicated on both the specimen and the defect (e.g., a margin of concern). This representation presents the pathologist's annotation in the anatomical context of the patient, allowing the surgeon to immediately see the affected area and perform additional harvesting in that area.

In some embodiments, the performance of the mapping protocol can be evaluated via discussions with pathologists and surgeons to determine whether the registration of the specimen and the defect produces a clearer communication tool compared with annotation of the specimen alone.

As described above, the mapping protocol can be used to register the specimen and the defect and provide a surgeon with a clear understanding of margin locations as they relate to the surgical defect, with little or no additional commentary from the pathologist required.

In some embodiments, landmark detection is performed automatically (e.g., using a landmark detection algorithm). Nevertheless, 3D model registration in accordance with the present disclosure may encounter various obstacles. For instance, if the landmark detection algorithm fails to find sufficient surface points for registration, manual annotation of landmarks can be used to aid in the registration approach. This can be performed by the pathologist during the annotation of the specimen during their analysis and communication. In some implementations, the ICP algorithm fails to find a mapping between landmarks on the two models. Without being limited to any one theory of operation, this may be due to problems with initialization of the registration and the parameters used to drive the registration approach. Both of these can be modified by the pathologist (e.g., with guidance from technicians). In some implementations, the process of “inverting” the defect to produce a registration target for the specimen model may not result in a good registration target; in some such implementations, more involved “puzzle” approaches that use spherical coordinate surface descriptors can be used to find matching surfaces and compute the registration between the two surfaces.

### REFERENCES

[1] A. Koivuholma et al., “Three-Dimensional Presentation of Tumor Histopathology: A Model Using Tongue Squamous Cell Carcinoma,” Diagnostics, vol. 11, no. 1, Art. no. 1, January 2021, doi: 10.3390/diagnostics11010109.
[2] H. H. Glas, J. Kraeima, P. M. A. van Ooijen, F. K. L. Spijkervet, L. Yu, and M. J. H. Witjes, “Augmented Reality Visualization for Image-Guided Surgery: A Validation Study Using a Three-Dimensional Printed Phantom,” J. Oral Maxillofac. Surg., vol. 79, no. 9, p. 1943.e1-1943.e10, September 2021, doi: 10.1016/j.joms.2021.04.001.
[3] Y. Chen and G. Medioni, “Object modelling by registration of multiple range images,” Image Vis. Comput., vol. 10, no. 3, pp. 145-155, April 1992, doi: 10.1016/0262-8856 (92) 90066-C.
[4] P. J. Besl and N. D. Mckay, “A method for registration of 3-D shapes,” IEEE Trans. Pattern Anal. Mach. Intell., vol. 14, no. 2, pp. 239-256, February 1992, doi: 10.1109/34.121791.
   Further Example Embodiments for Localizing Close or Positive Margins in a Tumor Specimen from a Subject.

### Frozen Section Analysis in Head and Neck Surgical Pathology

### Introduction

Surgery is the mainstay of treatment for head and neck cancer. Oncologically sound resections require complete removal of all malignancy. Frozen section represents the diagnostic gold standard for intraoperative pathologic assessment of surgical margins for malignancies. Otolaryngology is the surgical specialty which uses the highest volume of frozen sections; one academic center recorded 1,517 frozen sections submitted by otolaryngology over one year, representing 37% of all frozen sections performed during that interval.¹ Virtually all head and neck surgeons utilize frozen section in their surgical practice, most often for margin assessment.² Positive margins are strongly associated with locoregional recurrence and mortality, and become the basis for postoperative adjuvant therapy.^{3,4} While achieving tumor-free margins is of utmost importance to all head and neck surgeons, in practice, there are numerous debates and a lack of standardization for the role and method of intraoperative pathologic consultation.⁵

### Basic Protocol

In some embodiments, standard frozen section techniques generally comprises the following process:^{5,14-16}
1. The specimen is delivered from the operating room to the frozen laboratory.
2. Resection specimens submitted en bloc must first be oriented anatomically, which requires an appreciation of the three-dimensional (3D) aspects of the specimen and visible landmarks. Orientation can be facilitated by the surgeon placing a clip or suture to mark the specimen in a way that the pathologist understands the anatomic details of the resection.
3. Accuracy of specimen orientation can be further facilitated by direct face-to-face communication between the surgeon and pathologist to provide anatomic context. It is vital to communicate the true margin planes, as opposed to interface with an air-containing cavity, or a tear in the tissue.
4. Important intraoperative and radiographic information should also be conveyed in addition to regions of concern.
5. Supplemental margins taken from the surgical defect must also be precisely oriented so that the pathologist can understand where that tissue “fits” relative to the specimen.

Specimen orientation is a critical first step for accurate diagnosis. The level of experience of the pathologist, as well as intimate knowledge of head and neck anatomy, greatly impacts the degree of surgeon engagement needed during this initial stage. After orientation, the specimen is quickly grossed. Each surface plane may be inked in a different color, typically with skin, mucosa, and deep soft tissue margins being of greatest interest.¹⁷ Resection margins are visually surveyed by making multiple cuts, 5 to 10 mm apart, throughout all margin planes. Regions of concern are then selected for histologic examination. In some embodiments, bracketing or bookending the regions of concern with additional samples for histology is performed. Perpendicular sections are preferred, as they allow for measurements of margin distance. By contrast, parallel (shave) sections can be useful when the margin appears widely free of tumor.¹⁷ Tissue sections are placed on a tissue chuck, embedded in optimal cutting temperature (OCT) compound, and cooled down inside the cryostat. Five-micron sections are cut. The section is mounted on a glass slide, fixed, and stained, normally with hematoxylin and eosin. The slides are interpreted microscopically by the pathologist. Lastly, the results are communicated back to the operating room, typically by telephone or intercom. Without accurate and efficient communication, the entire process can be rendered meaningless. In some embodiments, difficult communication gaps arise with intraoperative margin mapping.

In general, intraoperative turnaround times for head and neck frozen sections average 19.7 minutes, with mean technical time 13.7 minutes and mean interpretive time of 6.0 minutes.¹⁸ These data are derived mostly from small specimens requiring minimal grossing. In some embodiments, complex en bloc specimens demand considerably longer turnaround time, sometimes upwards of 45 minutes for the entire intraoperative consultation. Not only does the above protocol vary among institutions, but also among individual pathologists and surgeons.¹⁴

Frozen section analysis has inherent limitations that can be classified as interpretive, sampling, technical, and communication.^{19,20} The freezing process can cause diminished cytologic fidelity; one expects the lesional cells to look different. This can lead to interpretive error. The inability to evaluate the entire continuous margin creates a sampling vulnerability, especially for large three-dimensionally complex head and neck specimens.¹⁶ Decalcification of bony specimens usually requires at least twenty-four hours, thus excluding bone margins from routine frozen section analysis.²¹ Adipose tissue is not amenable to frozen section. Sampling error, mislabeling, and miscommunication have been associated with inaccurate intraoperative diagnosis. Time pressure is another aspect of frozen section which makes it inherently vulnerable to error.²² Checklists and other quality improvement interventions have aimed to improve safety and enhance communication in frozen section.^{23,24} However, the frozen section process overall has not changed meaningfully for many decades.

### Definitions of a Positive Margin

Many authors have noted the lack of truly universal definitions for positive, clear (negative), or “close” head and neck surgical margins.^{5,25} A commonly accepted definition of a clear margin is a radial distance of at least 5 millimeters between the carcinoma and the resection edge on microscopic evaluation. The National Comprehensive Cancer Network (NCCN) Guidelines, Version 1.2022, states the following definitions: a clear margin is a distance of 5 mm or more from the invasive tumor front to the resected margin. Positive margin is defined as “cut-through” of either carcinoma-in-situ or frank invasive carcinoma. However, carcinoma-in-situ at margins may need to be accepted in the setting of diffuse multifocal carcinoma-in-situ. A close margin is defined as 2 to 5 mm between invasive tumor front to the resected margin, depending on the anatomic site involved.²⁶ (The NCCN guidelines make an exception for glottic cancers, for which 1-2 mm margins are deemed adequate.) Various other margin definitions throughout the literature were well-summarized by members of the International Head and Neck Scientific Group in 2013 and 2019, along with the prognostic implications of such diverse definitions as reported by their individual authors.^{5,14}

Surveys of the American Head and Neck Society (AHNS) membership were collected in 2005 and again in 2020 to solicit opinions on margin definitions and frozen section practices.^{2,25} Only 46% of the 2005 survey cohort² and 57% of the 2020 cohort²⁵ accepted the conventional 5 mm definition, suggesting considerable heterogeneity in margin definitions across institutions and individual surgeons. The absence of a universal standard positive margin definition creates difficulty in comparing oncologic outcomes in multicenter trials, especially because postoperative adjuvant therapy treatment decisions are often based on these definitions.

Tissue shrinkage ex vivo is another well-known phenomenon. Once a specimen has been removed from the body and released from muscular tension, it shrinks linearly by 25-75% depending on the tissue composition, according to various reports mainly in animal models.^{27,28} Therefore the 5 mm of histologic margin clearance measured on a microscope slide roughly corresponds to 1 cm of gross tissue within the operative field.²⁰

Margin Sampling: Defect-Driven Vs. Specimen-Driven

The two possible means of margin assessment are: sampling the tumor bed (defect-driven), or sampling the resection specimen ex vivo (specimen-driven) (Table 1). As discussed, head and neck frozen section margin practices are highly variable among North American and international centers.^{2,25,29} Defect-driven vs. specimen-driven margin strategies differ across institutions and among individual physicians. Although a true consensus has not yet been reached, a growing body of literature favors the specimen-driven margin approach, which has been linked to improved local control and oncologic outcomes (Table 2).^{2,30-42} The 2005 AHNS survey showed that 14% of responding surgeons preferred a specimen-driven, while 76% preferred to sample from the surgical bed.² In the follow-up survey fifteen years later, the portion of responding surgeons indicating a preference for specimen-driven margins rose to 55%.²⁵

The specimen-driven margin approach allows the pathologist to more reliably assess the full surface of the tumor-margin interface. This is more labor-intensive and usually requires a specialty expertise in head and neck pathology. Currently, Amit and colleagues⁴¹ have performed the only prospective randomized controlled trial comparing each approach in 71 consecutive oral cavity squamous cell carcinoma resections. Close/positive margins requiring harvesting of supplemental tissues were seen in 43% of the specimen-driven margin resections, compared to 10% in the defect-driven arm, suggesting that defect-driven frozen sections underestimate margin inadequacy. This is borne out in the final pathology negative margin rates of 84% and 55%, in the specimen- and defect-driven arms, respectively. One pitfall of the defect-driven margin approach is the tendency for surgeons to limit sampling to mucosa. In a vast majority of cases, positive margins occur at the deep soft tissue, rather than the more readily visible mucosa. In a study of 301 oral and oropharyngeal cancer resections, less than 2% resulted in inadequate margins based on mucosal margins alone.⁴³ It stands to reason that efforts to improve margin analysis therefore must focus on not only the mucosa, but also on the deep soft tissue planes. The three dimensional construct of head and neck oncologic surgery demands that all tissue planes must be evaluated and determined to be clear of residual disease. The choice of margin strategy still remains up to the surgeon.

### Challenges & Pitfalls in Head and Neck Surgical Pathology

Head and neck composite resections, and especially oral cavity cancers, are among the most challenging specimens for accurate pathologic margin determination. Tumors arising in the oropharynx, hypopharynx, and larynx also produce three-dimensionally complex resection specimens. These require the pathologist's careful attention to various resection planes in order to achieve correct and complete margin sampling. Such resection planes are not always obvious, and most general pathologists are unlikely to have a thorough understanding of the three-dimensional anatomy of the head and neck. Liberal resection of unnecessarily wide margins is not advisable as it can impact functional outcomes and quality of life. This principle is important in any solid tumor resection, but all the more so given the anatomic constraints of the head and neck region.

Biological factors further add to the complexity of head and neck surgical specimens and their intraoperative pathologic analysis.^{21,44} Recurrent disease will show changes of prior surgery and/or radiation that can distort anatomy and histology. Sialometaplasia with radiation atypia may mimic carcinoma. Field cancerization may result in multiple unexpected synchronous cancers in close proximity. To gauge tumor aggressiveness and counsel the surgeon appropriately, the worst pattern of invasion (WPOI) classification should also be considered during frozen section, e.g., small tumor islands separate from main tumor mass (WPOI-4), vs. dispersed tumor satellites (WPOI-5).^{5,45}

Bone margins represent a technical challenge as calcified bone requires decalcification. Well-accepted workarounds include frozen section analysis of cancellous bone scrapings; the smaller bony trabeculae can be cut on a cryostat. Sampling periosteal tissue and inferior alveolar nerve, although not perfect surrogates, may also reveal the state of the bone margin.^{46,47}

### Intraoperative Communication

Generally, clear communication between the surgical and pathology teams allows for effective and efficient frozen section analysis. Unfortunately, intraoperative communication remains a major challenge. The numerous handoffs between different personnel, and potential points of technical failure during the time-sensitive process, have been the subject of quality improvement initiatives.^{2,3,24}

Specimen-driven margin assessment, while superior in terms of local control, adds an additional burden for intraoperative communication. A simple binary diagnosis, i.e. positive vs. negative for carcinoma, suffices for tissue slivers harvested from the tumor bed and sampled en face. In contrast, much more data must be verbally transmitted to the operating room in the specimen-driven approach. The precise location of positive/close margins of concern needs to be communicated; vague terms like “deep” are insufficient. Simple specimen diagrams sketched by the pathologist prior to frozen section can help.^{21,48} Diagrams still do not obviate the awkwardness of verbally communicating the precise margin coordinates via telephone. Thus, surgeons commonly break scrub multiple times during complex resections, and repeatedly visit the frozen section laboratory for face-to-face consultation. In the authors' experience, this practice is necessary to achieve precise communication and gain actionable information for harvesting necessary supplemental margins. However, repeated trips by the surgeon, back and forth from the pathology lab to the operating room are time-consuming and inefficient. In addition, following margin analysis of the entire specimen, the integrity of that specimen is drastically compromised and no longer provides a point of reference for further discussions.

Even if face-to-face consultation enhances mutual understanding, it cannot fully solve the difficult problem of relocating and reconciling supplemental margins. The current NCCN Guidelines state: “If the surgeon obtains additional margins from the patient, the new margins should refer back to the geometric orientation of the resected tumor with a statement by the pathologist that this is the final margin of resection and its histologic status.”²⁶ This margin reconciliation process is performed at the discretion of the surgeon, but requires close cooperation with a head and neck pathologist. In reality, margin reconciliation is subject to significant ambiguity, as there is no basic method in widespread use for ensuring that the additional tissue resected from the tumor bed actually correlates to the area of margin positivity. It is known that initial positive/close margins, followed by negative supplemental margins, are associated with poorer local control and disease-specific survival than initial negative margins.⁴⁹ This underscores the importance of effective intraoperative communication for margin reconciliation.

### Postoperative Documentation of Margin Status

Postoperative documentation and communication of final margin status has received little attention in the literature. Downstream stakeholders involved in the care of the patient may need to patch together the operative and pathology reports to understand better the location of margins of concern, and their reconciliation with supplemental margins. The radiation oncologist is acutely concerned with final margin status in planning the precise fields of adjuvant therapy. Over the longer term, it is not uncommon for patients to relocate and seek further therapy or survivorship care with new physicians who are naïve to the details of past diagnosis and treatment. Accordingly, in some embodiments, it is important to produce a robust and detailed record of the intraoperative margin analysis. Operative reports and final pathology reports are essential documents that not only establish oncologic staging, but also provide substantial communication value for multidisciplinary treating physicians. In their fully text-based format, traditional pathology reports are unable to convey adequate information concerning the sites of margin positivity or closeness. Visuospatial representations of margin status, including frozen section specimen mapping and intraoperative diagnosis, are needed to ensure complete postoperative documentation and clinical communication.

### Innovations in Intraoperative Pathology & Surgical Navigation

A number of recent innovations have aimed to improve intraoperative margin assessment and surgical navigation in head and neck cancer.^{50,51} Some approaches serve as an adjunct to conventional frozen section analysis, while others play an experimental role essentially in lieu of histologic analysis (Table 3). Kain and colleagues have offered an excellent comprehensive review of innovative modalities to improve intraoperative tumor margin assessment.¹⁵ A complete account of the basic science principles underlying each of these approaches is beyond the scope of this review. Ultimately, every experimental strategy for improved intraoperative diagnostics, communication, and imaging modalities has its unique advantages and limitations.

### Introducing an Approach for 3D Specimen Mapping

The present disclosure provides, in some aspects, a virtual three-dimensional (3D) specimen mapping technique which enhances and optimizes intraoperative communication. 3D scanning technology is a mainstay in industrial manufacturing, quality control, and reverse engineering applications. Recent advancements have drastically reduced the cost, operational complexity, and physical size of 3D scanning equipment. This technology is now entering the healthcare realm and anatomic pathology in particular,⁵² with a recent case report demonstrating the feasibility of 3D scanning of a fresh glossectomy specimen prior to formalin fixation.⁵³ Using a structured light desktop 3D scanner (Einscan-SP, Shining 3D), Hangzhou, China), the ability to reliably capture the 3D surface topography of a fresh surgical specimen received in the frozen section laboratory has been demonstrated, producing a digital 3D specimen representation. Image acquisition time is consistently less than twelve minutes. The scan data is processed and imported into a graphics workspace (Paint 3D, Microsoft, Redmond, WA) as a 3D object file.

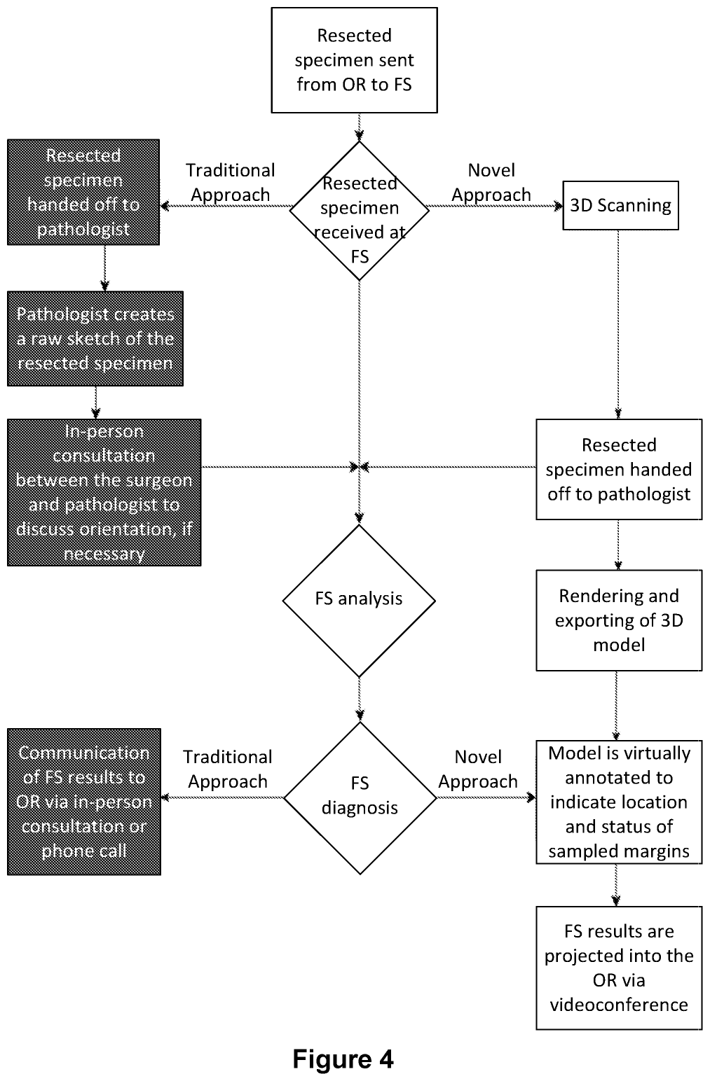

After 3D scanning, the frozen section process is carried out as described above. The virtual 3D representation then becomes the vehicle by which to communicate the results. The pathologist directly annotates the 3D specimen representation to illustrate regions of concern. The 3D representation is displayed by video-conferencing on overhead monitors in the OR; this becomes the reference point for discussion, rather than the inked, dissected resection specimen, now unrecognizable to the surgeon, or the crude specimen sketch. The surgeon no longer needs to break scrub and all stake holders are “on the same page” (see, for example, FIGS. 3-4). In addition, this 3D scan of the specimen serves as a permanent reference point in the patient's electronic medical record.

### Specimen-Defect Mapping

In some embodiments, the present disclosure addresses the critical issue of positive/close margin reconciliation, e.g., translating specimen-driven margin findings back to the tumor bed for optimal supplemental resection. In some implementations, 3D scanning proves useful to capture the patient's ablative defect and allow for correlation with the 3D specimen map, potentially guiding immediate margin revision. In some embodiments, real-time audiovisual communication allows for a qualitatively enhanced intraoperative discussion of surgical margins, and reduction of time needed for in-person communication.

In some embodiments, virtual 3D specimen maps also improve the quality of medical documentation for downstream health care providers. For instance, in some embodiments, visuospatial documentation of the tumor margins, at the specimen and theoretically within the surgical defect, serve as a long-term resource for radiation oncologists, future cancer care teams, or survivorship care at another institution. This technique has the potential to become standard for comprehensive head and neck oncologic care. Indeed, the 3D specimen maps generated intraoperatively have begun to facilitate discussions of adjuvant radiotherapy at a multidisciplinary head and neck tumor board. This allows the care team to visualize and therefore improve their understanding of the surgical intervention.

### REFERENCES

1 McIntosh E R, Harada S, Drwiega J, Brandwein-Gensler M S, Gordetsky J. Frozen section: guiding the hands of surgeons? Ann Diagn Pathol. 2015; 19 (5): 326-329. doi: 10.1016/j.anndiagpath.2015.07.004
2. Meier J D, Oliver D A, Varvares M A. Surgical margin determination in head and neck oncology: current clinical practice. The results of an International American Head and Neck Society Member Survey. Head Neck. 2005; 27 (11): 952-958. doi: 10.1002/hed.20269
3 Looser K G, Shah J P, Strong E W. The significance of “positive” margins in surgically resected epidermoid carcinomas. Head & Neck Surgery. 1978; 1 (2): 107-111. doi: 10.1002/hed.2890010203
4 Loree T R, Strong E W. Significance of positive margins in oral cavity squamous carcinoma. The American Journal of Surgery. 1990; 160 (4): 410-414. doi: 10.1016/S0002-9610 (05) 80555-0
5. Hinni M L, Ferlito A, Brandwein-Gensler M S, et al. Surgical margins in head and neck cancer: a contemporary review. Head Neck. 2013; 35 (9): 1362-1370. doi: 10.1002/hed.23110
6. Wright J R. The Development of the Frozen Section Technique, the Evolution of Surgical Biopsy, and the Origins of Surgical Pathology. Bulletin of the History of Medicine. 1985; 59 (3): 295-326.
7. Taylor T. A New Freezing Microtome. Proceedings of the American Society of Microscopists. 1882; 4:153-155. doi: 10.2307/3220523
8. Gal A A, Cagle P T. The 100-Year Anniversary of the Description of the Frozen Section Procedure. JAMA. 2005; 294 (24): 3135-3137. doi: 10.1001/jama.294.24.3135
9. Cullen T S. A rapid method of making permanent specimens from frozen sections by the use of formalin. Johns Hopkins Hospital Bulletin. 1895; (6): 67.
10. Rutkow I. The Rise of Modern Surgery: An Overview. In: Sabiston Textbook of Surgery. 21st ed. Elsevier; 2022:2-19. Accessed Nov. 15, 2021. https://www-clinicalkey-com.proxy.library.vanderbilt.edu/#!/content/book/3-s2.0-B9780323640626000013?scrollTo=%23h10000261
11. Wilson L B. A Method for the Rapid Preparation of Fresh Tissues for the Microscope. Journal of the American Medical Association. 1905; XLV (23): 1737. doi: 10.1001/jama.1905.52510230037003c
12. Bloodgood J C. Biopsy in Diagnosis of Malignancy. Southern Medical Journal. 1927; (1927; 18-28).
13. Singer J A. Joseph Colt Bloodgood: pioneer in surgical pathology. Curr Surg. 2001; 58 (6): 593-594. doi: 10.1016/s0149-7944 (01) 00534-7
14. Thomas Robbins K, Triantafyllou A, Suárez C, et al. Surgical margins in head and neck cancer: Intra- and postoperative considerations. Auris Nasus Larynx. 2019; 46 (1): 10-17. doi: 10.1016/j.anl.2018.08.011
15. Kain J J, Birkeland A C, Udayakumar N, et al. Surgical margins in oral cavity squamous cell carcinoma: Current practices and future directions. Laryngoscope. 2020; 130 (1): 128-138. doi: 10.1002/lary.27943
16. Bullock S, Balzer B, Marchevsky A. Guide to the Preparation of Frozen Sections-ClinicalKey. In: Intraoperative Consultation: Foundations in Diagnostic Pathology.; 2015. Accessed Nov. 15, 2021. https://www-clinicalkey-com.proxy.library.vanderbilt.edu/#!/content/book/3-s2.0-B9781455748235000032?scrollTo=%23h10000245
17. Westra W H, Phelps T H, Hruban R H, Isacson C. Complex Specimens. In: Westra W H, Phelps T H, Hruban R H, Isacson C, eds. Surgical Pathology Dissection: An Illustrated Guide. Springer; 2003:44-47. doi: 10.1007/0-387-21747-9_8
18. Laakman J M, Chen S J, Lake K S, et al. Frozen Section Quality Assurance: Using Separate Frozen Section Slide Preparation Times and Interpretative Time Measurements to Improve Process. Am J Clin Pathol. 2021; 156 (3): 461-470. doi: 10.1093/ajcp/aqaa259
19. Gandour-Edwards R F, Donald P J, Wiese D A. Accuracy of intraoperative frozen section diagnosis in head and neck surgery: experience at a university medical center. Head Neck. 1993; 15 (1): 33-38. doi: 10.1002/hed.2880150108
20. Wenig B M. Intraoperative consultation (IOC) in mucosal lesions of the upper aerodigestive tract. Head Neck Pathol. 2008; 2 (2): 131-144. doi: 10.1007/s12105-008-0053-6
21. Weinstock Y E, Alava I, Dierks E J. Pitfalls in determining head and neck surgical margins. Oral Maxillofac Surg Clin North Am. 2014; 26 (2): 151-162. doi: 10.1016/j.coms.2014.01.003
22. Volmar K E, Idowu M O, Souers R J, Karcher D S, Nakhleh R E. Turnaround time for large or complex specimens in surgical pathology: a College of American Pathologists Q-Probes study of 56 institutions. Arch Pathol Lab Med. 2015; 139 (2): 171-177. doi: 10.5858/arpa.2013-0671-CP
23. Sams S B, Wisell J A. Discordance Between Intraoperative Consultation by Frozen Section and Final Diagnosis. Int J Surg Pathol. 2017; 25 (1): 41-50. doi: 10.1177/1066896916662152
24. Chen Y, Anderson K R, Xu J, Goldsmith J D, Heher Y K. Frozen-Section Checklist Implementation Improves Quality and Patient Safety. Am J Clin Pathol. 2019; 151 (6): 607-612. doi: 10.1093/ajcp/aqz009
25. Bulbul M G, Zenga J, Tarabichi O, et al. Margin Practices in Oral Cavity Cancer Resections: Survey of American Head and Neck Society Members. Laryngoscope. Published online Aug. 22, 2020. doi: 10.1002/lary.28976
26. NCCN Guidelines Version 1.2022. Head and Neck Cancers. National Comprehensive Cancer Network (NCCN). Published Dec. 8, 2021. Accessed Dec. 26, 2021. https://www.nccn.org/guidelines/guidelines-detail
27 Mistry R C, Qureshi S S, Kumaran C. Post-resection mucosal margin shrinkage in oral cancer: Quantification and significance. Journal of Surgical Oncology. 2005; 91 (2): 131-133. doi: 10.1002/jso.20285
28. Cheng A, Cox D, Schmidt B L. Oral Squamous Cell Carcinoma Margin Discrepancy After Resection and Pathologic Processing. Journal of Oral and Maxillofacial Surgery. 2008; 66 (3): 523-529. doi: 10.1016/j.joms.2007.08.040
29. Li M M, Puram S V, Silverman D A, Old M O, Rocco J W, Kang S Y. Margin Analysis in Head and Neck Cancer: State of the Art and Future Directions. Ann Surg Oncol. 2019; 26 (12): 4070-4080. doi: 10.1245/s10434-019-07645-9
30. Maxwell J H, Thompson LDR, Brandwein-Gensler M S, et al. Early Oral Tongue Squamous Cell Carcinoma Sampling of Margins from Tumor Bed and Worse Local Control. JAMA Otolaryngol Head Neck Surg. 2015; 141 (12): 1104-1110. doi: 10.1001/jamaoto.2015.1351
31. Varvares M A, Poti S, Kenyon B, Christopher K, Walker R J. Surgical margins and primary site resection in achieving local control in oral cancer resections. The Laryngoscope. 2015; 125 (10): 2298-2307. doi: 10.1002/lary.25397
32. Varvares M A, Walker R J, Chiosea S. Does a specimen-based margin analysis of early tongue cancer better predict local control? Laryngoscope. 2016; 126 (11): 2426-2427. doi: 10.1002/lary.26081
33. Buchakjian M R, Tasche K K, Robinson R A, Pagedar N A, Sperry S M. Association of Main Specimen and Tumor Bed Margin Status with Local Recurrence and Survival in Oral Cancer Surgery. JAMA Otolaryngol Head Neck Surg. 2016; 142 (12): 1191-1198. doi: 10.1001/jamaoto.2016.2329
34. Du E, Ow T J, Lo Y T, et al. Refining the utility and role of Frozen section in head and neck squamous cell carcinoma resection. Laryngoscope. 2016; 126 (8): 1768-1775. doi: 10.1002/lary.25899
35. Horwich P, MacKay C, Bullock M, et al. Specimen oriented intraoperative margin assessment in oral cavity and oropharyngeal squamous cell carcinoma. J Otolaryngol Head Neck Surg. 2021; 50 (1): 37. doi: 10.1186/s40463-021-00501-5
36. Aaboubout Y, Ten Hove I, Smits RWH, Hardillo J A, Puppels G J, Koljenovic S. Specimen-driven intraoperative assessment of resection margins should be standard of care for oral cancer patients. Oral Dis. 2021; 27 (1): 111-116. doi: 10.1111/odi.13619
37. Prabhu A V, Sturgis C D, Lai C, et al. Improving margin revision: Characterization of tumor bed margins in early oral tongue cancer. Oral Oncol. 2017; 75:184-188. doi: 10.1016/j.oraloncology.2017.10.013
38. Kubik M W, Sridharan S, Varvares M A, et al. Intraoperative Margin Assessment in Head and Neck Cancer: A Case of Misuse and Abuse? Head Neck Pathol. 2020; 14 (2): 291-302. doi: 10.1007/s12105-019-01121-2
39. Barroso E M, Aaboubout Y, van der Sar L C, et al. Performance of Intraoperative Assessment of Resection Margins in Oral Cancer Surgery: A Review of Literature. Front Oncol. 2021; 11:628297. doi: 10.3389/fonc.2021.628297
40. Buchakjian M R, Ginader T, Tasche K K, Pagedar N A, Smith B J, Sperry S M. Independent Predictors of Prognosis Based on Oral Cavity Squamous Cell Carcinoma Surgical Margins. Otolaryngol Head Neck Surg. 2018; 159 (4): 675-682. doi: 10.1177/0194599818773070
41. Amit M, Na'ara S, Leider-Trejo L, et al. Improving the rate of negative margins after surgery for oral cavity squamous cell carcinoma: A prospective randomized controlled study. Head & Neck. 2016; 38 (S1): E1803-E1809. doi: 10.1002/hed.24320
42. Chang AMV, Kim S W, Duvvuri U, et al. Early squamous cell carcinoma of the oral tongue: Comparing margins obtained from the glossectomy specimen to margins from the tumor bed. Oral Oncology. 2013; 49 (11): 1077-1082. doi: 10.1016/j.oraloncology.2013.07.013
43. Woolgar J A, Triantafyllou A. A histopathological appraisal of surgical margins in oral and oropharyngeal cancer resection specimens. Oral Oncol. 2005; 41 (10): 1034-1043. doi: 10.1016/j.oraloncology.2005.06.008
44. Smits RWH, Koljenović S, Hardillo J A, et al. Resection margins in oral cancer surgery: Room for improvement. Head Neck. 2016; 38 Suppl 1: E2197-2203. doi: 10.1002/hed.24075
45. Brandwein-Gensler M, Smith R V, Wang B, et al. Validation of the histologic risk model in a new cohort of patients with head and neck squamous cell carcinoma. Am J Surg Pathol. 2010; 34 (5): 676-688. doi: 10.1097/PAS.0b013e3181d95c37
46. Bilodeau E A, Chiosea S. Oral squamous cell carcinoma with mandibular bone invasion: intraoperative evaluation of bone margins by routine frozen section. Head Neck Pathol. 2011; 5 (3): 216-220. doi: 10.1007/s12105-011-0264-0
47. Nieberler M, Stimmer H, Rasthofer D, Nentwig K, Weirich G, Wolff K D. Defining secure surgical bone margins in head and neck squamous cell carcinomas: The diagnostic impact of intraoperative cytological assessment of bone resection margins compared with preoperative imaging. Oral Oncol. 2020; 102:104579. doi: 10.1016/j.oraloncology.2020.104579
48. Brandwein-Weber M S. Textbook of Head and Neck Pathology. Springer International Publishing; 2018. doi: 10.1007/978-3-319-76106-0
49. Guillemaud J P, Patel R S, Goldstein D P, Higgins K M, Enepekides D J. Prognostic impact of intraoperative microscopic cut-through on frozen section in oral cavity squamous cell carcinoma. J Otolaryngol Head Neck Surg. 2010; 39 (4): 370-377.
50. Rosenthal E L, Warram J M, Bland K I, Zinn K R. The status of contemporary image-guided modalities in oncologic surgery. Ann Surg. 2015; 261 (1): 46-55. doi: 10.1097/SLA.0000000000000622
51. Singhal S. The Future of Surgical Oncology: Image-Guided Cancer Surgery. JAMA Surgery. 2016; 151 (2): 184-185. doi: 10.1001/jamasurg.2015.3604
52. Farahani N, Braun A, Jutt D, et al. Three-dimensional Imaging and Scanning: Current and Future Applications for Pathology. J Pathol Inform. 2017; 8. doi: 10.4103/jpi.jpi_32_17
53. Koivuholma A, Aro K, Mäkitie A, et al. Three-Dimensional Presentation of Tumor Histopathology: A Model Using Tongue Squamous Cell Carcinoma. Diagnostics (Basel). 2021; 11 (1). doi: 10.3390/diagnostics11010109
54. Williams M D. Determining Adequate Margins in Head and Neck Cancers: Practice and Continued Challenges. Curr Oncol Rep. 2016; 18 (9): 54. doi: 10.1007/s11912-016-0540-y
55. Kerawala C J, Ong T K. Relocating the site of frozen sections—is there room for improvement? Head Neck. 2001; 23 (3): 230-232. doi: 10.1002/1097-0347(200103)23:3<230::aid-hed1023>3.0.co;2-v
56. Black C, Marotti J, Zarovnaya E, Paydarfar J. Critical evaluation of frozen section margins in head and neck cancer resections. Cancer. 2006; 107 (12): 2792-2800. doi: 10.1002/cncr.22347
57. Lanschot CGF van, Mast H, Hardillo J A, et al. Relocation of inadequate resection margins in the wound bed during oral cavity oncological surgery: A feasibility study. Head & Neck. 2019; 41 (7): 2159-2166. doi: https://doi.org/10.1002/hed.25690
58. Aaboubout Y, Barroso E M, Algoe M, et al. Intraoperative Assessment of Resection Margins in Oral Cavity Cancer: This is the Way. J Vis Exp. 2021; (171). doi: 10.3791/62446
59. Westra W H, Sidransky D. Fluorescence Visualization in Oral Neoplasia: Shedding Light on an Old Problem. Clin Cancer Res. 2006; 12 (22): 6594-6597. doi: 10.1158/1078-0432.CCR-06-2253
60. Zhang R R, Schroeder A B, Grudzinski J J, et al. Beyond the margins: real-time detection of cancer using targeted fluorophores. Nat Rev Clin Oncol. 2017; 14 (6): 347-364. doi: 10.1038/nrclinonc.2016.212
61. Teraphongphom N, Kong C S, Warram J M, Rosenthal E L. Specimen mapping in head and neck cancer using fluorescence imaging. Laryngoscope Investig Otolaryngol. 2017; 2 (6): 447-452. doi: 10.1002/lio2.84
62. Lauwerends L J, van Driel PBAA, Baatenburg de Jong R J, et al. Real-time fluorescence imaging in intraoperative decision making for cancer surgery. Lancet Oncol. 2021; 22 (5): e186-e195. doi: 10.1016/S1470-2045 (20) 30600-8
63. Lee Y J, Krishnan G, Nishio N, et al. Intraoperative Fluorescence-Guided Surgery in Head and Neck Squamous Cell Carcinoma. Laryngoscope. 2021; 131 (3): 529-534. doi: 10.1002/lary.28822
64. Rosenthal E L, Warram J M, de Boer E, et al. Successful Translation of Fluorescence Navigation During Oncologic Surgery: A Consensus Report. J Nucl Med.
2016; 57 (1): 144-150. doi: 10.2967/jnumed.115.158915 65. Levy L L, Vila P M, Park R W, et al. High-Resolution Optical Imaging of Benign and Malignant Mucosa in the Upper Aerodigestive Tract: An Atlas for Image-Guided Surgery. ISRN Minim Invasive Surg. 2012; 2012. doi: 10.5402/2012/364285
66. van Keulen S, Nishio N, Fakurnejad S, et al. Intraoperative Tumor Assessment Using Real-Time Molecular Imaging in Head and Neck Cancer Patients. J Am Coll Surg.
2019; 229 (6): 560-567.e1. doi: 10.1016/j.jamcollsurg.2019.09.007
67. Stepan K O, Li M M, Kang S Y, Puram S V. Molecular margins in head and neck cancer: Current techniques and future directions. Oral Oncol. 2020; 110:104893. doi: 10.1016/j.oraloncology.2020.104893
68. Kennedy G T, Azari F S, Bernstein E, et al. Targeted Intraoperative Molecular Imaging for Localizing Nonpalpable Tumors and Quantifying Resection Margin Distances. JAMA Surgery. Published online Aug. 25, 2021. doi: 10.1001/jamasurg.2021.3757
69. Roberts N, Magee D, Song Y, et al. Toward routine use of 3D histopathology as a research tool. Am J Pathol. 2012; 180 (5): 1835-1842. doi: 10.1016/j.ajpath.2012.01.033
70. Pallua J D, Brunner A, Zelger B, Schirmer M, Haybaeck J. The future of pathology is digital. Pathol Res Pract. 2020; 216 (9): 153040. doi: 10.1016/j.prp.2020.153040
71. Barroso E M, Ten Hove I, Bakker Schut T C, et al. Raman spectroscopy for assessment of bone resection margins in mandibulectomy for oral cavity squamous cell carcinoma. Eur J Cancer. 2018; 92:77-87. doi: 10.1016/j.ejca.2018.01.068
72 Zhang J, Rector J, Lin J Q, et al. Nondestructive tissue analysis for ex vivo and in vivo cancer diagnosis using a handheld mass spectrometry system. Sci Transl Med. 2017; 9(406):eaan3968. doi: 10.1126/scitranslmed.aan3968
73. Pu J J, Choi W S, Yu P, Wong MCM, Lo A W I, Su Y X. Do predetermined surgical margins compromise oncological safety in computer-assisted head and neck reconstruction? Oral Oncol. 2020; 111:104914. doi: 10.1016/j.oraloncology.2020.104914
74 Essig H, Rana M, Meyer A, et al. Virtual 3D tumor marking-exact intraoperative coordinate mapping improve post-operative radiotherapy. Radiat Oncol. 2011; 6:159. doi: 10.1186/1748-717X-6-159
75. Bulbul M G, Tarabichi O, Parikh A S, et al. The utility of intra-oral ultrasound in improving deep margin clearance of oral tongue cancer resections. Oral Oncology. 2021; 122:105512. doi: 10.1016/j.oraloncology.2021.105512

TABLE 1
Summary of specimen-driven vs. defect-driven
frozen section margin analysis strategies.

| | Specimen-driven | Defect-driven |
|---|---|---|
| Advantages | Radial margin distance can be | Easier for guiding immediate |
| | assessed intraoperatively^{25, 34} | revision of initially positive |
| | Opportunity for better | margins²⁵ |
| | orientation of the tumor for the | Opportunity for clear labeling of |
| | pathologist²⁵ | margins by surgeon based on site of |
| | Surgeon can indicate which | excision |
| | areas on the specimen are of | Requires minimal-to-no |
| | most concern³⁴ | understanding of anatomy by the |
| | Improved local control and | pathologist³⁷ |
| | prognostic value^{30, 31, 32} | Avoids disruption of the main |
| | Statistically significant | specimen's permanent analysis²⁵ |
| | decrease in final positive | |
| | margin rate, which leads to a | |
| | cost reduction benefit based on | |
| | decreased re-resection time | |
| | and indicated follow-up | |
| | treatment³⁴ | |
| | May benefit the planning of | |
| | radiation treatment fields by | |
| | providing a better | |
| | documentation of spatial | |
| | relationship amongst tumor | |
| | margins⁴⁰ | |
| Limitations | Sampling error can occur with | Cannot identify radial margin |
| | large frozen section | distance intraoperatively^{30, 34, 40} |
| | specimens^{2, 34} | Potential for non-representative |
| | Potential deleterious effect on | sampling of tissue from tumor bed |
| | permanent section analysis by | and underestimate of positive |
| | diminishing the orientation and | margins |
| | integrity of the specimen³⁵ | Narrower margin clearance³⁰ |
| | Requires more resources; | Higher rate of positive final |
| | dedicated team of head and | margins^{30, 33} |
| | neck specialists³⁴ | Difficult to establish relationships |
| | Requires transmission of | amongst tissue fragments³⁷ |
| | complex 3D information for | Analysis of the deep margin may |
| | positive or close margins to | not be possible at certain subsites |
| | guide immediate supplemental | adjacent to neurovascular |
| | re-resection⁵⁴ | structures²⁹ |
| | | Associated with worse local |
| | | control³⁰ |
| | | Potentially no prognostic value with |
| | | regards to local recurrence-free |
| | | survival^{31, 32, 40} |

TABLE 2
Oncologic outcomes of specimen-driven (SD) vs. defect-driven
(DD) frozen section margin analysis in selected literature.

| First Author | | | Description of | |
|---|---|---|---|---|
| (Year) | Study Design | N | Sample | Findings |
| Maxwell et | Multicenter | 280 | pT1-T2N0 oral tongue | SD permanent margin status |
| al. (2015)³⁰ | retrospective | | SCC treated with | significantly correlated with |
| | cohort study | | primary surgery | LR-free survival |
| | | | | No prognostic value of DD |
| | | | | frozen margin status with |
| | | | | regards to LR-free survival |
| Amit et al. | Prospective | 71 | pT1-T4 oral cavity | Permanent margin positivity rate |
| (2016)⁴¹ | randomized | | SCC treated with | 45% in DD arm, 16% SD arm |
| | controlled | | primary surgery | Adjuvant therapy received in 8% |
| | trial | | | SD arm vs. 35% DD arm |
| Horwich et | Combined | 227 | Retrospective: pT1-T2 | Significant decrease in permanent |
| al. (2021)³⁵ | retrospective | | oral cavity SCC | margin positivity rate from |
| | & prospective | | Prospective: pT1-T4 | 12.9% to 0.9% after switching |
| | cohort study | | oral cavity and | from DD to SD protocol |
| | | | oropharyngeal SCC | |
| | | | All treated with | |
| | | | primary surgery | |
| Buchakjian | Retrospective | 406 | pT1-T4 oral cavity | DD frozen sections showed false- |
| et al. | cohort study | | SCC treated with | negative rate of 45% compared |
| (2016)³³ | | | primary surgery | to permanent SD margins |
| | | | | SD permanent margins |
| | | | | significantly associated with |
| | | | | LR, while DD frozen margins |
| | | | | were not significantly |
| | | | | associated with LR |
| Varvares et | Retrospective | 108 | pT1-T4 oral cavity and | Local recurrence rate 25% in DD, |
| al. (2015)³¹ | cohort study | | non-HPV | 17.6% in SD margins |
| | | | oropharyngeal | Assessing margins from the |
| | | | malignancy | resection specimen rather than |
| | | | Inclusion regardless of | the tumor bed consistently |
| | | | histology and prior | predicts local control |
| | | | treatment status | |
| Chang et al. | Retrospective | 126 | pT1-T2N0 oral tongue | No correlation between DD |
| (2013)⁴² | cohort study | | SCC treated with | margin status and LR |
| | | | primary surgery | Positive SD margin conferred a |
| | | | | relative risk of 2.5 (95% CI |
| | | | | 1-6.3) for LR |

LR, local recurrence.
SCC, squamous cell carcinoma.
CI, confidence interval.
All were single-institution studies unless otherwise noted.

TABLE 3
Summary of techniques for real-time intraoperative margin assessment.

| | | | | Location |
|---|---|---|---|---|
| | | | | of |
| Modality | Description | Strengths | Limitations | Analysis |
| Analog Pairwise | Numbered tags are placed | Reliable | Time- | Pathology |
| Tagging^{34, 55-58} | on both sides of the | relocation of | consuming | Lab |
| | resection line in a pair- | inadequate | Impedes | |
| | wise manner to promote | margins | surgical | |
| | anatomic orientation | | workflow | |
| | during specimen-driven | | | |
| | frozen section analysis | | | |
| Fluorescent | Administering fluorescently | Tissue-specific | Requires | Operating |
| Dyes and | labeled antibodies as an | Visual | specialty | Room |
| Autofluorescent | image-guided technique | feedback | spectrometers | |
| Imaging^{50, 59-64} | for outlining cutaneous and | intraoperatively | Not always | |
| | mucosal tumors in vivo | Repeated | tumor-specific | |
| | and ex vivo | imaging at | Useful | |
| | | each step of | compounds | |
| | | analysis | variable, | |
| | | | unstandardized | |
| Optical | High-resolution | Non-invasion | Keratinized | Operating |
| Coherence | microendoscope (HRME) | delineation | tissue can | Room |
| Tomography⁶⁵ | equipped with a fiberoptic | between | obscure nuclei | |
| | probe is paired with tissue | benign and | visualization | |
| | contrast agents to image | neoplastic | Tumors with | |
| | nuclei and cellular | epithelium | above-average | |
| | architecture | | nuclei density | |
| | | | are difficult to | |
| | | | distinguish | |
| Targeted | Anti-epidermal growth favor | In situ surgical | Image quality | Operating |
| Intraoperative | receptor (EGFR) antibody | imaging of | susceptible to | Room |
| Molecular | panitumumab conjugated | the primary | external light, | |
| Imaging⁶⁶⁻⁶⁸ | to near infrared dye allows | tumor | surface | |
| | for intraoperative tumor | Tumor-specific | reflection, and | |
| | image acquisition across | | camera | |
| | three different light | | positioning | |
| | settings | | Imaging results | |
| | | | vary | |
| | | | depending on | |
| | | | software | |
| | | | availability | |
| Digital | Integrated application of | Potential cost | Currently | Pathology |
| Pathology^{65, 69, 70} | information technology, | savings | lacking a | Lab + |
| | including digital images of | Promotes | standardized | Operating |
| | micro-/macroscopic | larger, | data | Room |
| | preparations, clinical data, | interconnected | infrastructure | |
| | and artificial intelligence, | information | | |
| | to refine the | systems | | |
| | communication of | | | |
| | information within a | | | |
| | collaborative infrastructure | | | |
| Raman | Utilizes the reflected | Intraoperative | Improper bone | Pathology |
| Spectroscopy⁷¹ | scattering of light from | bone margin | cuts can | Lab |
| | molecules within | assessment | decrease the | |
| | cancerous tissue to | Non- | quality of the | |
| | elucidate the molecular | destructive | Raman signal | |
| | composition and chemical | No reagents | Requires | |
| | components, thus | required | specialized | |
| | demarcating tumor from | | equipment | |
| | health tissue | | | |
| Handheld Mass | Ex vivo molecular analysis | Automated | Broad usage | Pathology |
| Spectrometry⁷² | of cancerous tissue using a | Biocompatible | requires | Lab |
| | disposable droplet | Rapid | assembling | |
| | dispenser that | Non- | and validating | |
| | characterizes cancer | destructive | databases for | |
| | biomarkers using mass | | tissue | |
| | spectrometry | | characterization | |
| Virtual Surgical | Computer-assisted | Improved | Time and | Preoperative + |
| Planning for | preoperative planning | surgeon- | resource- | Operating |
| Predetermined | (CAPP) outlines the | pathologist | intensive | Room |
| Margins^{73, 74} | macroscopic tumor size | and surgeon- | Specialized | |
| | and margins by overlaying | radiotherapist | equipment and | |
| | CT, MRI, and 3D imaging | interface | software | |
| | Margins can be marked | | required | |
| | virtually and stored for | | | |
| | future review | | | |
| Intra-Oral | Dynamic ultrasound | Whole-lesion | Can vary in | Operating |
| Ultrasound⁷⁵ | imaging performed at the | visualization | time- | Room |
| | time of surgery to evaluate | Measures depth | intensiveness | |
| | the 3-dimensional extent of | of invasion | Tumor | |
| | the tumor | Evaluate deep | extending into | |
| | | margin | the mandible is | |
| | | continually | inaccessible | |
| | | Widely | for evaluation | |
| | | available | Challenging to | |
| | | | visualize deep | |
| | | | margins in | |
| | | | bulky/ T4 | |
| | | | disease | |

### EXAMPLES

### Example 1—Utilizing 3D Head and Neck Specimen Scanning for Intraoperative Margin Discussions

### 1. Introduction

Surgical margin status is an invaluable tool when treating head and neck cancer. It is critical to establish a sufficient margin of healthy, uninvolved tissue (i.e., negative margins) surrounding the resected tumor. Surgical margins can be analyzed through either a defect-driven or specimen-driven approach. The former involves sampling sites from the tumor bed for analysis, while the latter investigates samples from select slices of the main specimen ex vivo. Though both techniques have their proponents, en bloc sampling in the specimen-driven approach is gaining favor.¹⁻³ Regardless of which method is used, the majority of head and neck surgeons rely heavily on frozen section results to assess the real time status of their excisional margins.

Unfortunately, the current method of communicating the margin results is outdated and inefficient; the process has remained stagnant for the past several decades.⁴ Commonly, the surgeon and pathologist must convene in-person to correctly orient the specimen relative to the surgical defect and convey margin results. All positive or close margins require that supplemental margins be taken from the precise corresponding location within the surgical defect. This task can be particularly difficult when considering the anatomical and three-dimensional (3D) complexity of commonly resected head and neck specimens. As a visual aid, pathologists often create crude, hand-drawn two-dimensional (2D) specimen maps to demonstrate the location of the sampled slices and the distance of the margins from the tumor. These drawings are susceptible to human error and misinterpretation, and they are rarely used for future reference and post-operative care management.

Physicians have expressed a clear desire for improved intraoperative image guidance to visualize resected tumors and the surrounding margins. This requires a solution that expedites the specimen-driven approach, is easily replicable, and improves the quality of communication between the surgeon and pathologist.⁵ Previous studies have attempted to improve histopathological analysis using various 3D modeling technologies. One method involves constructing a 3D model by serially layering 2D histological slides taken from the specimen⁶⁻¹⁰. Another method similarly produces a model using 2D preoperative imaging.¹¹⁻¹⁵ Neither technique is clinically applicable to frozen section analysis, however, because they use sophisticated modeling software that is time-intensive and requires highly trained specialists to operate.¹⁶

Optical 3D scanning is an emerging imaging modality that can be performed easily and much more rapidly. It has already become a mainstay in industrial and engineering fields, as well as certain medical specialties such as prosthetic development. There is one reported case of this technology being used for histopathological tumor analysis, in which a 3D model of squamous cell carcinoma of the tongue was generated to support permanent margin assessment postoperatively.¹⁶ A method was proposed in which optical 3D scanning technology is used intraoperatively to improve surgical oncology and frozen section analysis. Prior to slicing the specimen, an interactive digital model of the intact resection specimen is rendered and virtually annotated to indicate the exact location and status of all margins taken by the pathologist. These models are then projected directly into the operating room in via real-time video teleconference.

In the traditional frozen section approach, it is not uncommon that the surgeon is asked to return to the frozen section lab to review close or positive margins, only to find that the orientation of the specimen is virtually impossible to achieve due to its mutilation by the pathologic processing. One of the tremendous values of the proposed technique is that it produces an enduring image of the specimen that would otherwise be unrecognizable following the performance of frozen sections.

In this example, a qualitative review is presented of 55 head and neck surgical specimens that were used to generate 3D models and communicate margin results intraoperatively. Goals included demonstrating the feasibility of an improved frozen section workflow that incorporates a 3D imaging modality and to systematically assess the 3D image quality of common tumor specimens resected during head and neck surgery. Currently, this study is the first of its kind to report the use of 3D optical imaging to communicate surgical margin statuses intraoperatively.

### 2. Materials and Methods

All study experiments were carried out at the Mount Sinai West Department of Pathology in the frozen section laboratory. The specimens were taken from the following anatomical locations: facial skin, larynx, oral cavity, oropharynx, mandible, palatomaxillary, and parotid regions. The 3D surface topography of the specimens was imaged using a structured light desktop 3D scanner (Einscan-SP, Shining 3D), Hangzhou, China). This hardware is accompanied with an associated ExScan S software to render the virtual model (see, e.g., FIG. 1A-B). The model was then downloaded as a 3D object file and exported to a 3D modeling application (Paint 3D), Microsoft, Redmond, WA). Paint 3D was used to virtually “ink” the pathologist's sample locations on the specimen and annotate margin results. Paint 3D is free, easy to use, and compatible with the files exported from ExScan S. Lastly, Zoom was used as a video teleconferencing platform to facilitate the remote consultation between the pathologist in the frozen section laboratory and the surgical team in the operating room. This study was performed with the approval of the Mount Sinai Quality Assurance Committee.

### 2.1. Workflow

The specimen was delivered to the frozen section laboratory, where the pathologist examined and oriented the specimen and appreciates the regions of interest. Anatomically complex specimens were readily oriented with either clips or sutures placed by the surgeon at the time of the resection. The intact specimen was then scanned, and an initial virtual model was generated. The scan took an average of eight minutes to complete. Table 5 outlines the average time required to fully render, annotate, and generate a final virtual specimen model.

After scanning, the specimen was handed back to the pathologist for inking, mapping, sampling, grossing, and microscopic analysis. While the pathologist conducted the remaining standard procedure of frozen sectioning⁴, a technician refined the initial 3D model using the ExScan post-processing features. Once the status of the surgical margins were finalized, the locations of the sections examined were drawn directly onto the surface of the digital 3D model. The annotated specimen model was then projected onto overhead monitors in the operating room, allowing the pathologist and surgeon to visualize the results via video teleconference and discuss the specific sites that may require additional supplemental resections.

### 2.2. Scanning and Annotation Techniques

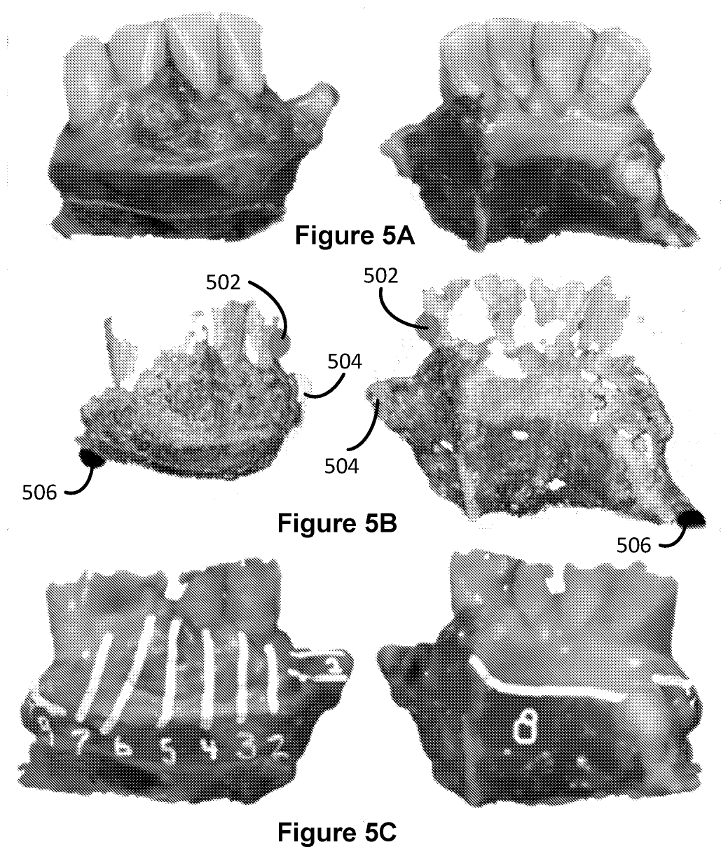

Optical scans were achieved by taking two 360-degree scans of the specimen, each from a different orientation. These renderings were then aligned using landmarks that are visible on both scans. During alignment, the person operating the scanner (the “operator”) considered two principal factors: the section of the specimen from which margins will be taken must be clearly visible, and the regions of the specimen that are not visible in the first orientation should be noted and made visible in the second orientation (FIG. 5A). The operator manually aligned the two scans with the ExScan software by selecting three pairs of identical landmarks shared between the two models (FIG. 5B). The scans were then virtually annotated in Paint 3D. The locations at which the pathologist sampled slices of the specimen were drawn and labeled directly onto the 3D models (FIG. 5C).

### 3. Results

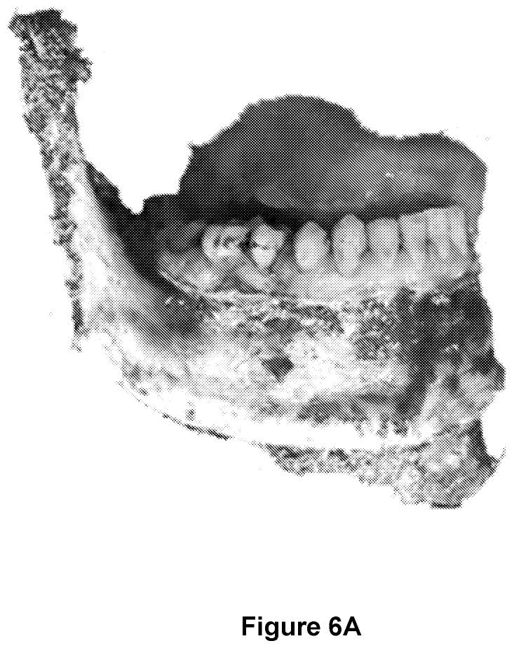

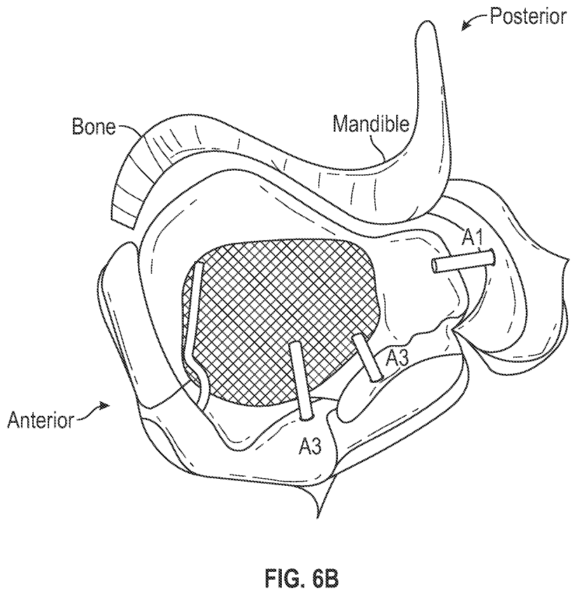

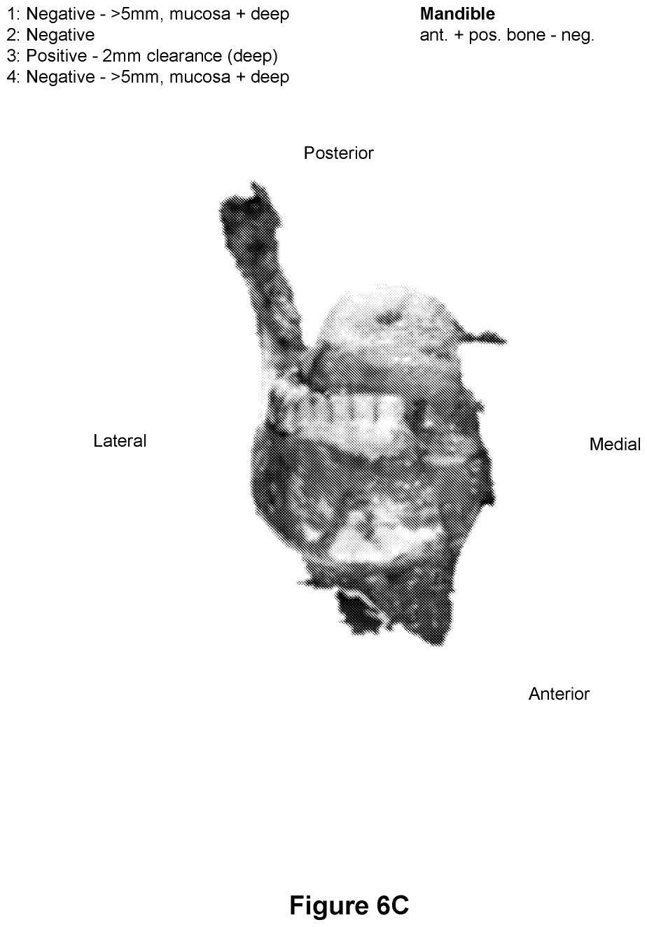

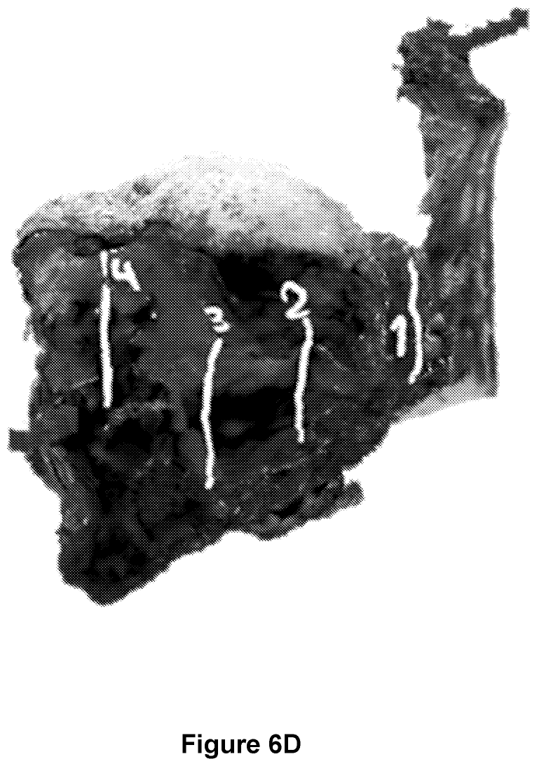

A total of 55 3D specimen models were produced. A summary of the specimens received from various anatomic subsites can be found in Table 4. The minimum size threshold beyond which specimens could not be accurately scanned was 3.0×3.0×3.0 cm. FIGS. 6-13 demonstrate representative images of the annotated specimen models from these sites. The total average time needed to scan the specimen, render, and edit the model while the standard margin analysis was performed, and annotate and communicate final margin results was 34 minutes (Table 5).

### 4. Discussion

### 4.1 Feasibility of Workflow

The abovementioned 3D scanning workflow was minimally disruptive and easily integrated into the standard steps of frozen sectioning. Although an initial 8 minutes on average are required to scan the gross specimen before handing it off to the pathologist, the operator can perform the rest of the modeling and annotation while the pathologist completes the typical frozen section procedure.

A large single-institution study reported that the current average turnaround time for delivering intraoperative head and neck frozen section results is 19.7 minutes.¹⁷ However, a majority of the frozen section procedures used to produce these findings were conducted on small specimens that required minimal grossing. Additionally, this data does not account for the time added when the surgeon must break scrub to consult with the pathologist directly in the frozen section laboratory, a process that varies considerably among institutions. Moreover, larger, more complex specimens can take significantly longer to assess, often approaching 40 minutes or more. Thus, the average 34 minutes needed for intraoperative frozen section results when using 3D scanning is likely comparable. Based on laboratory experience and perception of the value added, the added time is deemed to be justified when considering that the virtual model significantly improves the precision of communicated results and obviates the need for an even more time consuming in-person consultation.

### 4.2. Specimen Size

Larger specimens were typically rendered with a higher image quality. The largest specimens scanned were those resected from glossectomy, laryngectomy, and mandibulectomy procedures (FIGS. 6A-D). These specimens normally exceeded 8 centimeters along their largest dimension and provided surface areas of up to 40 square centimeters or more along their broadest side. The increased surface area enables the scanner to generate a larger point cloud with which it can construct the three-dimensional texture of the object and clearly demarcate the gross borders of bone, fat, skin, and mucosa.

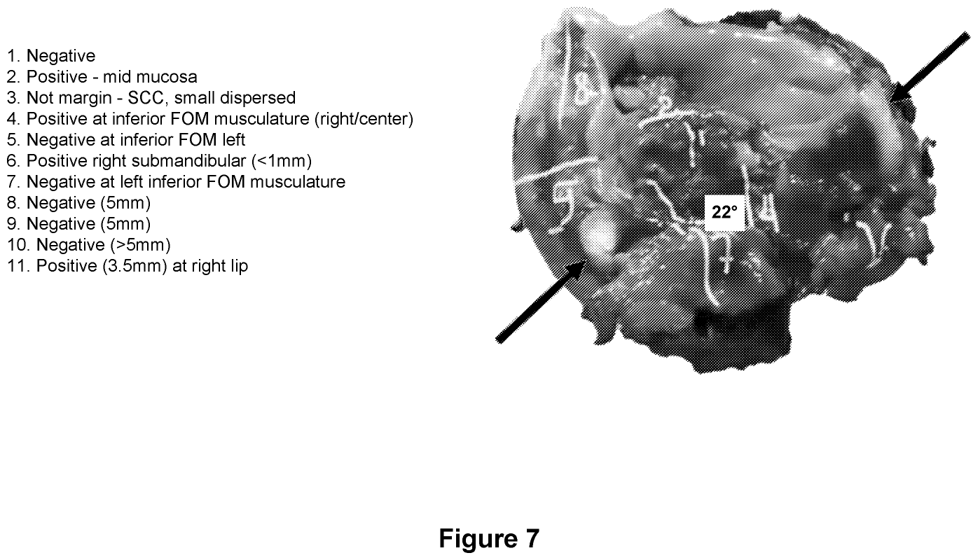

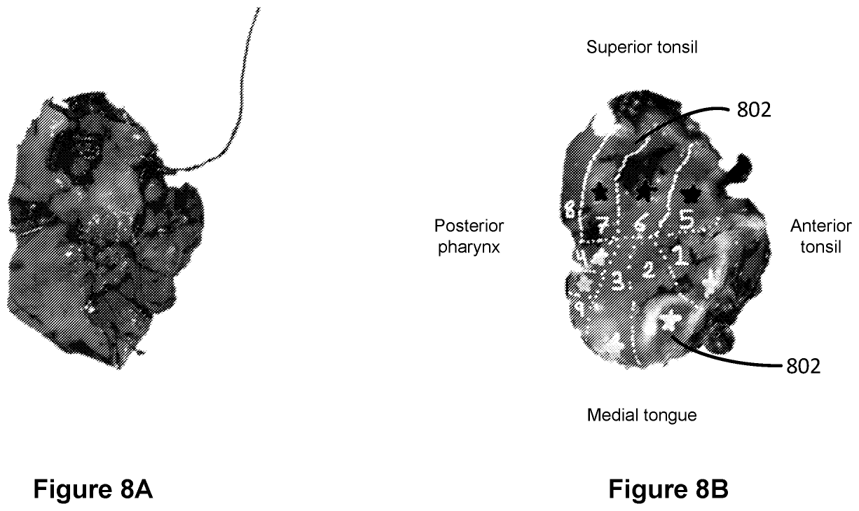

The ability to rotate the specimen in space is extremely helpful in orienting the surgeon during videoconferencing. Anatomic landmarks such as bone or teeth also contribute to visual orientation and serve as reference points. When desired, the pathologist can accentuate the borders of bone and/or mucosal tissue using the paintbrush tool within Microsoft Paint 3D (FIG. 7). It is obvious that annotating and rotating a 3D representation allows transmission of information far superior to even the best two-dimensional drawing. The minimum specimen size required to produce a viable scan is 3.0×3.0×3.0 cm. All of the tongue, laryngeal, and mandibulectomy specimens scanned at the study institution exceeded this minimum size threshold. Smaller specimens that approached the minimum size threshold included buccal resections and radical tonsillectomies (FIG. 8A-B). These resections provided more sparsely detailed texture models when compared to larger specimens. Still, their size did not impact the ability to orient the specimen and identify margins.

### 4.3. Specimen Color Contrast

Many specimens provide an inherent contrast in color between different anatomic subsites. This contrast is a critical feature that impacts the pathologist's ability to delineate mucosal and submucosal boundaries, specifically. Identifying and visually demonstrating these boundaries for the surgeon is particularly important when analyzing deep surgical margins. In en bloc resection, peripheral mucosal margins may be palpated and even observed grossly, but deeper margins are typically much more difficult to assess intraoperatively. Therefore, the surgeon relies on the pathologist to elucidate information regarding deeper margins, especially when considering features such as lymphovascular invasion (LVI) and perineural invasion (PNI) that are associated with deeper tumor infiltration.¹⁸

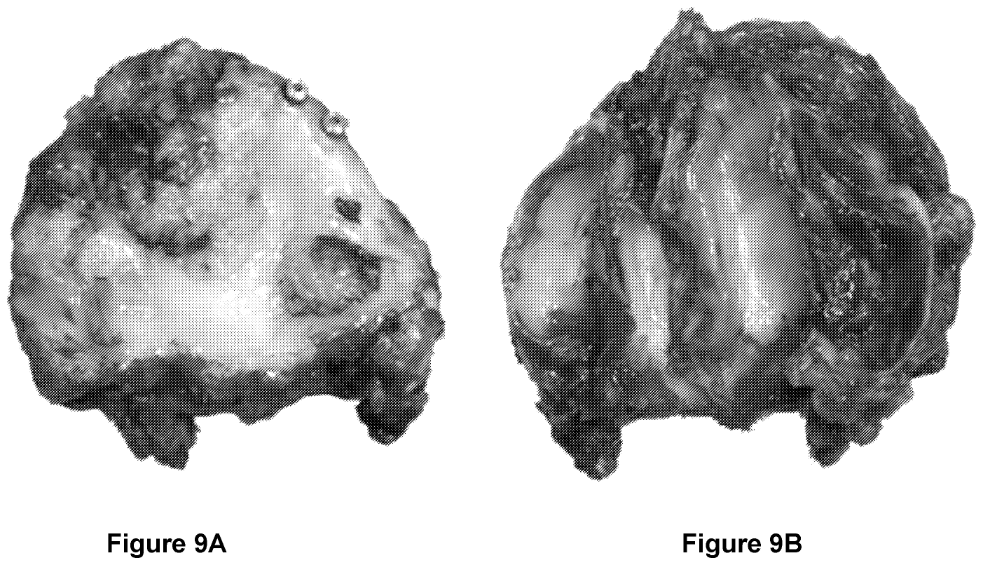

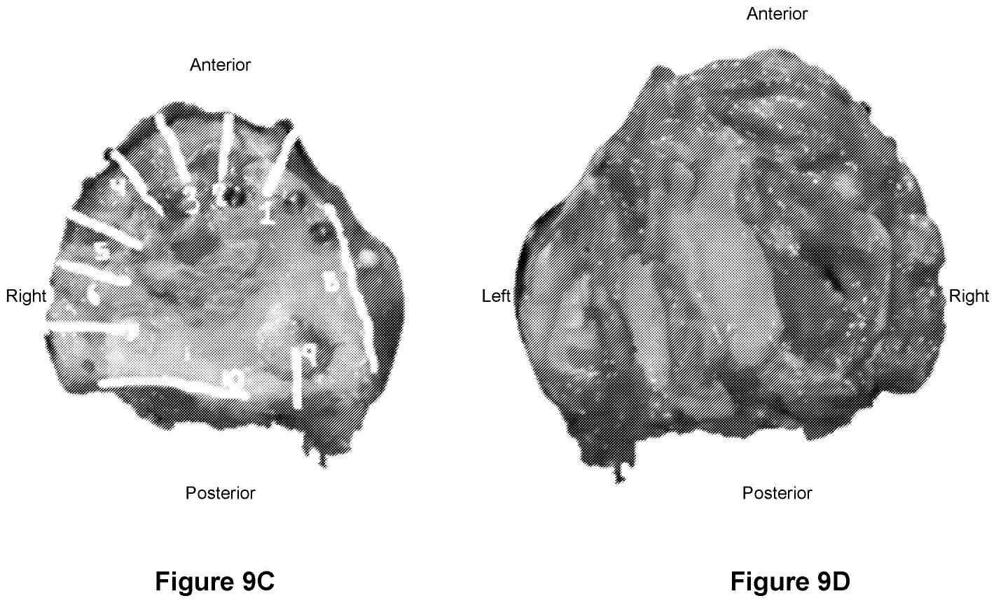

The technology used to generate three-dimensional renderings of resected tumor specimens utilizes structured-light scanning, a method that lends itself well to reflecting any slight contrasts in color between the superficial and deeper layers of human tissue. Certain specimens contain inherent contrasting features that are amenable to structured light optical scanning. For example, the innate coloration of buccal specimens provides the contrast needed to discern the boundaries of gingiva, skin, and outer and inner lip. Furthermore, laryngeal resections include the epiglottis and vestibule, which are light in color compared to the thyroid gland overlying the ventral surface. The scanner is also sensitive to the contrast in color between the mucosal surfaces of the endolarynx and trachea. Mandibular and maxillary specimens have visible bone that appears distinct from the adjacent mucosa and muscle that are additionally resected en bloc (FIGS. 9A-D). As a result of this sensitivity, the pathologist can precisely communicate the extent of the tumor to the surgeon in real-time.

Meanwhile, some mucosal surfaces in other specimens, namely glossectomy specimens, are darker in color. Dark regions pose an initial challenge to the structured light mechanics of the scanner. The deeper layers of the tongue, beneath the epithelial layer, along the surgeon's resection margin, are often dark. This can diminish the quality of the sagittal view of the model. This issue was more frequently encountered when scanning partial glossectomies. Still, even in cases where visibility was poorer, the clarity of margin analysis was not impacted because the lesion of interest was located on the brighter surfaces of the lateral tongue. When needed, adjusting the scanner's brightness level to its maximum setting improved the visibility and contrast in darkly colored regions. Thoroughly rinsing the specimen of any extraneous blood and fluid before patting it dry can also improve image quality.

### 4.4. Topographical Complexity

The geometric and surface-level complexity of head and neck specimens varies significantly. It was found that the EinScan SP is capable of generating accurate virtual three-dimensional models of nearly any specimen regardless of its shape and topography, given that it exceeds the minimum size threshold, and that the operator takes care to expose the specimen's most pertinent aspects to the camera.

Buccal and tonsil resections were consistently among the simplest in terms of their shape and surface. They were also consistently the smallest specimens scanned. However, their flat, circular shape still enabled the scanner to generate sophisticated surface renderings. Additionally, the smooth ovular shape of the tongue was easily rendered into a virtual model with high detail. In composite resections with segmental mandibulectomy, the continuous, rigid contour of the mandible provided a broad surface that enhanced the ability to accurately image the attached soft tissue.

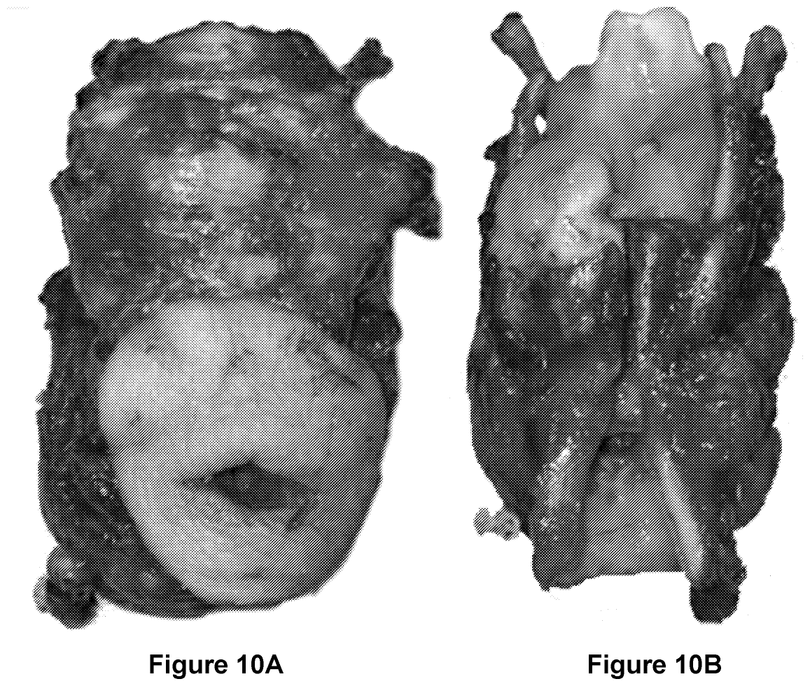

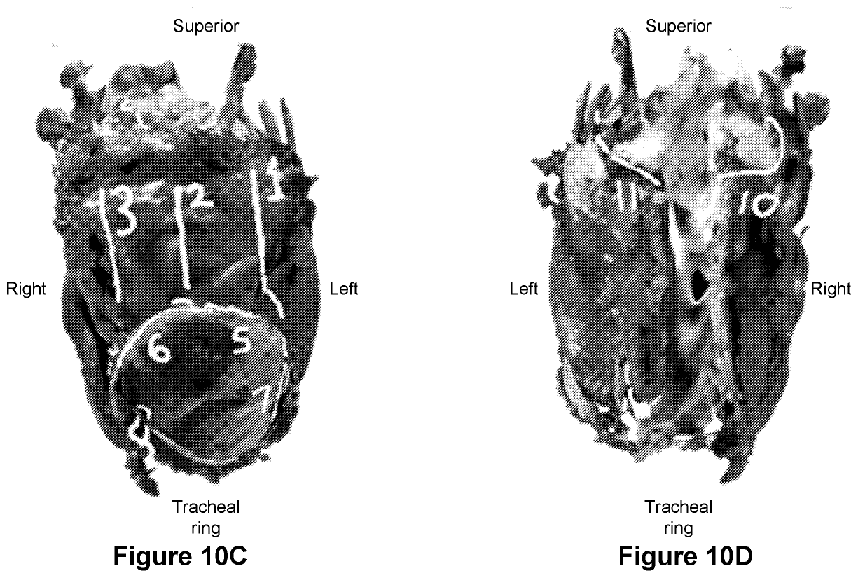

Other specimens required more preparation in order to optimally display the regions of interest to the scanner. The larynx is an excellent example in which the internal mucosal surface is difficult to visualize from the exterior. To gain visualization of this region, the dorsal larynx can be divided at midline and splayed open during the scan (FIGS. 10A-D).

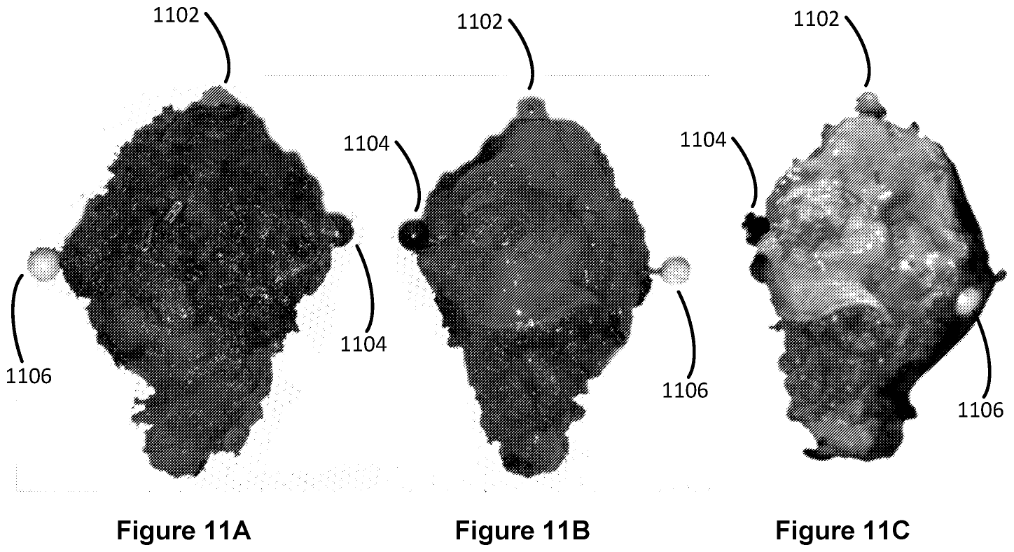

When scanning a specimen with complex structure and surface-level texture, it was found to be challenging to precisely interpret its anatomic orientation relative to the surgical defect. For example, resections taken from the posterior palate and oropharynx were texturally complex, yet often lacked any descriptive features that would help clarify how the specimen related to the patient's surrounding anatomy. As a solution, colored pins were inserted in the specimen to serve as an external fiduciary marking system (FIGS. 8A-B, FIGS. 11A-C). The scanner was able to register these pins with variable success. It is already common practice for surgeons to orient the specimen for the pathologist by placing clips or sutures and including a key that indicates how these markings relate to the defect. Placing fiduciary pins at these surgical markings, when successful, helped further designate anatomic direction and other key areas of interest on the scan. They also ensured that the surgeon and pathologist were aligned in their understanding of how the specimen correlated to the patient's surrounding anatomy. Additionally, using colored fiducials significantly increased the ease with which the virtual model was aligned and annotated during the post-processing phase.

### 4.5. Distinct Anatomic Landmarks

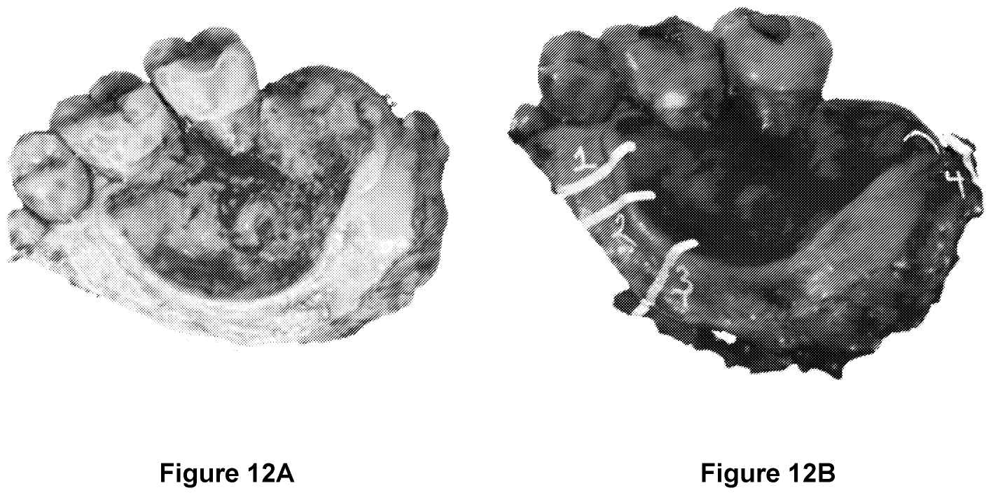

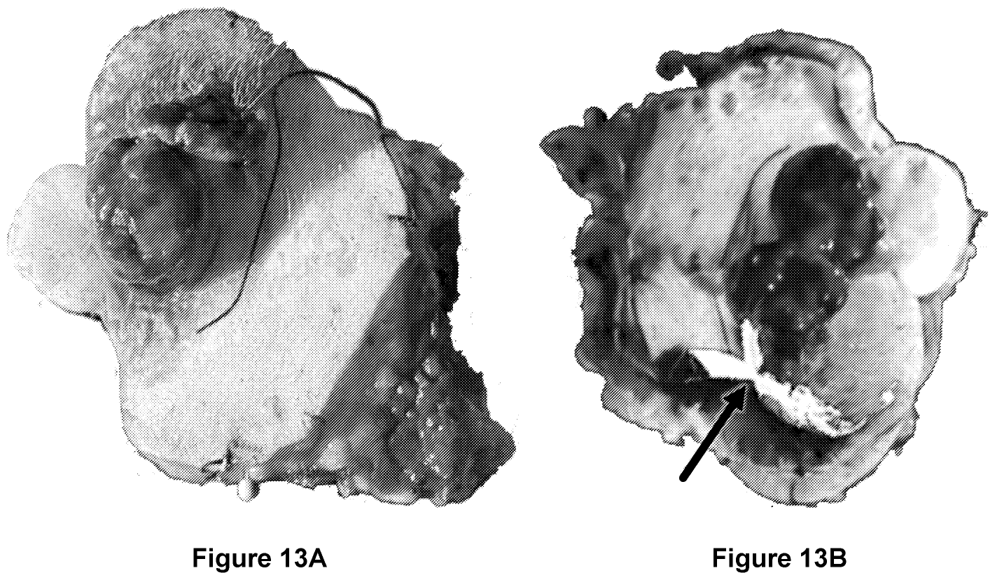

Depending on the location, some head and neck specimens included local anatomical features such as bone, teeth, and lips (FIGS. 12A-B). These tumor specimens are well-suited for 3D modeling because their distinct anatomic features serve as reference points by which the pathologist can realign the initial model produced by the software and orient the specimen relative to the patient. For this reason, mandibular specimens routinely offered the most vivid surface-level detail in the final 3D model. Recognizable landmarks such as the angle of the mandible, condyle, and teeth facilitated a much faster alignment and editing process for the pathologist. These bony components also provided the specimens with a rigid structure that remained consistent throughout the entire scan, thus resulting in high-quality 3D images. Conversely, specimens from the tongue, cheek, and face were malleable and liable to deform when the operator changed their orientation between the first and second scans. However, when done carefully, minimal image distortion was noted, where this was easily edited using the program's post-processing features (FIGS. 13A-B).

In some instances, annotated 3D specimen maps were used intraoperatively. However, these models have several applications in the postoperative setting. They provide a permanent record of the margins resected at the time of the initial surgery. Presenting these scans at tumor boards or conferences can support a more robust dialogue of a patient's case. If a patient's treatment relies on a multidisciplinary network of physicians, the care team can readily access these scans to better understand the patient's holistic tumor histopathology and extent of previous surgical treatment.

### 6. Conclusion

Optical 3D scanning technology provides a means by which the quality of head and neck surgical pathology can be significantly improved. The process of generating 3D models of resected tumor specimens is simple, non-invasive, and easily integrated into the current frozen section workflow. The information gathered from these scans enables seamless collaboration between the surgeon and pathologist when making intraoperative decisions. These findings offer valuable insight into the characteristics of various head and neck specimens that impact their amenability to scanning. This quality improvement initiative can be readily adapted to suit other surgical oncology specialties. Ultimately, the present disclosure systems and methods (e.g., the platform) can serve as the fundamental framework for establishing a contemporary “gold-standard” of conducting frozen section analysis and communicating margin results.

### REFERENCES

1. Maxwell J H, Thompson L D, Brandwein-Gensler M S, et al. Early Oral Tongue Squamous Cell Carcinoma: Sampling of Margins From Tumor Bed and Worse Local Control. JAMA Otolaryngol Head Neck Surg. 2015; 141 (12): 1104-1110.
2. Meier J D, Oliver D A, Varvares M A. Surgical margin determination in head and neck oncology: current clinical practice. The results of an International American Head and Neck Society Member Survey. Head Neck. 2005; 27 (11): 952-958.
3. Varvares M A, Walker R J, Chiosea S. Does a specimen-based margin analysis of early tongue cancer better predict local control? Laryngoscope. 2016; 126 (11): 2426-2427.
4. Kain J J, Birkeland A C, Udayakumar N, et al. Surgical margins in oral cavity squamous cell carcinoma: Current practices and future directions. The Laryngoscope. 2020; 130 (1): 128-138.
5 Bulbul M G, Zenga J, Tarabichi O, et al. Margin Practices in Oral Cavity Cancer Resections: Survey of American Head and Neck Society Members. Laryngoscope. 2021; 131 (4): 782-787.
6. Falk M, Ynnerman A, Treanor D, Lundstrom C. Interactive Visualization of 3D Histopathology in Native Resolution. IEEE Trans Vis Comput Graph. 2018.
7. Hovens M C, Lo K, Kerger M, et al. 3D modelling of radical prostatectomy specimens: Developing a method to quantify tumor morphometry for prostate cancer risk prediction. Pathol Res Pract. 2017; 213 (12): 1523-1529.
8. Jansen I, Lucas M, Savci-Heijink C D, et al. Three-dimensional histopathological reconstruction of bladder tumours. Diagn Pathol. 2019; 14 (1): 25.
9. Opell M B, Zeng J, Bauer J J, et al. Investigating the distribution of prostate cancer using three-dimensional computer simulation. Prostate Cancer Prostatic Dis. 2002; 5 (3): 204-208.
10. Roberts N, Magee D, Song Y, et al. Toward routine use of 3D histopathology as a research tool. Am J Pathol. 2012; 180 (5): 1835-1842.
11. Gomes J P, Costa A L, Chone C T, Altemani A M, Altemani J M, Lima C S. Three-dimensional volumetric analysis of ghost cell odontogenic carcinoma using 3-D reconstruction software: a case report. Oral Surg Oral Med Oral Pathol Oral Radiol. 2017; 123 (5): e170-e175.
12. Lorenzon L, Bini F, Quatrale M, et al. 3D MRI segmentation and 3D circumferential resection margin evaluation for a standard rectal cancer assessment. G Chir. 2018; 39 (3): 152-157.
13. Marconi S, Pugliese L, Del Chiaro M, Pozzi Mucelli R, Auricchio F, Pietrabissa A. An innovative strategy for the identification and 3D reconstruction of pancreatic cancer from C T images. Updates Surg. 2016; 68 (3): 273-278.
14. Mertzanidou T, Hipwell J H, Reis S, et al. 3D volume reconstruction from serial breast specimen radiographs for mapping between histology and 3D whole specimen imaging. Med Phys. 2017; 44 (3): 935-948.
15. Rusu M, Golden T, Wang H, Gow A, Madabhushi A. Framework for 3D histologic reconstruction and fusion with in vivo MRI: Preliminary results of characterizing pulmonary inflammation in a mouse model. Med Phys. 2015; 42 (8): 4822-4832.
16. Koivuholma A, Aro K, Makitie A, et al. Three-Dimensional Presentation of Tumor Histopathology: A Model Using Tongue Squamous Cell Carcinoma. Diagnostics (Basel). 2021; 11 (1).
17. Laakman J M, Chen S J, Lake K S, et al. Frozen Section Quality Assurance. Am J Clin Pathol. 2021; 156 (3): 461-470.
18. Li M M, Puram S V, Silverman D A, Old M O, Rocco J W, Kang S Y. Margin Analysis in Head and Neck Cancer: State of the Art and Future Directions. Ann Surg Oncol. 2019; 26 (12): 4070-4080.

TABLE 4
Summary of Specimen Models Generated
3D Scans Generated

| | |
|---|---|
| Total | 55 |
| Mandible | 20 |
| Tongue (total or hemi) | 8 |
| Larynx | 9 |
| Maxilla | 5 |
| Palate | 4 |
| Oropharynx | 5 |
| Facial Skin | 2 |
| Buccal | 1 |
| Parotid | 1 |

TABLE 5
Time Required to Render and Annotate Virtual Model
Process Time

| | |
|---|---|
| Avg. time needed to scan and render image for pathologist | 14 mins. |
| Scan time | 8 mins. |
| Post-production cleaning/editing | 6 mins. |
| Avg. time needed to export, annotate, and communicate | 20 mins. |
| final margin results via teleconference | |
| Total Time Required | 34 mins. |

### CONCLUSION

Plural instances may be provided for components, operations, or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s) described herein. In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event(” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The foregoing description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed is:

1. A system, comprising, at a first electronic device:
a display;
an input device;
one or more processors;
memory; and
one or more programs, wherein the one or more programs are stored in the memory and configured to be executed by the one or more processors, the one or more programs including instructions for a method for performing margin analysis in a tumor specimen from a subject, the method comprising:
obtaining a first three-dimensional representation of the tumor specimen and a first plurality of annotations to the first three-dimensional representation of the tumor specimen;
receiving, from a second electronic device, a first three-dimensional representation of a tumor resection defect in the subject that corresponds to the tumor specimen;
displaying, on the display, a customizable user interface comprising, for each respective tissue section in a corresponding plurality of tissue sections from the tumor specimen, one or more user affordances for providing at least a corresponding margin status of the respective tissue section, wherein:
the displaying further comprises displaying the customizable user interface at the second electronic device,
responsive to a first user interaction with respect to the customizable user interface at the first electronic device, the first user interaction is displayed on the customizable user interface at the second electronic device, and
responsive to a second user interaction with respect to the customizable user interface at the second electronic device, the second user interaction is displayed on the customizable user interface at the first electronic device;
receiving, from the second electronic device, a second plurality of annotations to the first three-dimensional representation of the tumor resection defect, wherein the second plurality of annotations comprises:
for each respective tissue section in the corresponding plurality of tissue sections that satisfies a first margin status criterion based on the corresponding margin status, an indication of one or more supplemental specimens obtained from the tumor resection defect that map to the respective tissue section; and
generating a report for the subject using, at least, the first three-dimensional representation of the tumor specimen, the first plurality of annotations, the first three-dimensional representation of the tumor resection defect, the second plurality of annotations, and the customizable user interface.

2. The system of claim 1, the one or more programs further comprising instructions for:
forming a composite three-dimensional representation of the tumor specimen by modifying the first three-dimensional representation of the tumor specimen to include the first plurality of annotations, and
communicating, to the second electronic device, one or more composite three-dimensional representation instructions for displaying an image overlay on the second electronic device comprising the composite three-dimensional representation in the form of the first plurality of annotations and the first three-dimensional representation of the tumor specimen, thereby producing a second three-dimensional representation of the tumor specimen.

3. The system of claim 1 or 2, wherein the receiving the first three-dimensional representation of a tumor resection defect further comprises displaying, on the display of the first electronic device, the first three-dimensional representation of the corresponding tumor resection defect.

4. The system of any one of claims 1-3, wherein the first plurality of annotations comprises:
a first subset of annotations, each respective annotation in the first subset of annotations indicating an orientation of the first three-dimensional representation of the tumor specimen relative to the subject,
a second subset of annotations, each respective annotation in the second subset of annotations indicating a respective tissue section in the corresponding plurality of tissue sections from the tumor specimen, and
a third subset of annotations, each respective annotation in the third subset of annotations indicating the corresponding margin status for a respective tissue section in the plurality of tissue sections, wherein the corresponding margin status indicates whether the respective tissue section has a close or positive margin based upon a pathological analysis of the respective tissue section.

5. The system of any one of claims 1-4, wherein the indication of one or more supplemental specimens obtained from the tumor resection defect that map to the respective tissue section comprises, for each respective supplemental specimen in the one or more supplemental specimens, a location and a breadth of the respective supplemental specimen relative to the first three-dimensional representation of the tumor resection defect.

6. The system of any one of claims 1-5, wherein the one or more programs further include instructions for:
forming a composite three-dimensional representation of the tumor resection defect by modifying the first three-dimensional representation of the tumor resection defect to include the second plurality of annotations, and
displaying an image overlay on the first electronic device comprising the composite three-dimensional representation in the form of the second plurality of annotations and the first three-dimensional representation of the tumor resection defect, thereby producing a second three-dimensional representation of the tumor resection defect.

7. The system of any one of claims 1-6, wherein the first margin status criterion is satisfied when the respective tissue section has a close or positive margin based upon a pathological analysis of the respective tissue section.

8. The system of any one of claims 1-7, wherein the one or more user affordances of the customizable user interface comprises, for each respective tissue section in the plurality of tissue sections:
a first affordance for inputting a first annotation that indicates an identity of the tumor specimen,
a second affordance for inputting a second annotation that indicates an identity of the respective tissue section,
a third affordance for inputting a third annotation that provides the corresponding margin status for the respective tissue section,
a fourth affordance for updating a review status of the respective tissue section from a first review status to a second review status, and
a fifth affordance for appending the one or more supplemental specimens to the plurality of tissue sections.

9. A system, comprising, at a first electronic device:
a display;
an input device;
one or more processors;
memory; and
one or more programs, wherein the one or more programs are stored in the memory and configured to be executed by the one or more processors, the one or more programs including instructions for a method for localizing close or positive margins in a tumor specimen from a subject, the method comprising:
obtaining a first three-dimensional representation of the tumor specimen, wherein the three-dimensional representation comprises more than 1000 three-dimensional points that define a boundary of the tumor specimen;
receiving a first plurality of annotations to the first three-dimensional representation of the tumor specimen, the first plurality of annotations comprising:
a first subset of annotations, each respective annotation in the first subset of annotations indicating an orientation of the first three-dimensional representation of the tumor specimen relative to the subject,
a second subset of annotations, each respective annotation in the second subset of annotations indicating a respective tissue section in a corresponding plurality of tissue sections from the tumor specimen, and
a third subset of annotations, each respective annotation in the third subset of annotations providing a corresponding margin status for a respective tissue section in the plurality of tissue sections, wherein the corresponding margin status indicates whether the respective tissue section has a close or positive margin based upon a pathological analysis of the respective tissue section; and
forming a composite three-dimensional representation of the tumor specimen by modifying the first three-dimensional representation of the tumor specimen to include the first plurality of annotations.

10. The system of claim 9, wherein the tumor specimen is from a cancer selected from the group consisting of adrenal cancer, biliary tract cancer, bladder cancer, bone/bone marrow cancer, brain cancer, breast cancer, cervical cancer, colorectal cancer, cancer of the esophagus, gastric cancer, head/neck cancer, hepatobiliary cancer, kidney cancer, liver cancer, lung cancer, ovarian cancer, pancreatic cancer, pelvis cancer, pleura cancer, prostate cancer, renal cancer, skin cancer, stomach cancer, testis cancer, thymus cancer, thyroid cancer, uterine cancer, lymphoma, melanoma, multiple myeloma, and leukemia.

11. The system of claim 9 or 10, wherein the first three-dimensional representation of the tumor specimen is obtained by a procedure comprising:
receiving a first partial representation of the tumor specimen comprising at least a 330° scan of the tumor specimen at a corresponding first orientation;
receiving a second partial representation of the tumor specimen comprising at least a 330° scan of the tumor specimen at a corresponding second orientation, wherein the second partial representation at the corresponding second orientation captures a region of the tumor specimen that is obscured at the first orientation;
determining a first plurality of landmarks, wherein:
each respective landmark in the first plurality of landmarks comprises a corresponding pair of reference positions including a first respective reference position for the first partial representation and a second respective reference position for the second partial representation, wherein the first and second reference positions are positioned at a common feature visible in the first and second partial representations,
thereby identifying a first set of landmark coordinates for the first partial representation and a second set of landmark coordinates for the second partial representation; and
using the first set of landmark coordinates for the first partial representation and the second set of landmark coordinates for the second partial representation to obtain a first alignment of the first partial representation with the second partial representation.

12. The system of any one of claims 9-11, the one or more programs further comprising instructions for:
responsive to user input, turning the composite three-dimensional representation in three-dimensional space, and wherein
the receiving the first plurality of annotations to the first three-dimensional representation of the tumor specimen comprises a manual electronic annotation of the first three-dimensional representation, for each respective annotation in the first plurality of annotations, to produce the composite three-dimensional representation.

13. The system of any one of claims 9-12, the one or more programs further comprising instructions for:
responsive to a user interaction, performing an action on all or a portion of the composite three-dimensional representation selected from the group consisting of: changing a scale of the composite three-dimensional representation, panning the composite three-dimensional representation, rotating the composite three-dimensional representation, and highlighting a user selected portion of the composite three-dimensional representation.

14. The system of any one of claims 9-13, wherein the corresponding margin status for a respective tissue section in the plurality of tissue sections is determined using frozen section analysis, wherein each respective tissue section in the plurality of tissue sections is a frozen tissue section.

15. The system of any one of claims 9-14, wherein:
a first portion of the tumor specimen comprises cancerous tissue,
a second portion of the tumor specimen comprises normal tissue, and
the corresponding margin status for a respective tissue section indicates that the respective tissue section has a close margin when a distance between a leading edge of the cancerous tissue and an outer surface of the tumor specimen satisfies a first distance criterion.

16. The system of claim 15, wherein the distance between a leading edge of the cancerous tissue and an outer surface of the tumor specimen satisfies the first distance criterion when the distance is between a first threshold distance and a second threshold distance that is greater than the first threshold distance, wherein:
the first threshold distance is at least about 1 mm, at least about 2 mm, or at least about 3 mm, and
the second threshold distance is no more than about 6 mm, no more than about 5 mm, or no more than about 4 mm.

17. The system of any one of claims 9-16, wherein:
a first portion of the tumor specimen comprises cancerous tissue,
a second portion of the tumor specimen comprises normal tissue, and
the corresponding margin status for a respective tissue section indicates that the respective tissue section has a positive margin when a distance between a leading edge of the cancerous tissue and an outer surface of the tumor specimen satisfies a second distance criterion.

18. The system of claim 17, wherein the distance between a leading edge of the cancerous tissue and an outer surface of the tumor specimen satisfies the second distance criterion when the distance is less than a third threshold distance, wherein the third threshold distance is no more than about 3 mm, no more than about 2 mm, or no more than about 1 mm.

19. The system of any one of claims 9-18, wherein the corresponding margin status further indicates whether the respective tissue section has a negative margin based upon the pathological analysis of the respective tissue section.

20. The system of claim 9-19, wherein:
a first portion of the tumor specimen comprises cancerous tissue,
a second portion of the tumor specimen comprises normal tissue, and
the corresponding margin status for a respective tissue section indicates that the respective tissue section has a negative margin when a distance between a leading edge of the cancerous tissue and an outer surface of the tumor specimen satisfies a third distance criterion.

21. The system of claim 20, wherein the distance between a leading edge of the cancerous tissue and an outer surface of the tumor specimen satisfies the third distance criterion when the distance is less than a fourth threshold distance, wherein the fourth threshold distance is at least about 4 mm, at least about 5 mm, or at least about 6 mm.

22. The system of any one of claims 9-21, wherein each respective annotation in the first subset of annotations is selected from the group consisting of: anterior, posterior, lateral, medial, right, left, superior, and inferior.

23. The system of any one of claims 9-22, the one or more programs further comprising instructions for communicating one or more composite three-dimensional representation instructions to a remote electronic device wherein the one or more composite three-dimensional representation instructions comprises:
(i) instructions for displaying an image overlay on the remote electronic device comprising the composite three-dimensional representation in the form of the first plurality of annotations and the first three-dimensional representation of the tumor specimen, thereby producing a second three-dimensional representation of the tumor specimen, and
(ii) instructions that, responsive to a first user interaction with respect to the composite three-dimensional representation of the tumor specimen at the first electronic device, apply the first user interaction to the second three-dimensional representation of the tumor specimen at the remote electronic device.

24. The system of claim 23, wherein the first user interaction is selected from the group consisting of changing a scale of the composite three-dimensional representation, panning the composite three-dimensional representation, rotating composite three-dimensional representation, and highlighting a user selected portion of the composite three-dimensional representation.

25. The system of claim 23 or 24, wherein the first user interaction comprises adding one or more annotations to the first three-dimensional representation of the tumor specimen.

26. The system of any one of claims 23-25, wherein the first electronic device and the remote electronic device are located at different facilities.

27. The system of claim 26, wherein the first electronic device is located in a frozen section (FS) laboratory, and the remote electronic device is located in an operating room (OR).

28. The system of any one of claims 23-27, wherein the first user interaction is performed using a mouse or a stylus.

29. The system of any one of claims 9-28, further comprising obtaining a first three-dimensional representation of a tumor resection defect in the subject corresponding to the tumor specimen, wherein the first three-dimensional representation of the corresponding tumor resection defect comprises more than 1000 three-dimensional points that define a boundary of the tumor resection defect, and wherein:
the obtaining the first three-dimensional representation of the corresponding tumor resection defect in the subject further comprises:
determining a completion status of the first three-dimensional representation of the corresponding tumor resection defect as complete or incomplete, and
when the completion status of the first three-dimensional representation of the corresponding tumor resection defect is incomplete:
receiving a reference representation of a defect, and
mapping the first three-dimensional representation of the corresponding tumor resection defect to the reference representation of the defect, thereby correcting one or more incomplete portions of the first three-dimensional representation of the corresponding tumor resection defect.

30. The system of claim 28 or 29, the one or more programs further comprising instructions for:
receiving a second plurality of annotations to the first three-dimensional representation of the tumor resection defect, the second plurality of annotations comprising, for each supplemental specimen in a corresponding one or more supplemental specimens, a corresponding mapping of the respective supplemental specimen to the tumor resection defect, wherein:
the one or more supplemental specimens are obtained from the subject at a location in the tumor resection defect that is adjacent to an originating location of a respective tissue section in the plurality of tissue sections that satisfies a first margin status criterion based on the corresponding margin status.

31. The system of claim 30, wherein the first margin status criterion is satisfied when the respective tissue section has a close or positive margin based upon the pathological analysis of the respective tissue section.

32. The system of claim 30 or 31, wherein the first three-dimensional representation of the corresponding tumor resection defect is received from a remote electronic device, further comprising:
displaying, after the receiving, at the first electronic device, a second three-dimensional representation of the corresponding tumor resection defect, and
applying, responsive to a first user interaction with respect to the first three-dimensional representation of the corresponding tumor resection defect at the remote electronic device, the first user interaction to the second three-dimensional representation of the corresponding tumor resection defect at the first electronic device.

33. The system of claim 32, wherein the second three-dimensional representation of the corresponding tumor resection defect is a composite three-dimensional representation of the tumor resection defect comprising the first three-dimensional representation of the tumor resection defect modified to include the second plurality of annotations.

34. The system of any one of claims 9-33, further comprising:
displaying, on the display of the first electronic device, a customizable user interface comprising, for each respective tissue section in the corresponding plurality of tissue sections from the tumor specimen,
a first affordance for providing a first annotation that indicates an identity of the tumor specimen,
a second affordance for providing a second annotation that indicates an identity of the respective tissue section,
a third affordance for providing a third annotation that provides a corresponding margin status for the respective tissue section, and
a fourth affordance for updating a review status of the respective tissue section.

35. The system of claim 34, further comprising, upon user selection of the fourth affordance, updating the review status of the respective tissue section from a first review status to a second review status.

36. The system of claim 34 or 35, wherein the customizable user interface further comprises a fifth affordance for appending one or more supplemental specimens to the plurality of tissue sections, further comprising:
when a corresponding margin status for a respective tissue section in the plurality of tissue sections satisfies a first margin status criterion:
obtaining one or more supplemental specimens from the subject at a location in the tumor resection defect that is adjacent to an originating location of the respective tissue section,
appending, upon user selection of the fifth affordance, the one or more supplemental specimens to the plurality of tissue sections,
updating, upon user selection of the fourth affordance, the review status of the respective tissue section from a first review status to a second review status, and
for each respective supplemental specimen in the one or more supplemental specimens, indicating, upon user selection of the second affordance, (i) an identity of the respective supplemental specimen and (ii) an identity of the respective tissue section in the plurality of tissue sections.

37. The system of any one of claims 34-36, further comprising communicating one or more user interface instructions to a remote electronic device, wherein the one or more user interface instructions comprises:
(i) instructions for displaying an image overlay on the remote electronic device comprising the customizable user interface, and
(ii) instructions that, responsive to a first user interaction with respect to the customizable user interface at the first electronic device, apply the first user interaction to the customizable user interface at the remote electronic device.

38. The system of any one of claims 9-37, the one or more programs further comprising instructions for generating a report for the subject using at least the first three-dimensional representation of the tumor specimen and the first plurality of annotations.

39. The system of claim 38, wherein the report is an electronic health record (EHR).

40. A method for localizing close or positive margins in a tumor specimen from a subject, the method comprising:
at a first electronic device with a display and an input device:
obtaining a first three-dimensional representation of the tumor specimen, wherein the first three-dimensional representation comprises more than 1000 three-dimensional points that define a boundary of the tumor specimen;
receiving a first plurality of annotations to the first three-dimensional representation of the tumor specimen, the first plurality of annotations comprising:
a first subset of annotations, each respective annotation in the first subset of annotations indicating an orientation of the first three-dimensional representation of the tumor specimen relative to the subject;
a second subset of annotations, each respective annotation in the second subset of annotations indicating a respective tissue section in a corresponding plurality of tissue sections from the tumor specimen; and
a third subset of annotations, each respective annotation in the third subset of annotations providing a corresponding margin status for a respective tissue section in the plurality of tissue sections, wherein the corresponding margin status indicates whether the respective tissue section has a close or positive margin based upon a pathological analysis of the respective tissue section; and
forming a composite three-dimensional representation of the tumor specimen by modifying the first three-dimensional representation of the tumor specimen to include the first plurality of annotations.

41. A non-transitory computer-readable storage medium having stored thereon program code instructions that, when executed by a processor, cause the processor to perform a method for localizing close or positive margins in a tumor specimen from a subject, the method comprising:
obtaining a first three-dimensional representation of the tumor specimen, wherein the first three-dimensional representation comprises more than 1000 three-dimensional points that define a boundary of the tumor specimen;
receiving a first plurality of annotations to the first three-dimensional representation of the tumor specimen, the first plurality of annotations comprising:
a first subset of annotations, each respective annotation in the first subset of annotations indicating an orientation of the first three-dimensional representation of the tumor specimen relative to the subject;
a second subset of annotations, each respective annotation in the second subset of annotations indicating a respective tissue section in a corresponding plurality of tissue sections from the tumor specimen; and
a third subset of annotations, each respective annotation in the third subset of annotations providing a corresponding margin status for a respective tissue section in the plurality of tissue sections, wherein the corresponding margin status indicates whether the respective tissue section has a close or positive margin based upon a pathological analysis of the respective tissue section; and
forming a composite three-dimensional representation of the tumor specimen by modifying the first three-dimensional representation of the tumor specimen to include the first plurality of annotations.

42. A method for localizing close or positive margins in a tumor specimen from a subject, the method comprising:
at a first electronic device with a display and an input device:
receiving, from a second electronic device:
(i) a first three-dimensional representation of the tumor specimen, wherein the first three-dimensional representation comprises more than 1000 three-dimensional points that define a boundary of the tumor specimen,
(ii) a first plurality of annotations to the first three-dimensional representation of the tumor specimen, the first plurality of annotations comprising:
a first subset of annotations, each respective annotation in the first subset of annotations indicating an orientation of the first three-dimensional representation of the tumor specimen relative to the subject;
a second subset of annotations, each respective annotation in the second subset of annotations indicating a respective tissue section in a corresponding plurality of tissue sections from the tumor specimen; and
a third subset of annotations, each respective annotation in the third subset of annotations providing a corresponding margin status for a respective tissue section in the plurality of tissue sections, wherein the corresponding margin status indicates whether the respective tissue section has a close or positive margin based upon a pathological analysis of the respective tissue section, and
(iii) one or more composite three-dimensional representation instructions;
displaying, after the receiving:
(i) a composite three-dimensional representation of the tumor specimen in the form of the first plurality of annotations and the first three-dimensional representation of the tumor specimen, thereby producing a second three-dimensional representation of the tumor specimen, and
(ii) responsive to a user interaction with respect to the composite three-dimensional representation of the tumor specimen at the second electronic device, the user interaction applied to the second three-dimensional representation of the tumor specimen.

43. The method of claim 42, further comprising, at the first electronic device:
obtaining a first three-dimensional representation of a corresponding tumor resection defect,
communicating one or more tumor resection defect instructions to the second electronic device, wherein the one or more tumor resection defect instructions comprises:
(i) instructions for displaying an image overlay on the second electronic device comprising the first three-dimensional representation of the corresponding tumor resection defect, thereby producing a second three-dimensional representation of the corresponding tumor resection defect, and
(ii) instructions that, responsive to a first user interaction with respect to the three-dimensional representation of the corresponding tumor resection defect at the first electronic device, apply the first user interaction to the second three-dimensional representation of the corresponding tumor resection defect at the second electronic device.

44. The method of claim 43, further comprising, when a corresponding margin status of a respective tissue section satisfies a first margin status criterion, obtaining one or more supplemental specimens from the subject at a harvesting location in the tumor resection defect that is adjacent to an originating location of the respective tissue section in the tumor specimen, and
receiving a second plurality of annotations to the first three-dimensional representation of the tumor resection defect, the second plurality of annotations comprising, for each supplemental specimen in the one or more supplemental specimens, a corresponding mapping of the respective supplemental specimen to the tumor resection defect.

45. The method of claim 44, wherein the second three-dimensional representation of the corresponding tumor resection defect on the second electronic device is a composite three-dimensional representation of the tumor resection defect comprising the first three-dimensional representation of the tumor resection defect modified to include the second plurality of annotations.

46. The method of claim 44 or 45, further comprising:
displaying, on the display of the first electronic device, a customizable user interface comprising, for each respective tissue section in the corresponding plurality of tissue sections from the tumor specimen,
a first affordance for providing a first annotation that indicates an identity of the tumor specimen,
a second affordance for providing a second annotation that indicates an identity of the respective tissue section,
a third affordance for providing a third annotation that provides a corresponding margin status for the respective tissue section, and
a fourth affordance for updating a review status of the respective tissue section.

47. The method of claim 46, further comprising communicating one or more user interface instructions to the second electronic device, wherein the one or more user interface instructions comprises:
(i) instructions for displaying an image overlay on the remote electronic device comprising the customizable user interface, and
(ii) instructions that, responsive to a first user interaction with respect to the customizable user interface at the first electronic device, apply the first user interaction to the customizable user interface at the second electronic device.

48. An electronic device, comprising:
a display;
an input device;
one or more processors;
memory; and
one or more programs, wherein the one or more programs are stored in the memory and configured to be executed by the one or more processors, the one or more programs including instructions for a method for localizing close or positive margins in a tumor specimen from a subject, the method comprising:
receiving, from a second electronic device:
(i) a first three-dimensional representation of the tumor specimen, wherein the first three-dimensional representation comprises more than 1000 three-dimensional points that define a boundary of the tumor specimen,
(ii) a first plurality of annotations to the first three-dimensional representation of the tumor specimen, the first plurality of annotations comprising:
a first subset of annotations, each respective annotation in the first subset of annotations indicating an orientation of the first three-dimensional representation of the tumor specimen relative to the subject;
a second subset of annotations, each respective annotation in the second subset of annotations indicating a respective tissue section in a corresponding plurality of tissue sections from the tumor specimen; and
a third subset of annotations, each respective annotation in the third subset of annotations providing a corresponding margin status for a respective tissue section in the plurality of tissue sections, wherein the corresponding margin status indicates whether the respective tissue section has a close or positive margin based upon a pathological analysis of the respective tissue section, and
(iii) one or more composite three-dimensional representation instructions;
displaying, after the receiving:
(i) a composite three-dimensional representation of the tumor specimen in the form of the first plurality of annotations and the first three-dimensional representation of the tumor specimen, thereby producing a second three-dimensional representation of the tumor specimen, and
(ii) responsive to a user interaction with respect to the composite three-dimensional representation of the tumor specimen at the second electronic device, the user interaction applied to the second three-dimensional representation of the tumor specimen.

49. A non-transitory computer-readable storage medium having stored thereon program code instructions that, when executed by a processor, cause the processor to perform a method for localizing close or positive margins in a tumor specimen from a subject, the method comprising:
at a first electronic device with a display and an input device:
receiving, from a second electronic device:
(i) a first three-dimensional representation of the tumor specimen, wherein the first three-dimensional representation comprises more than 1000 three-dimensional points that define a boundary of the tumor specimen,
(ii) a first plurality of annotations to the first three-dimensional representation of the tumor specimen, the first plurality of annotations comprising:
a first subset of annotations, each respective annotation in the first subset of annotations indicating an orientation of the first three-dimensional representation of the tumor specimen relative to the subject;
a second subset of annotations, each respective annotation in the second subset of annotations indicating a respective tissue section in a corresponding plurality of tissue sections from the tumor specimen; and
a third subset of annotations, each respective annotation in the third subset of annotations providing a corresponding margin status for a respective tissue section in the plurality of tissue sections, wherein the corresponding margin status indicates whether the respective tissue section has a close or positive margin based upon a pathological analysis of the respective tissue section, and
(iii) one or more composite three-dimensional representation instructions;
displaying, after the receiving:
(i) a composite three-dimensional representation of the tumor specimen in the form of the first plurality of annotations and the first three-dimensional representation of the tumor specimen, thereby producing a second three-dimensional representation of the tumor specimen, and
(ii) responsive to a user interaction with respect to the composite three-dimensional representation of the tumor specimen at the second electronic device, the user interaction applied to the second three-dimensional representation of the tumor specimen.

50. A method for localizing a tissue section in a tumor specimen from a subject to a corresponding tumor resection defect in the subject, the method comprising:
at an electronic device with a display and an input device:
obtaining a first three-dimensional representation of the tumor specimen, wherein the three-dimensional representation comprises more than 1000 three-dimensional points that define a boundary of the tumor specimen;
receiving a first plurality of annotations to the first three-dimensional representation of the tumor specimen, the first plurality of annotations comprising:
a first subset of annotations, each respective annotation in the first subset of annotations indicating an orientation of the first three-dimensional representation of the tumor specimen relative to the subject,
a second subset of annotations, each respective annotation in the second subset of annotations indicating a respective tissue section in a corresponding plurality of tissue sections from the tumor specimen, and
a third subset of annotations, each respective annotation in the third subset of annotations providing a corresponding margin status for a respective tissue section in the plurality of tissue sections, wherein the corresponding margin status indicates whether the respective tissue section has a close or positive margin based upon a pathological analysis of the respective tissue section;
obtaining a first three-dimensional representation of a corresponding tumor resection defect in the subject, wherein the first three-dimensional representation of the corresponding tumor resection defect comprises more than 1000 three-dimensional points that define a boundary of the tumor resection defect;
determining an orientation of the tumor specimen relative to the corresponding tumor resection defect; and
applying, when a corresponding margin status of a respective tissue section in the plurality of tissue sections satisfies a first margin status criterion, a second plurality of annotations to the first three-dimensional representation of the tumor resection defect, wherein:
the second plurality of annotations comprises, for each respective supplemental specimen in one or more supplemental specimens associated with the respective tissue section, a corresponding mapping of the respective supplemental specimen to the tumor resection defect.

51. The method of claim 50, wherein the one or more supplemental specimens are obtained from the subject at a harvesting location in the tumor resection defect that is adjacent to an originating location of the respective tissue section in the tumor specimen.
